(19) United States
(12) Patent Application Publication
(10) Pub. No.: US 2025/0042904 A1
(43) Pub. Date: Feb. 6, 2025

(54) COMPOUNDS USED AS SRC INHIBITORS

(71) Applicant: WIGEN BIOMEDICINE TECHNOLOGY (SHANGHAI) CO., LTD., Shanghai (CN)

(72) Inventors: Yuli XIE, Shanghai (CN); Yingming WU, Shanghai (CN); Lihui QIAN, Shanghai (CN)

(21) Appl. No.: 18/706,531

(22) PCT Filed: Nov. 15, 2022
(86) PCT No.: PCT/CN2022/131954

§ 371 Date: May 1, 2024

(30) Foreign Application Priority Data
Nov. 15, 2021 (CN) 202111350842.6

(51) Int. Cl. C07D 487/04; A61K 31/519; A61P 35/00

(52) U.S. Cl. CPC C07D 487/04; A61K 31/519; A61P 35/00

(57) ABSTRACT

Disclosed in the present invention are a class of compounds as Src inhibitors. Specifically, the present invention relates to a compound of general formula (1), a method for preparing same, and use of the compound of general formula (1) and isomers, crystalline forms, pharmaceutically acceptable salts, hydrates or solvates thereof as Src inhibitors. The compounds and the isomers, the crystalline forms, the pharmaceutically acceptable salts, the hydrates or the solvates thereof of the present invention can be used in the preparation of a medicament for treating or preventing a disease related to Src protein.

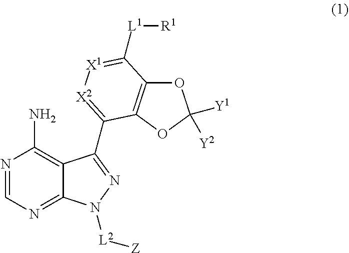

### TECHNICAL FIELD

The present invention belongs to the field of pharmaceutical chemistry, and particularly relates to a class of compounds with an inhibitory effect on Src protein, a preparation method therefor, and use of the compounds in the preparation of a medicament for treating or preventing a related disease mediated by Src.

### BACKGROUND

Src belongs to the SFK family of non-receptor tyrosine kinases (Src family kinases, SFKs), and plays an important role in multiple signaling pathways of cells. Src consists of 7 functional domains, including: 1. an N-terminal myristoylation sequence linked to an SH4 domain (Src homology 4); 2. a Src-unique domain; 3. an SH3 domain (Src homology 3); 4. an SH2 domain (Src homology 2); 5. a liner region capable of intramolecular binding to the SH3 domain; 6. a kinase domain, also known as SH1 domain (Src homology 1). The conformational changes in the Src protein will determine activated and inactivated states of the Src kinase. In normal tissues, Src is generally in an inactivated state. In tumor tissues, Src is generally overexpressed or abnormally activated.

Src is involved in multiple cell signaling processes. Src can be activated by G protein-coupled receptors (GPCRs), adhesion receptors, cytokine receptors and receptor tyrosine kinases (RTKs, such as EGFR, VEGFR, HER2 and PDGFR). As an effector of RTK signaling pathways, Src plays an important signaling role in EGFR/-ERK, PI3K/-Akt/mTOR and JAK/-STAT pathways, and promotes the development of cancer by enhancing cell proliferation, differentiation and migration.

Src has been shown to be associated with a variety of diseases, and with the intensive research on Src, overexpression or abnormal activation of Src has been found in a variety of tumors, such as breast cancer, non-small cell lung cancer, intestinal cancer, head and neck squamous cell carcinoma, pancreatic cancer, ovarian cancer and the like. There is therefore an urgent need to study and discover compounds with good activity targeting Src.

### SUMMARY

The present invention provides a compound of general formula (1) or an isomer, a crystalline form, a pharmaceutically acceptable salt, a hydrate or a solvate thereof:

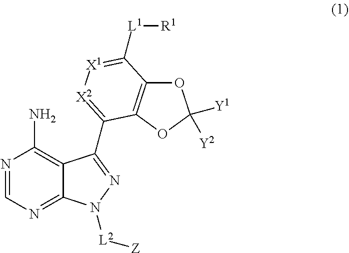

wherein in general formula (1):
X¹ is CR^{a1} or N;
X² is CR^{a2} or N;
Y¹ is H or F;
Y² is H or F;
L¹ is —C(O)—, —NR²C(O)—, —NR²C(O)O—, —OC(O)NR²—, —C(O)NR²—, —NR³C(O)NR²—, —NR²S(O)₂—, —S(O)ₚ—, —S(O)₂NR²— or —NR³S(O)₂NR²—;
R¹ is —H, —OH, —CN, (C1-C6) alkyl, (C1-C6) haloalkyl, (C2-C6) alkenyl, (C2-C6) alkynyl, (C3-C6) cycloalkyl, phenyl, (5-9 membered) heteroaryl or (4-6 membered) heterocycloalkyl, wherein the (C1-C6) alkyl, (C1-C6) haloalkyl, (C2-C6) alkenyl, (C2-C6) alkynyl, (C3-C6) cycloalkyl, phenyl, (5-9 membered) heteroaryl or (4-6 membered) heterocycloalkyl can be each independently and optionally substituted with 1, 2, 3 or 4 of the following groups: —H, halogen, —OH, —OR⁵, —CN, (C1-C6) alkyl and (C1-C6) haloalkyl;
L² is (C1-C6) alkylene, wherein the (C1-C6) alkylene can be independently and optionally substituted with 1, 2, 3 or 4 of the following groups: —H, halogen, —OH, —OR⁵, —CN, (C1-C6) alkyl, (C1-C6) haloalkyl and;

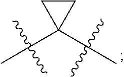 [SMILES: *C1(*)CC1]

Z is —OR⁷, —NR⁷R⁸, —NR⁶(CH₂)ₘOR⁷, —NR⁶(CH₂)ₘNR⁷R⁸, —O(CH₂)ₘOR⁶, —O(CH₂)ₘNR⁷R⁸ or (4-12 membered) heterocycloalkyl, wherein the (4-12 membered) heterocycloalkyl can be each independently and optionally substituted with 1, 2, 3 or 4 of the following groups: —H, halogen, —OH, —OR⁴, —CN, (C1-C6) alkyl, (C1-C6) haloalkyl, (C3-C6) cycloalkyl, —NR⁷R⁸, —NR⁶(CH₂)ₘOR⁷, —O(CH₂)ₘOR⁶, —O(CH₂)ₘNR⁷R⁸, —NR⁶(CH₂)ₘNR⁷R⁸, —(CH₂)ₘOR⁷, —(CH₂)ₘNR⁷R⁸ and R⁹;
R^{a1} and R^{a2} are each independently —H, —OR⁵, —CN or halogen;
R² and R³ are each independently —H, (C1-C6) alkyl, (C1-C6) haloalkyl, (C2-C6) alkenyl, (C2-C6) alkynyl or (C3-C6) cycloalkyl;
R⁴ is —H, (C1-C6) alkyl, (C1-C6) haloalkyl, (C2-C6) alkenyl, (C2-C6) alkynyl or (C3-C6) cycloalkyl;
R⁵ is —H, (C1-C6) alkyl, (C1-C6) haloalkyl or (C3-C6) cycloalkyl; R⁶, R⁷ and R⁸ are each independently —H, (C1-C6) alkyl, (C1-C6) haloalkyl, (C2-C6) alkenyl, (C2-C6) alkynyl, (C3-C6) cycloalkyl, (4-12 membered) heterocycloalkyl, phenyl or (5-6 membered) heteroaryl, wherein the (C1-C6) alkyl, (C1-C6) haloalkyl, (C2-C6) alkenyl, (C2-C6) alkynyl, (C3-C6) cycloalkyl, (4-12 membered) heterocycloalkyl, phenyl or (5-6 membered) heteroaryl can be each independently and optionally substituted with 1, 2, 3 or 4 of the following groups: —H, halogen, —OH, —OR¹⁰, —CN, (C1-C6) alkyl and (C1-C6) haloalkyl; or R⁷ and R⁸, together with the N atom to which they are linked, can form (4-12 membered) heterocycloalkyl, wherein the (4-12 membered) heterocycloalkyl can be optionally substituted with 1, 2, 3 or 4 of the following groups: —H, halogen, —OH, —OR¹⁰, —CN, (C1-C6) alkyl, (C1-C6) haloalkyl, (C1-C6) cycloalkyl, —NR¹⁰R¹¹, —NR¹⁰(CH₂)ₘOR¹¹, —O(CH₂)ₘOR¹⁰, —O(CH₂)ₘNR¹⁰R¹¹, —NR¹⁰(CH₂)ₘNR¹¹R¹², —(CH₂)ₘOR⁷, —(CH₂)ₘNR⁷R⁸ and R⁹;
R⁹ is —H or (4-9 membered) heterocycloalkyl, wherein the (4-9 membered) heterocycloalkyl can be independently and optionally substituted with 1, 2, 3 or 4 of the following groups: —H, —OH, —OR¹⁰, —CN, (C1-C6) alkyl, (C1-C6) haloalkyl or (C3-C6) cycloalkyl;
R¹⁰, R¹¹ and R¹² are each independently —H, (C1-C6) alkyl, (C1-C6) haloalkyl, (C3-C6) cycloalkyl or (4-6 membered) heterocycloalkyl; and p is an integer of 0, 1 or 2, and m is an integer of 1, 2 or 3.

In another preferred embodiment, in general formula (1), L¹ is —C(O)—, —NHC(O)—, —NHC(O)O—, —OC(O)NH—, —C(O)NH—, —NHC(O)NH—, —NHS(O)₂—, —S(O)₂NH—, —NHS(O)₂NH—, —N(CH₃)C(O)—, —N(CH₃)C(O)O—, —OC(O)N(CH₃)—, —C(O)N(CH₃)—, —N(CH₃)C(O)NH—, —NHC(O)N(CH₃)—, —N(CH₃)C(O)N(CH₃)—, —N(CH₃)S(O)₂—, —S(O)₂—, —S(O)—, —S—, —S(O)₂N(CH₃)—, —N(CH₃)S(O)₂NH—, —NHS(O)₂N(CH₃)— or —N(CH₃)S(O)₂N(CH₃)—; L¹ is preferably —NHC(O)—, —NHC(O)O—, —NHC(O)NH—, —NHC(O)O—, —OC(O)NH— or —C(O)NH—; L¹ is more preferably —NHC(O)—, —NHC(O)O— or —NHC(O)NH—; L¹ is more preferably —NHC(O)O—.

In another preferred embodiment, in general formula (1), R¹ is —H, —OH, —CN, (C1-C5) alkyl, (C1-C5) haloalkyl, (C2-C5) alkenyl, (C2-C5) alkynyl, (C3-C6) cycloalkyl, phenyl, (5-9 membered) heteroaryl or (4-6 membered) heterocycloalkyl, wherein the (C1-C5) alkyl, (C1-C5) haloalkyl, (C2-C5) alkenyl, (C2-C5) alkynyl, (C3-C6) cycloalkyl, phenyl, (5-9 membered) heteroaryl or (4-6 membered) heterocycloalkyl can be each independently and optionally substituted with 1, 2, 3 or 4 of the following groups: —H, —F, —Cl, —Br, —OH, —OCH₃, —CN, (C1-C3) alkyl and (C1-C3) haloalkyl.

In another preferred embodiment, in general formula (1), R¹ is: —H, —OH, —CN,

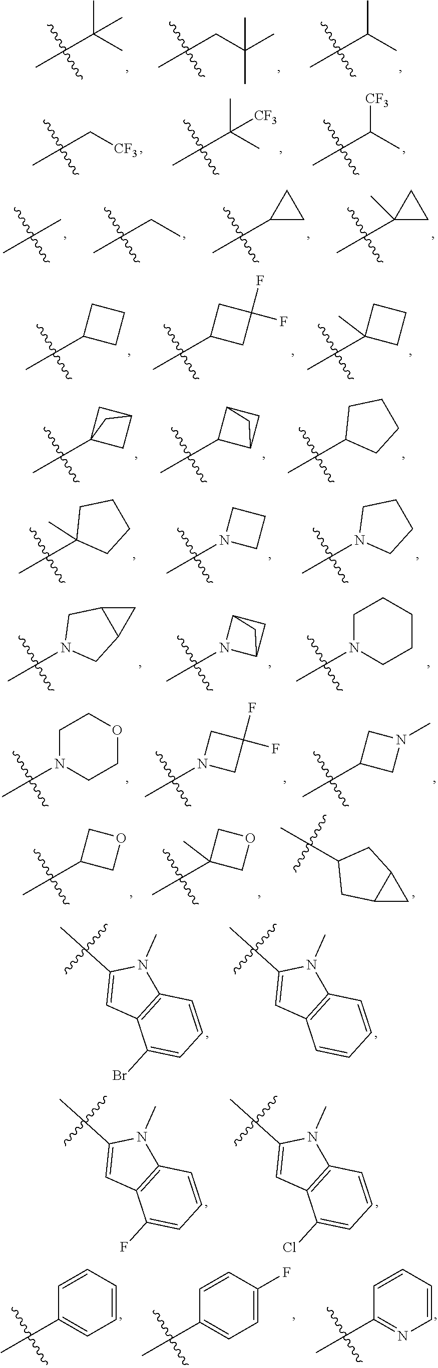

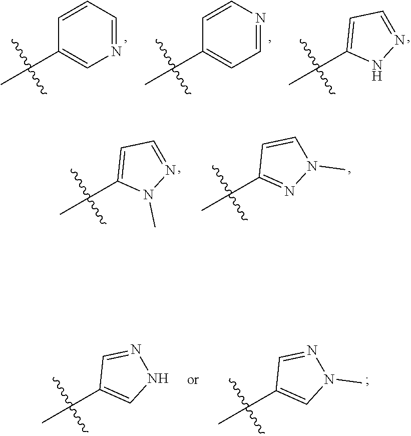

preferably 3CF₃

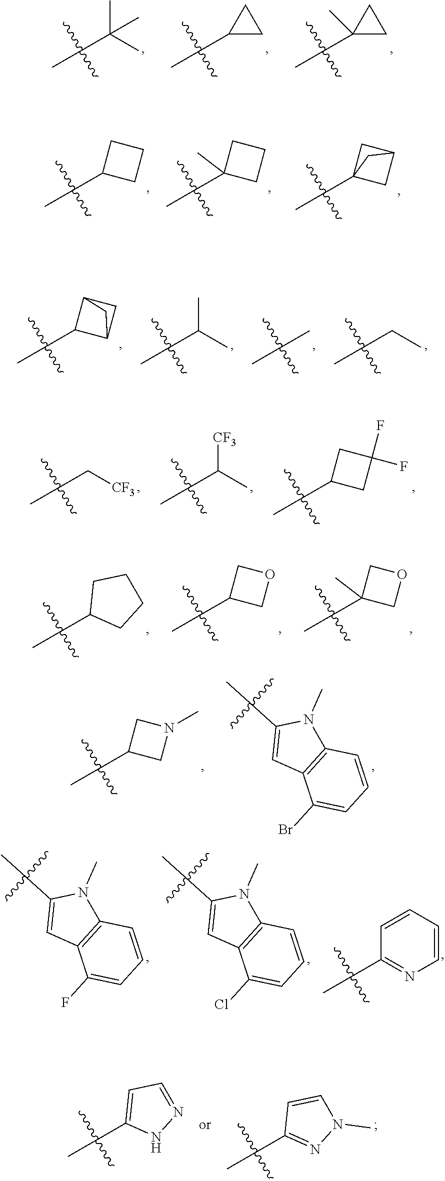

more preferably

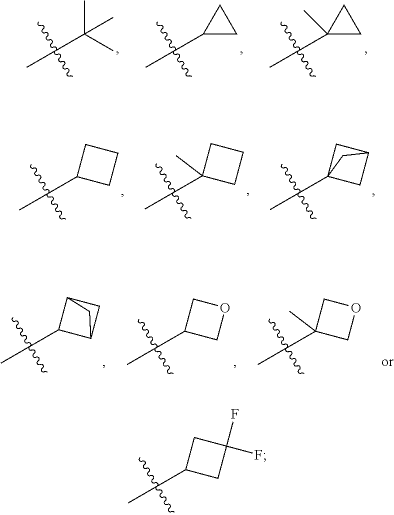

more preferably

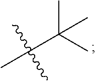 [SMILES: *C(C)(C)C]

and more preferably

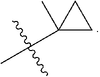 [SMILES: *C1(C)CC1]

In another preferred embodiment, in general formula (1), L² is (C1-C4) alkylene, wherein the (C1-C4) alkylene can be each independently and optionally substituted with 1, 2, 3 or 4 of the following groups: —H, —F, —OH, —OCH₃, —CN, (C1-C3) alkyl, (C1-C3) haloalkyl and

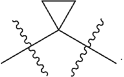

In another preferred embodiment, in general formula (1), L² is:

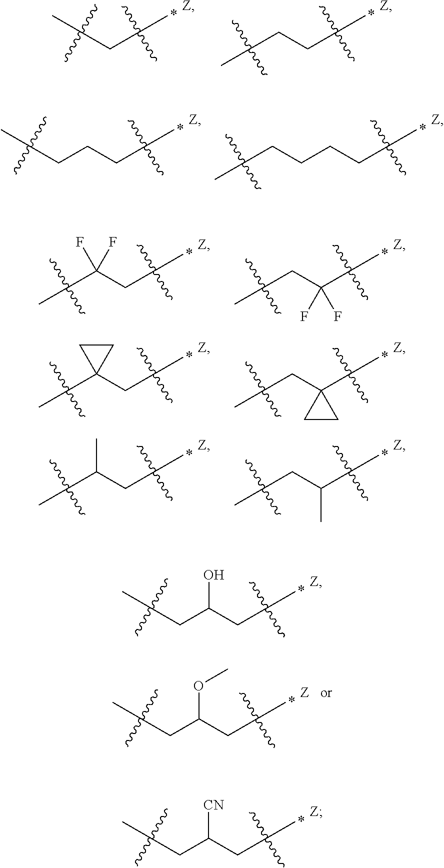

preferably

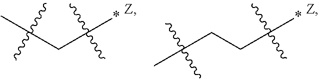

and more preferably

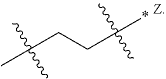

In another preferred embodiment, in general formula (1), Z is —NR⁷R⁸, —NR⁶(CH₂)₂OR⁷, —NR⁶(CH₂)₂NR⁷R⁸, —O(CH₂)₂OR⁶, —O(CH₂)₂NR⁷R⁸ or (4-11 membered) heterocycloalkyl, wherein the (4-11 membered) heterocycloalkyl can be independently and optionally substituted with 1, 2, 3 or 4 of the following groups: —H, —F, —OH, —OCH₃, —CN, (C1-C3) alkyl, (C1-C3) haloalkyl, (C3-C6) cycloalkyl, —NR⁷R⁸, —NR⁶(CH₂)₂OR⁷, —O(CH₂)₂OR⁶, —O(CH₂)₂NR⁷R⁸, —NR⁶(CH₂)₂NR⁷R⁸, —(CH₂)₂OR⁷, —(CH₂)₂NR⁷R⁸, —CH₂OR⁷, —CH₂NR⁷R⁸ and R⁹.

In another preferred embodiment, in general formula (1), R⁶, R⁷ and R⁸ are each independently —H, (C1-C3) alkyl, (C1-C3) haloalkyl, (C3-C6) cycloalkyl, (4-9 membered) heterocycloalkyl, phenyl or (5-6 membered) heteroaryl, wherein the (C1-C3) alkyl, (C1-C3) haloalkyl, (C3-C6) cycloalkyl, (4-9 membered) heterocycloalkyl, phenyl or (5-6 membered) heteroaryl can be each independently and optionally substituted with 1, 2, 3 or 4 of the following groups: —H, —F, —OH, —OCH₃, —CN, (C1-C3) alkyl and (C1-C3) haloalkyl; or R⁷ and R⁸, together with the N atom to which they are linked, can form (4-11 membered) heterocycloalkyl, wherein the (4-11 membered) heterocycloalkyl can be optionally substituted with 1, 2, 3 or 4 of the following groups: —H, —F, —OH, —OCH₃, —CN, (C1-C3) alkyl, (C1-C3) haloalkyl, (C1-C3) cycloalkyl, —NR¹⁰ R¹¹, —NR¹⁰ (CH₂)₂OR¹¹, —O(CH₂)₂OR¹⁰, —O(CH₂)₂NR¹⁰R¹¹, —NR¹⁰(CH₂)₂NR¹¹R¹², —(CH₂)₂OR⁷, —(CH₂)₂NR⁷R⁸, —CH₂OR⁷, —CH₂NR⁷R⁸ and R⁹.

In another preferred embodiment, in general formula (1), Z is:

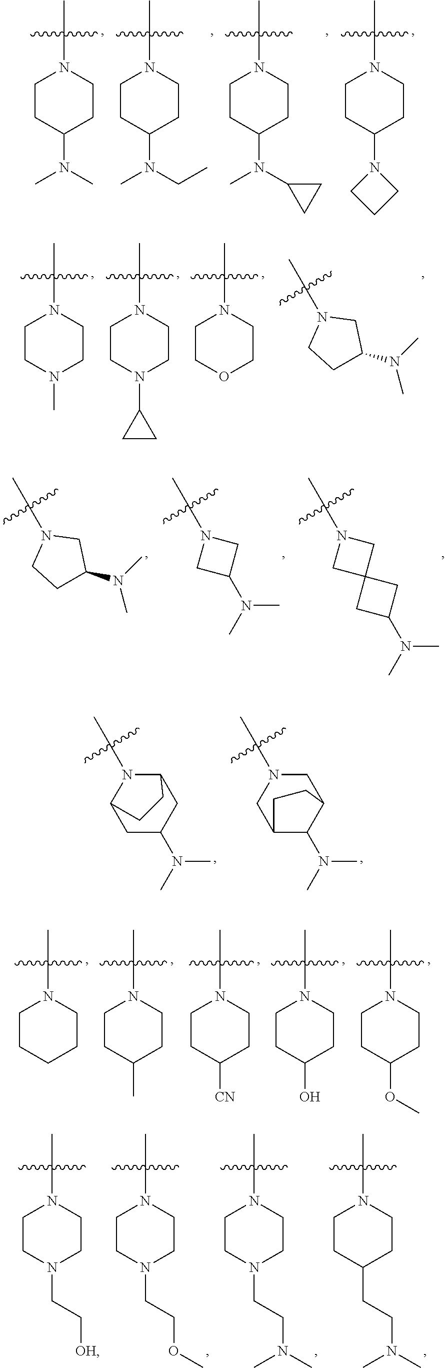

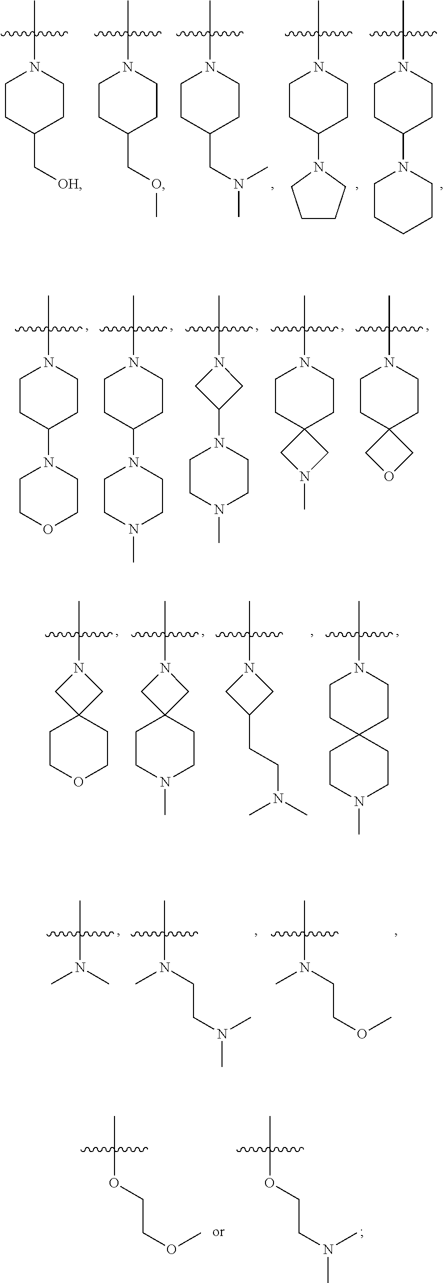

preferably

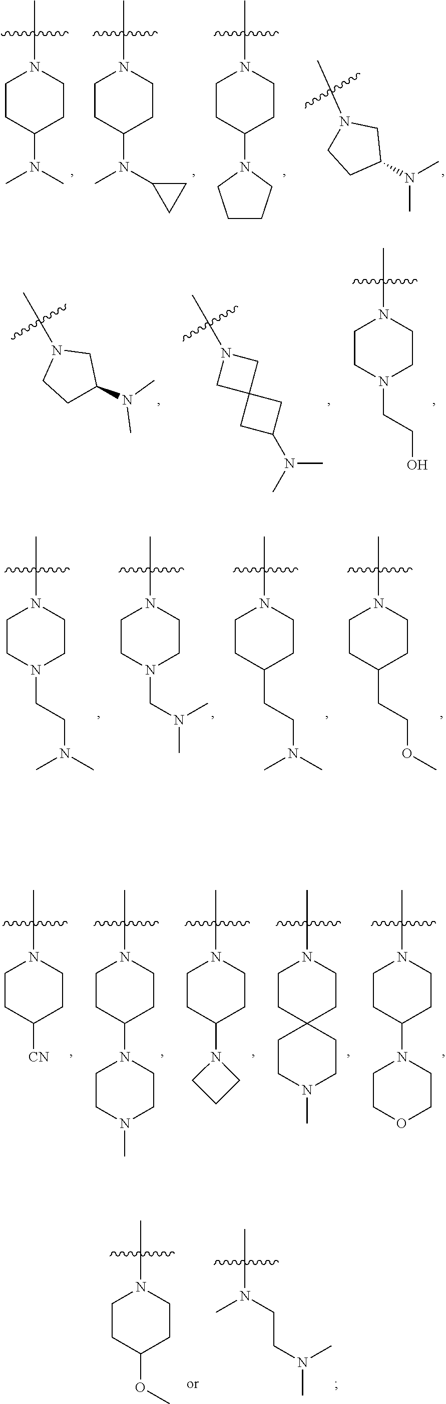

and more preferably

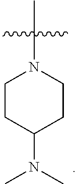 [SMILES: *N1CCC(N(C)C)CC1]

In another preferred embodiment, in general formula (1), R^{a1} and R^{a2} are each independently —H, —OCH₃, —CN or —F, preferably —H or —F, and more preferably —H.

In another preferred embodiment, in general formula (1), Y¹ and Y² are each independently —H or —F, preferably —H.

In another specific embodiment of the present invention, the compound of general formula (1) has one of the following structures:

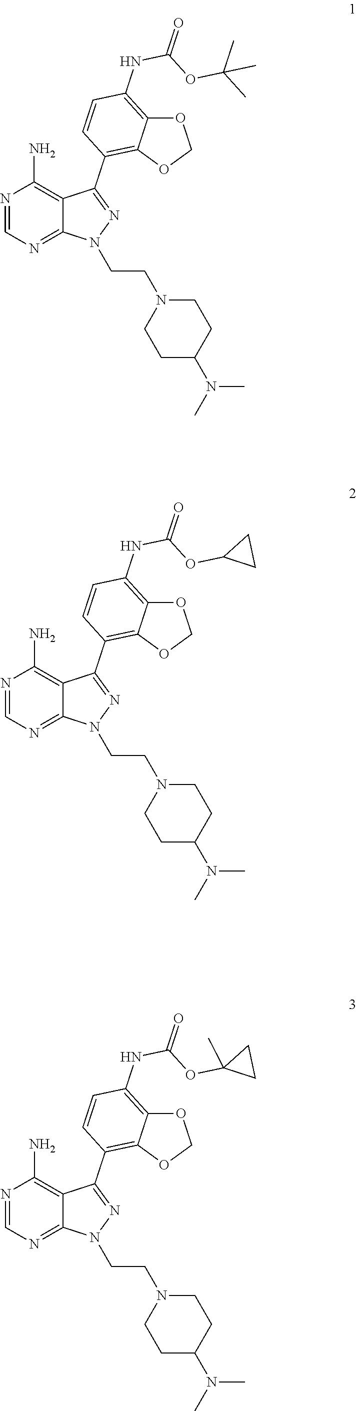

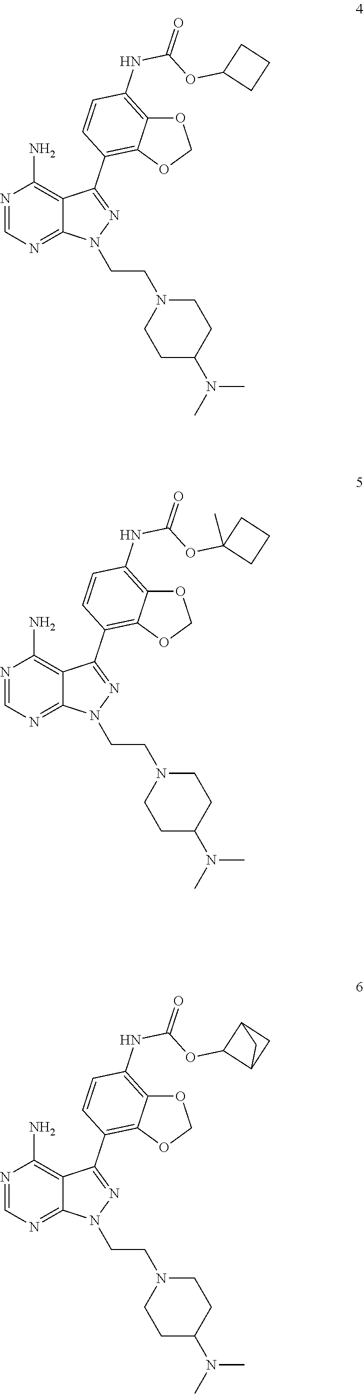

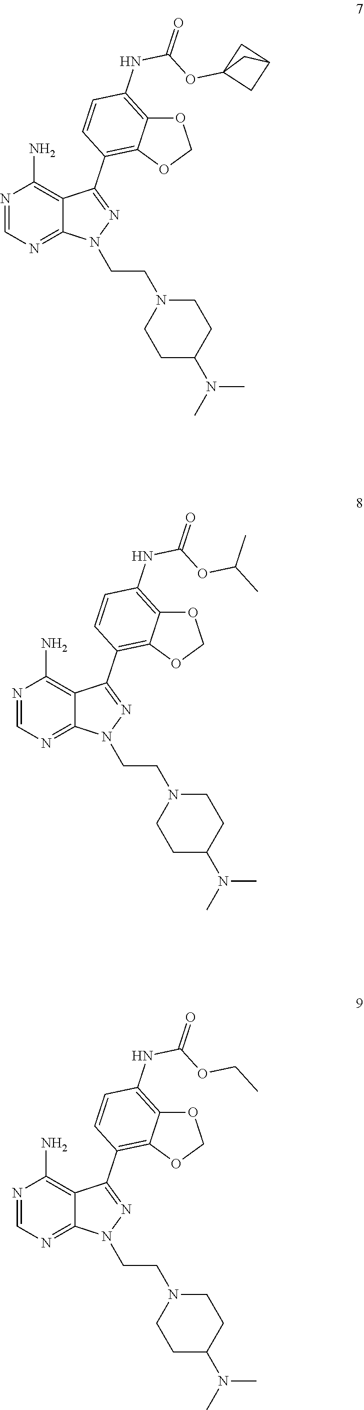

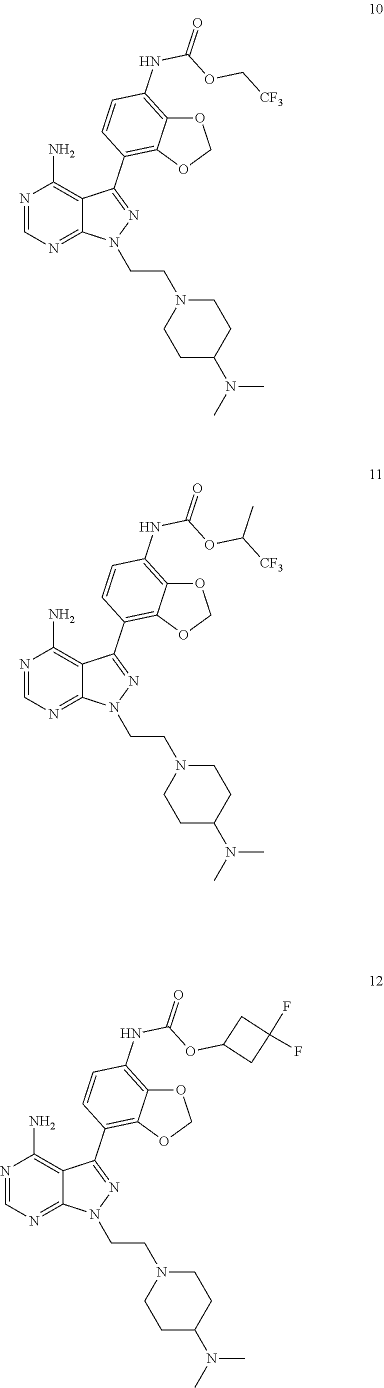

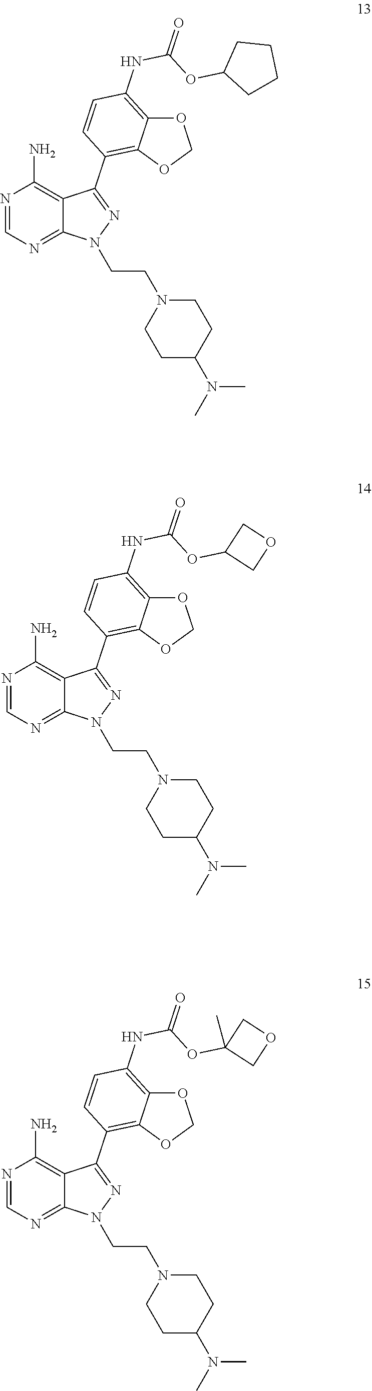

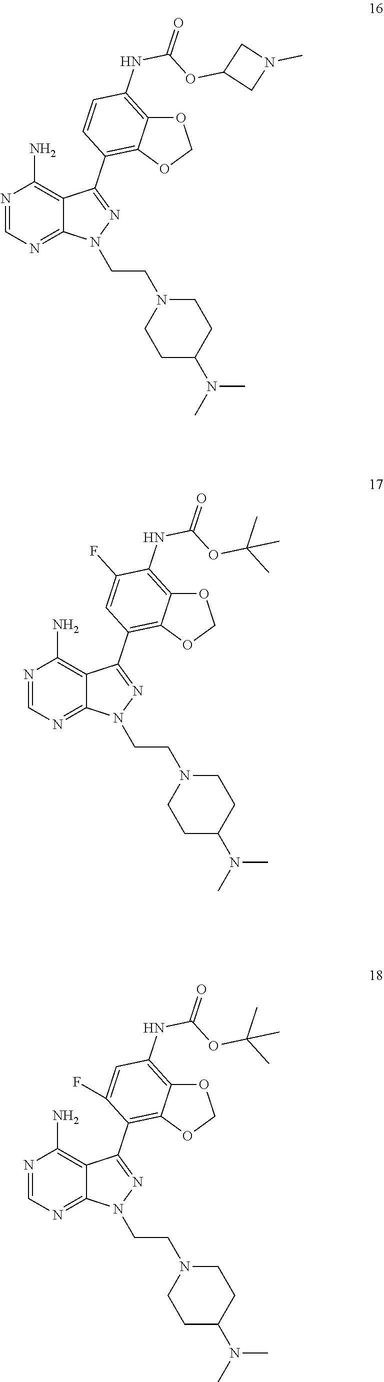

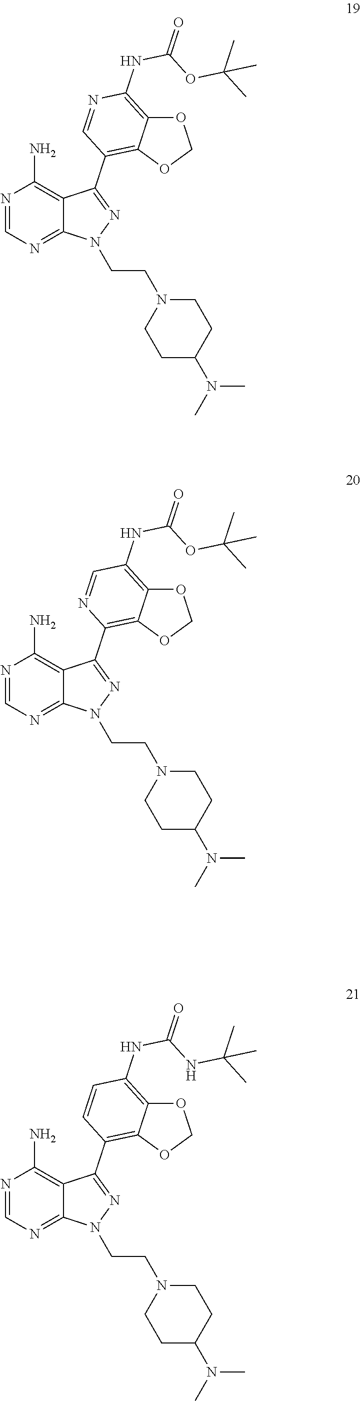

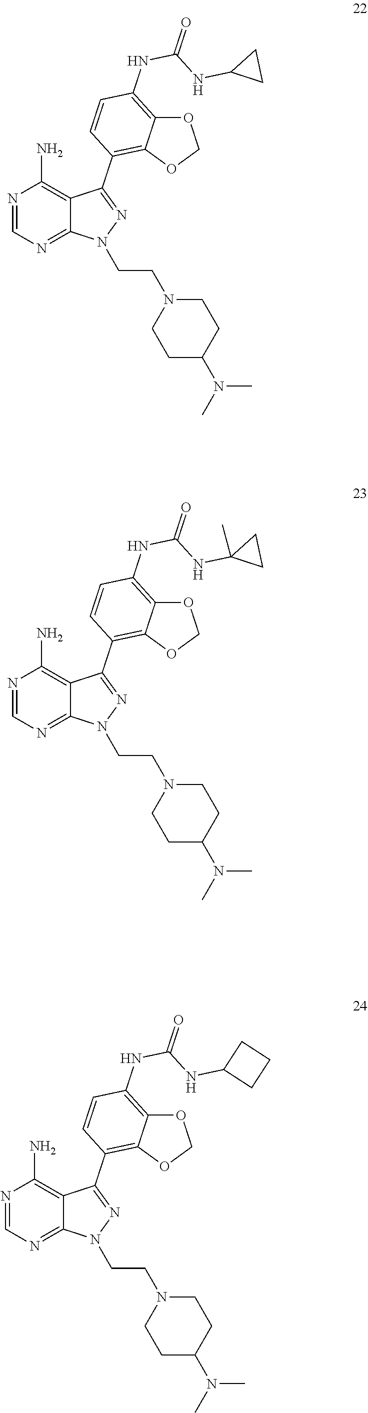

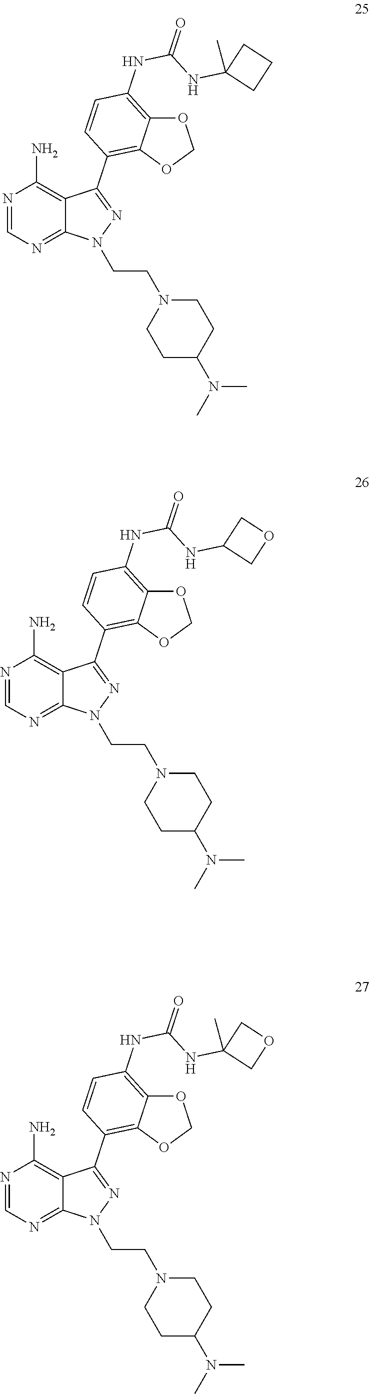

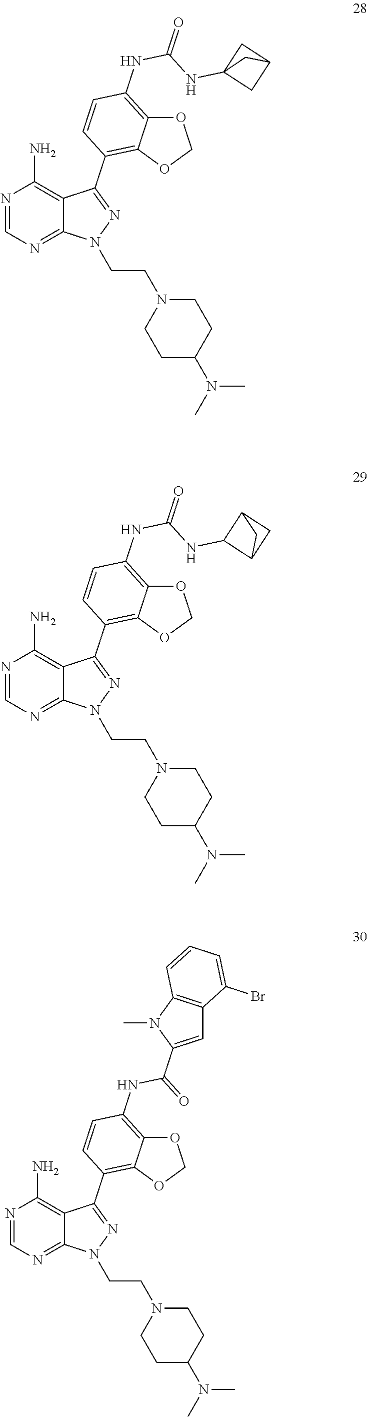

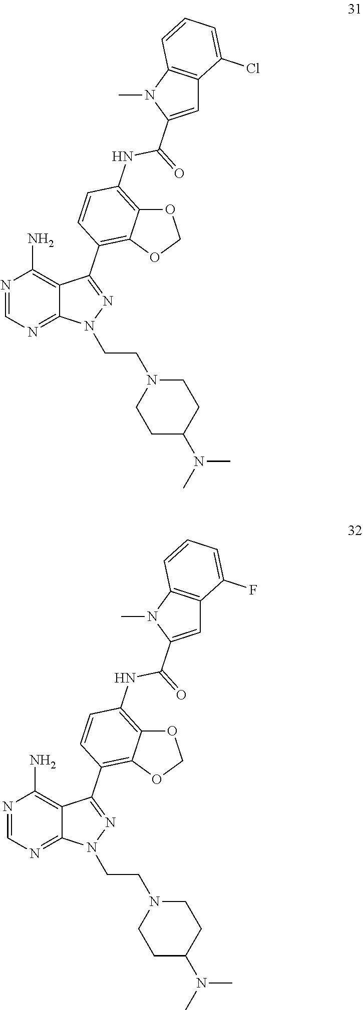

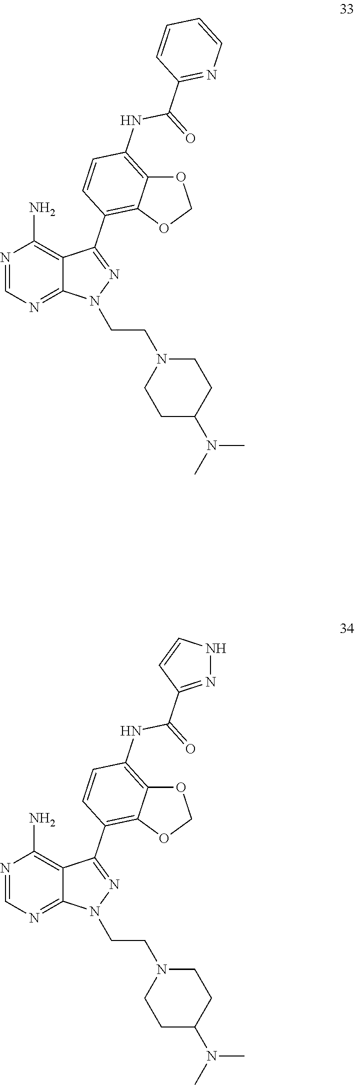

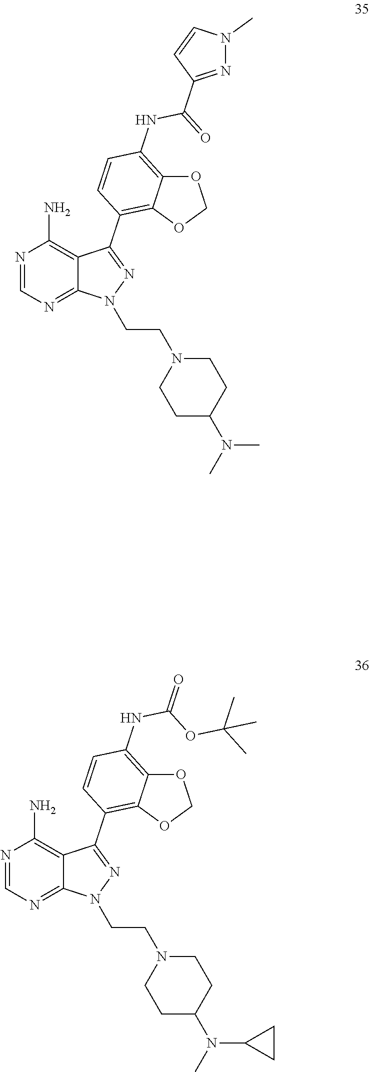

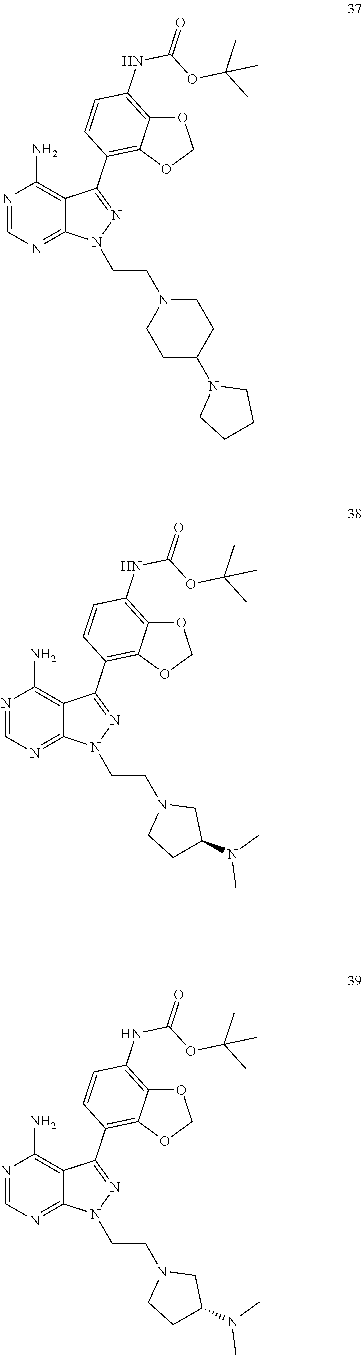

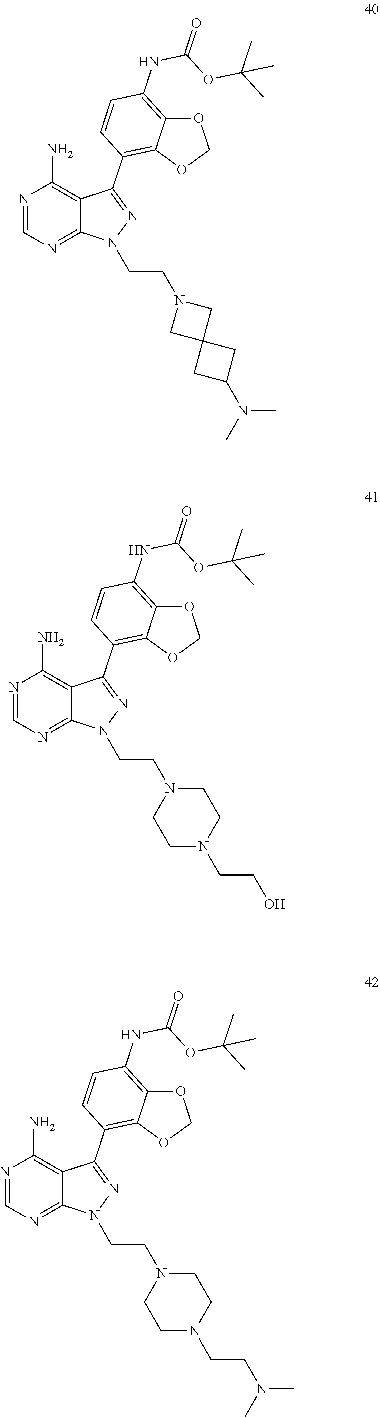

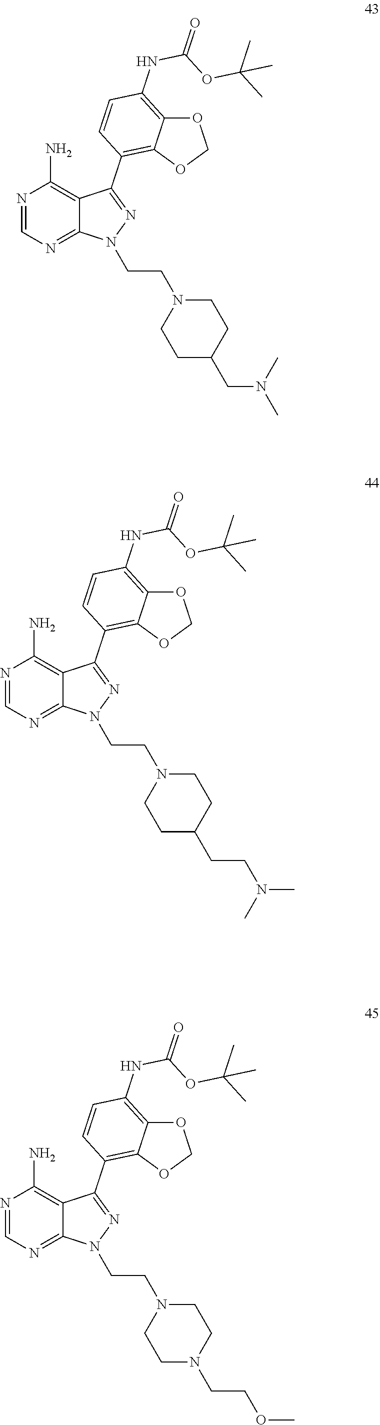

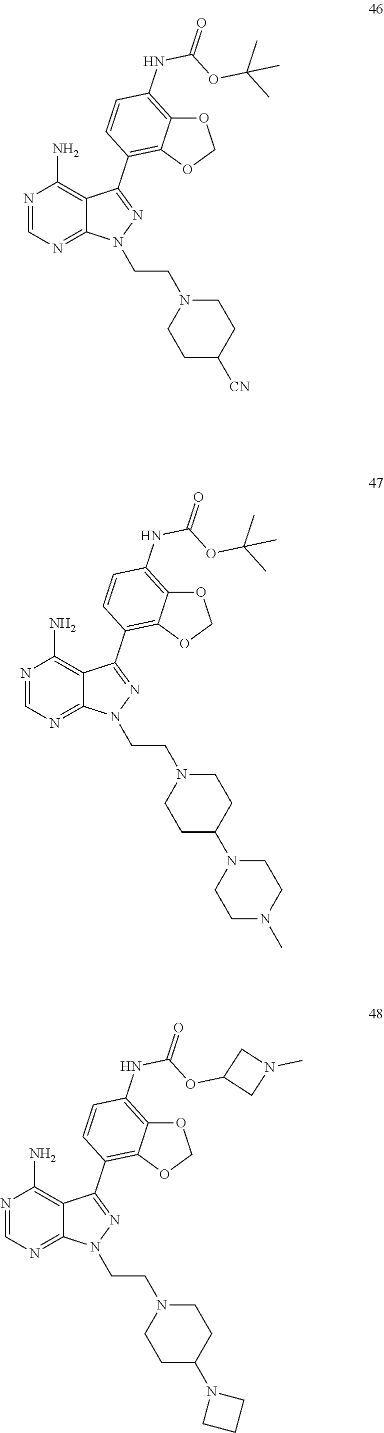

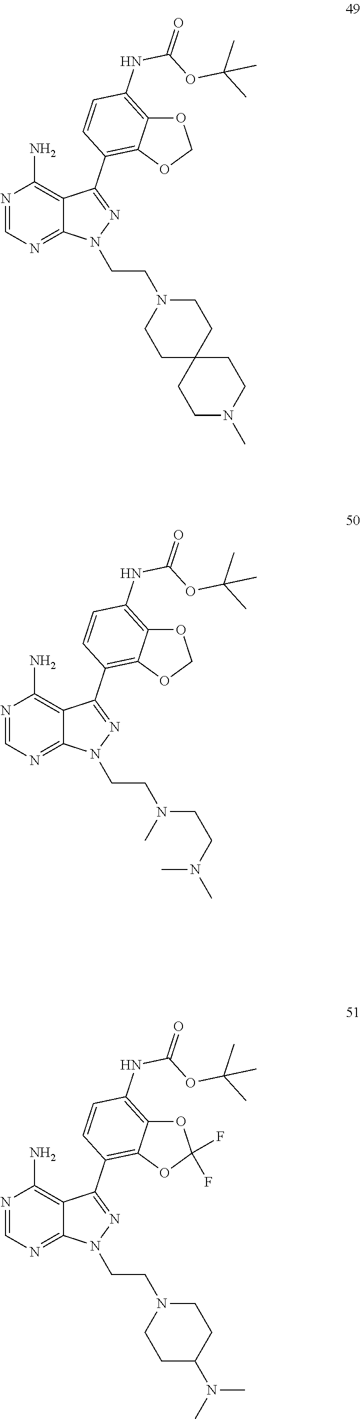

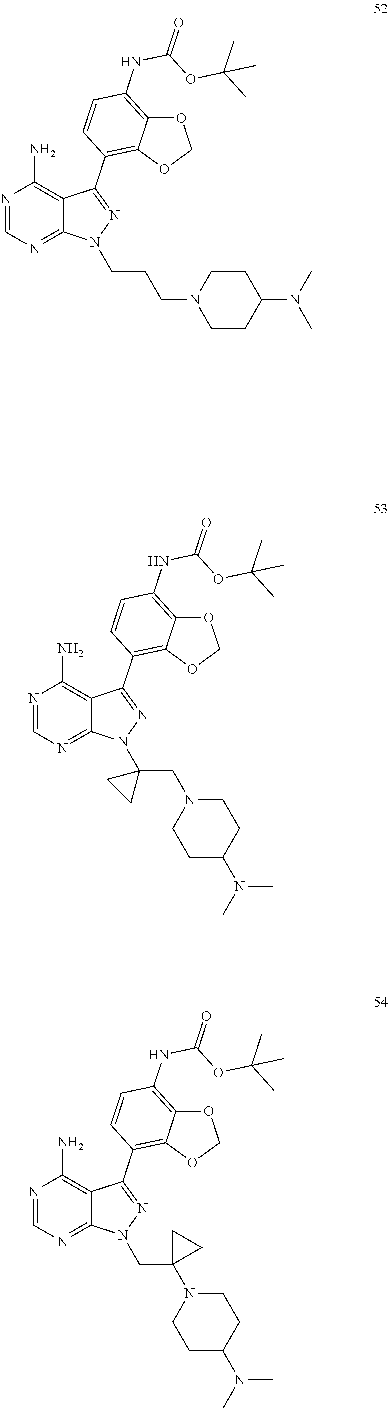

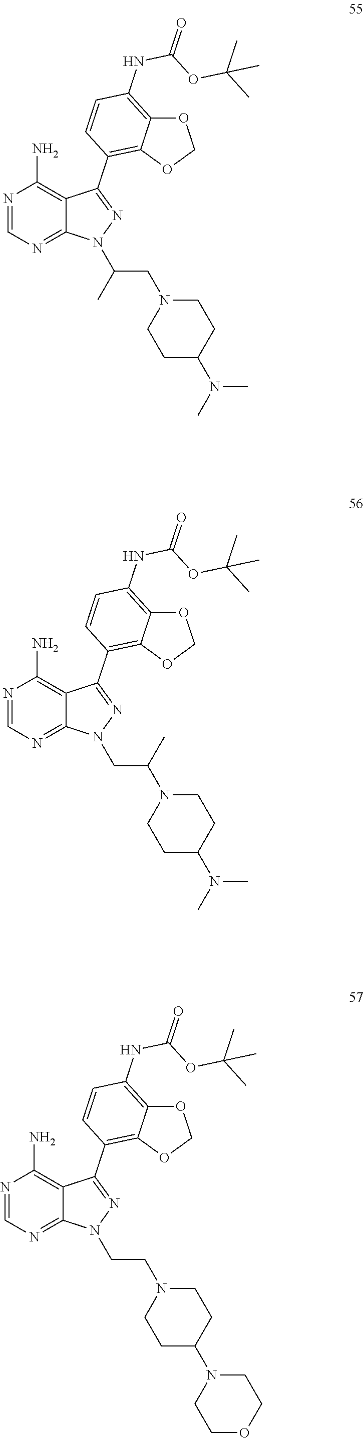

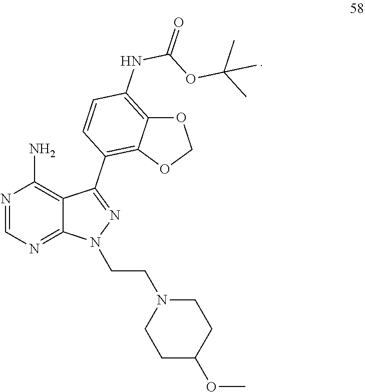

The present invention further provides a compound of general formula (2) or an isomer, a crystalline form, a pharmaceutically acceptable salt, a hydrate or a solvate thereof:

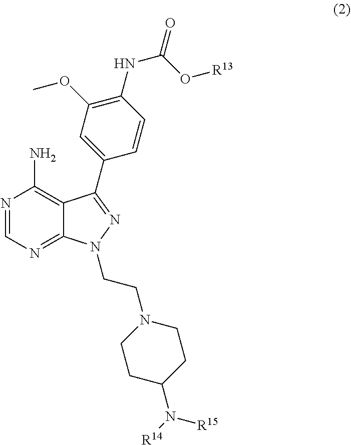

wherein in general formula (2):
R¹³ is (C1-C6) alkyl, (C3-C6) cycloalkyl or (4-6 membered) heterocycloalkyl, wherein the (C1-C6) alkyl, (C3-C6) cycloalkyl or (4-6 membered) heterocycloalkyl can be each independently and optionally substituted with 1, 2, 3 or 4 of the following groups: —H, halogen, —OH, —OR¹⁶, —CN, (C1-C6) alkyl and (C1-C6) haloalkyl;
R¹⁴ and R¹⁵ are each independently —H, (C1-C6) alkyl, (C1-C6) haloalkyl or (C3-C6) cycloalkyl, wherein the (C1-C6) alkyl, (C1-C6) haloalkyl or (C3-C6) cycloalkyl can be each independently and optionally substituted with 1, 2, 3 or 4 of the following groups: —H, halogen, —OH, —OR¹⁶, —CN, (C1-C6) alkyl and (C1-C6) haloalkyl;
when R¹³ is (C1-C6) alkyl, one of R¹⁴ and R¹⁵ must be (C3-C6) cycloalkyl;
R¹⁶ is —H, (C1-C6) alkyl, (C1-C6) haloalkyl or (C3-C6) cycloalkyl.

In another preferred embodiment, in general formula (2), R¹³ is (C1-C6) alkyl, (C3-C6) cycloalkyl or (4-6 membered) heterocycloalkyl, wherein the (C1-C6) alkyl, (C3-C6) cycloalkyl or (4-6 membered) heterocycloalkyl can be each independently and optionally substituted with 1, 2, 3 or 4 of the following groups: H, —F, —Cl, —Br, —OH, —OCH₃, —CN, (C1-C3) alkyl and (C1-C3) haloalkyl.

In another preferred embodiment, in general formula (2) R¹³ is:

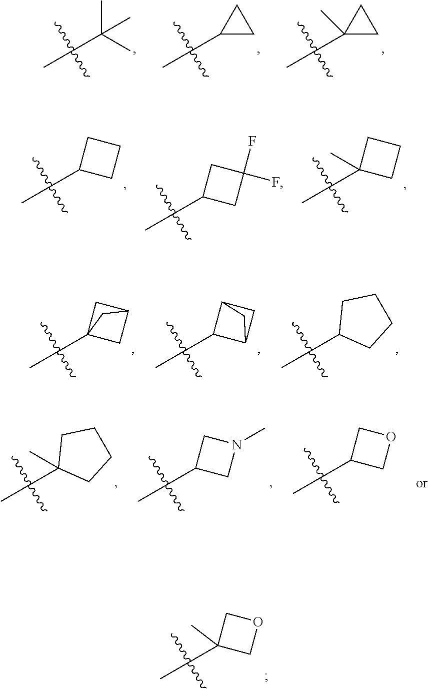

preferably

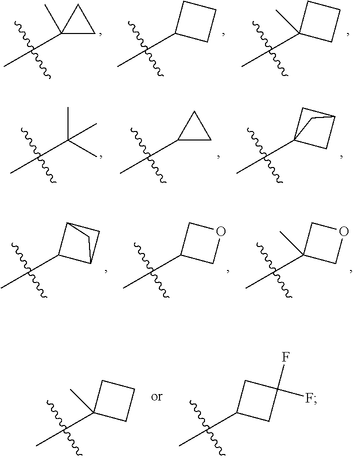

more preferably

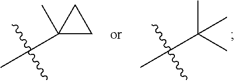

more preferably

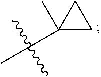 [SMILES: *C1(C)CC1]

and more preferably

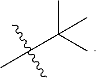 [SMILES: *C(C)(C)C]

In another preferred embodiment, in general formula (2), R¹⁴ and R¹⁵ are each independently —H, (C1-C3) alkyl, (C1-C3) haloalkyl or (C3-C6) cycloalkyl, wherein the (C1-C3) alkyl, (C1-C3) haloalkyl or (C3-C6) cycloalkyl can be each independently and optionally substituted with 1, 2, 3 or 4 of the following groups: —H, —F, —OH, —OCH₃, —CN, (C1-C3) alkyl and (C1-C3) haloalkyl; when R¹³ is (C1-C6) alkyl, one of R¹⁴ and R¹⁵ must be (C3-C6) cycloalkyl.

In another preferred embodiment, in general formula (2), structural unit

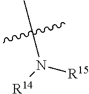

is selected
from:

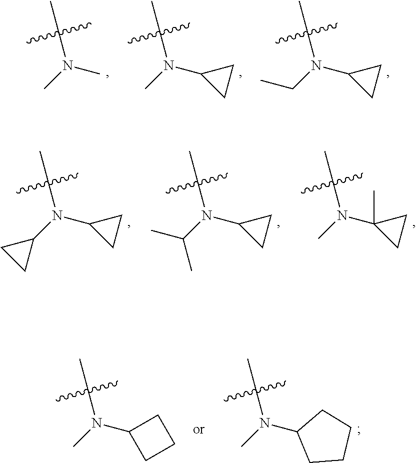

preferably

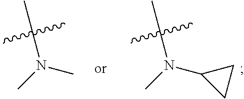

more preferably

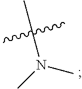

and more preferably

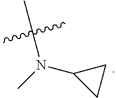 [SMILES: *N(C)C1CC1]

In another specific embodiment of the present invention, the compound of general formula (2) has one of the following structures:

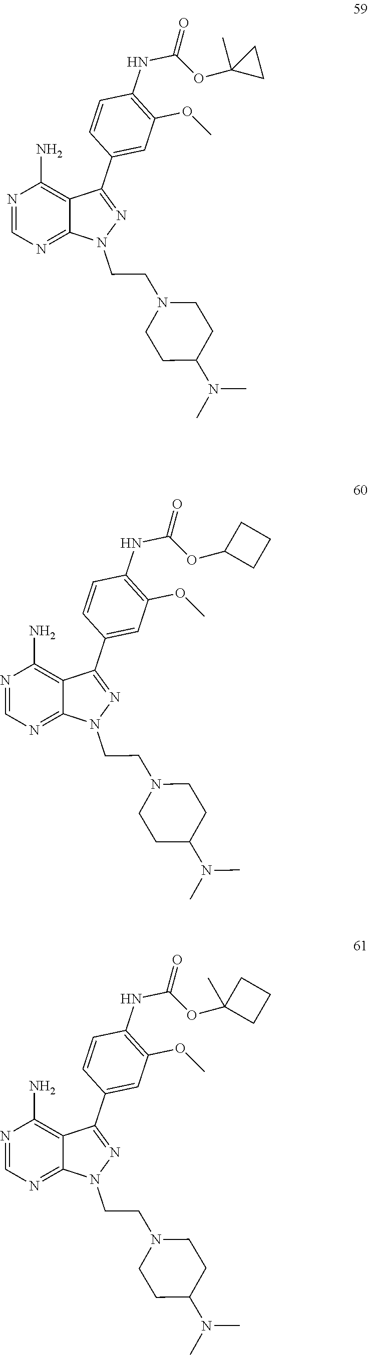

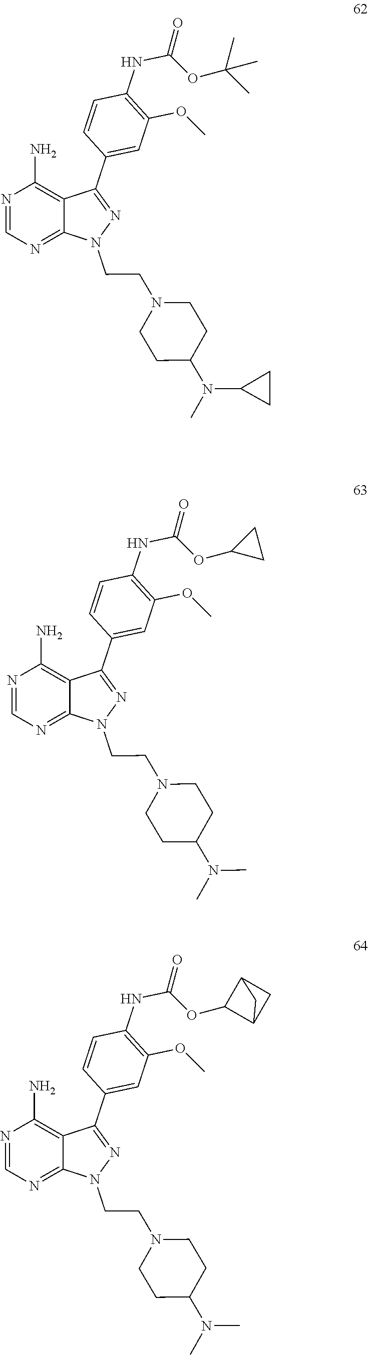

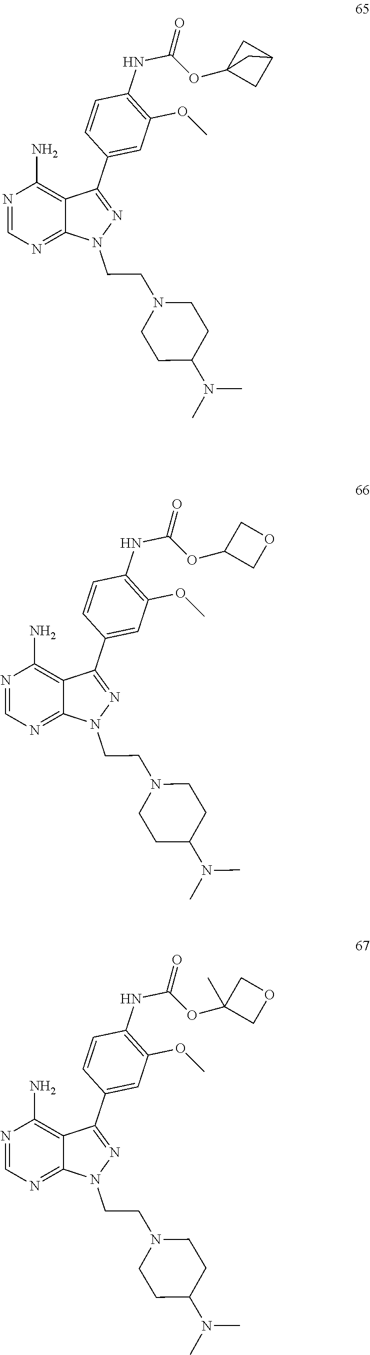

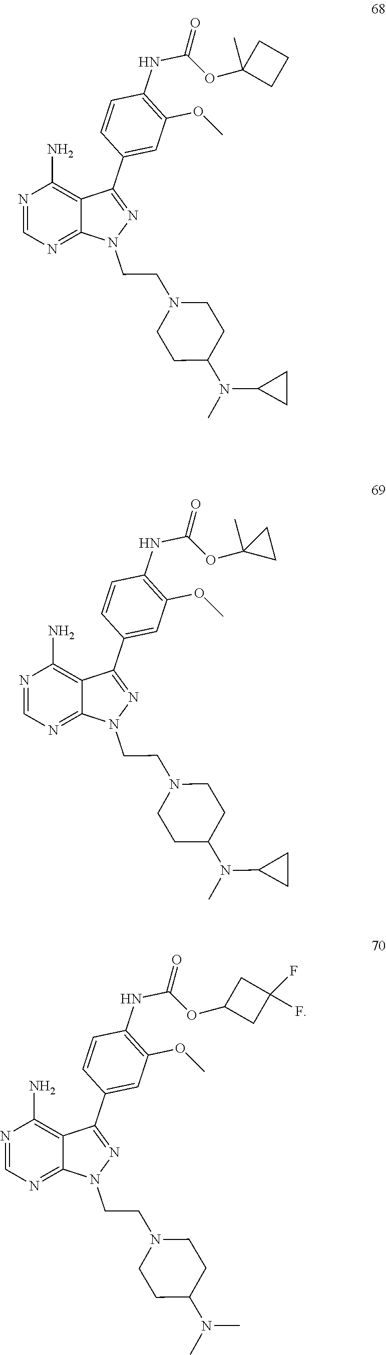

The present invention is further intended to provide a pharmaceutical composition comprising a pharmaceutically acceptable carrier, diluent and/or excipient, and the compound of general formula (1) or general formula (2) or the isomer, the crystalline form, the pharmaceutically acceptable salt, the hydrate or the solvate thereof of the present invention as an active ingredient.

The present invention is still further intended to provide use of the compound of general formula (1) or general formula (2) or the isomer, the crystalline form, the pharmaceutically acceptable salt, the hydrate or the solvate thereof of the present invention, or the pharmaceutical composition described above in the preparation of a medicament for treating, regulating or preventing a disease related to Src protein, wherein the disease is preferably cancer, and the cancer is a hematologic cancer or a solid tumor.

The present invention is even further intended to provide a method for treating, regulating or preventing a disease related to Src protein, comprising: administering to a subject a therapeutically effective amount of the compound of general formula (1) or general formula (2) or the isomer, the crystalline form, the pharmaceutically acceptable salt, the hydrate or the solvate thereof of the present invention, or the pharmaceutical composition described above.

It should be understood that both the above general description and the following detailed description of the present invention are exemplary and explanatory, and are intended to provide further explanation of the present invention claimed.

### Synthesis of Compounds

Methods for preparing the compounds of general formula (1) of the present invention are specifically described below, but these specific methods do not limit the present invention in any way.

The compounds of general formula (1) described above can be synthesized using standard synthetic techniques or well-known techniques in combination with the methods described herein. In addition, the solvents, temperatures and other reaction conditions mentioned herein can vary. Starting materials for the synthesis of the compounds can be obtained synthetically or commercially. The compounds described herein and other related compounds with different substituents can be synthesized using well-known techniques and starting materials, including the methods found in March, ADVANCED ORGANIC CHEMISTRY, 4^{th} Ed., (Wiley 1992); Carey and Sundberg, ADVANCED ORGANIC CHEMISTRY, 4^{th} Ed., Vols. A and B (Plenum 2000, 2001); and Green and Wuts, PROTECTIVE GROUPS IN ORGANIC SYNTHESIS, 3^{rd} Ed., (Wiley 1999). General methods for preparing the compounds can be changed by using appropriate reagents and conditions for introducing different groups into the molecular formulas provided herein.

In one aspect, the compounds described herein are prepared according to methods well known in the art. However, the conditions of the methods, such as reactants, solvents, bases, the amount of the compounds used, reaction temperature and time required for the reaction are not limited to the following explanation. The compounds of the present invention can also be conveniently prepared by optionally combining various synthetic methods described herein or known in the art, and such combinations can be easily determined by those skilled in the art to which the present invention pertains. In one aspect, the present invention further provides a method for preparing the compound of general formula (1), wherein the compound of general formula (1) can be prepared using general reaction scheme 1 below, and the compound of general formula (2) can be prepared using general reaction scheme 2 below:

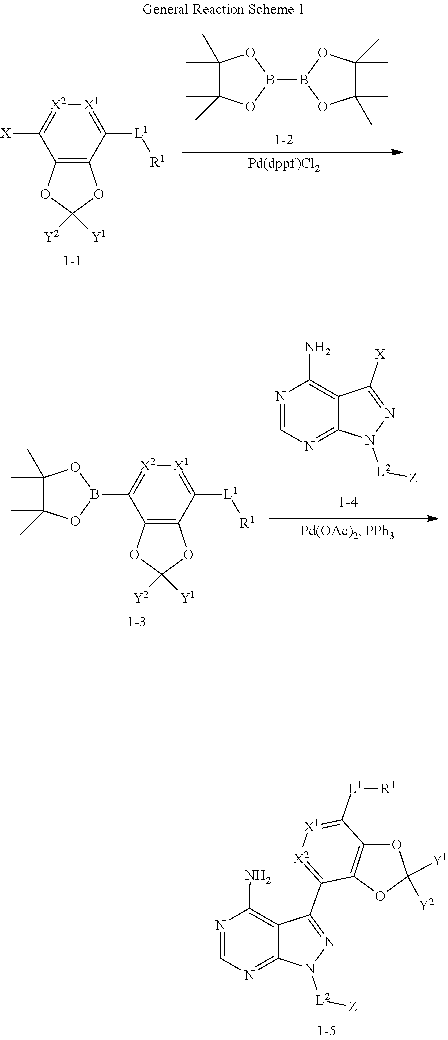

Embodiments of the compound of general formula (1) can be prepared according to general reaction scheme 1, wherein R¹, L¹, L², Z, X¹, X², Y¹ and Y² are as defined above, X represents iodine or bromine, N represents nitrogen, O represents oxygen, and B represents boron. As shown in general reaction scheme 1, compound 1-1 and compound 1-2 are subjected to a coupling reaction in the presence of Pd(dppf)Cl₂ to generate compound 1-3, and compound 1-3 and compound 1-4 are subjected to a coupling reaction in the presence of Pd(OAc)₂ and PPh₃ to generate target compound 1-5.

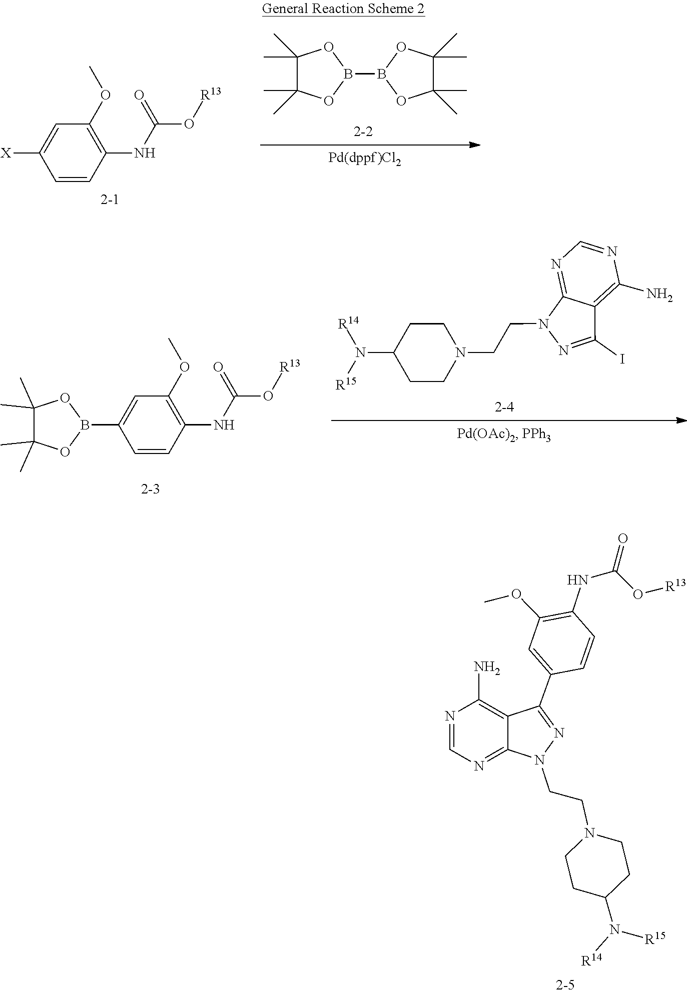

Embodiments of the compound of general formula (2) can be prepared according to general reaction scheme 2, wherein R¹³, R¹⁴ and R¹⁵ are as defined above, X represents iodine or bromine, N represents nitrogen, O represents oxygen, and B represents boron. As shown in general reaction scheme 2, compound 2-1 and compound 2-2 are subjected to a coupling reaction in the presence of Pd(dppf)Cl₂ to generate compound 2-3, and compound 2-3 and compound 2-4 are subjected to a coupling reaction in the presence of Pd(OAc)₂ and PPh₃ to generate target compound 2-5.

### Further Forms of Compounds

“Pharmaceutically acceptable” herein refers to a substance, such as a carrier or diluent, which will not lead to loss of biological activity or properties of a compound and is relatively non-toxic. For example, when an individual is given a substance, the substance will not cause undesired biological effects or interact with any component contained therein in a deleterious manner.

The term “pharmaceutically acceptable salt” refers to a form of a compound that does not cause significant irritation to the organism receiving the administration or eliminate the biological activity and properties of the compound. In certain specific aspects, the pharmaceutically acceptable salt is obtained by subjecting the compound of the general formula to a reaction with acids, e.g., inorganic acids such as hydrochloric acid, hydrobromic acid, hydrofluoric acid, sulfuric acid, phosphoric acid, nitric acid, carbonic acid and the like, organic acids such as formic acid, acetic acid, propionic acid, oxalic acid, trifluoroacetic acid, malonic acid, succinic acid, fumaric acid, maleic acid, lactic acid, malic acid, tartaric acid, citric acid, picric acid, methanesulfonic acid, benzenesulfonic acid, p-toluenesulfonic acid and the like, and acidic amino acids such as aspartic acid, glutamic acid and the like.

It should be understood that references to pharmaceutically acceptable salts include solvent addition forms or crystalline forms, especially solvates or polymorphs. A solvate contains either stoichiometric or non-stoichiometric amount of solvent and is selectively formed during crystallization in a pharmaceutically acceptable solvent such as water and ethanol. Hydrates are formed when the solvent is water, or alcoholates are formed when the solvent is ethanol. The solvates of the compound of the general formula are conveniently prepared or formed according to the methods described herein. For example, hydrates of the compound of the general formula are conveniently prepared by recrystallization in a mixed solvent of water/organic solvent, wherein the organic solvent used includes, but is not limited to, tetrahydrofuran, acetone, ethanol or methanol. Furthermore, the compounds described herein may be present in either a non-solvated form or a solvated form. In general, the solvated forms are considered equivalent to the non-solvated forms for purposes of the compounds and methods provided herein.

In other specific embodiments, the compound of the general formula is prepared in different forms including, but not limited to, amorphous, pulverized and nanoparticle forms. In addition, the compound of the general formula includes crystalline forms, and may also be polymorphs. Polymorphs include different lattice arrangements of the same elements of a compound. Polymorphs generally have different X-ray diffraction spectra, infrared spectra, melting points, density, hardness, crystalline forms, optical and electrical properties, stability and solubility. Different factors such as a recrystallization solvent, crystallization rate, and storage temperature may lead to a single dominant crystalline form.

In another aspect, the compound of the general formula may have a chiral center and/or axial chirality, and therefore may be present in the form of a racemate, a racemic mixture, a single enantiomer, a diastereomeric compound, a single diastereomer and a cis-trans isomer. Each chiral center or axial chirality will independently produce two optical isomers, and all possible optical isomers, diastereomeric mixtures, and pure or partially pure compounds are included within the scope of the present invention. The present invention is meant to include all such isomeric forms of these compounds.

The compound of the present invention may contain unnatural proportions of atomic isotopes at one or more of the atoms that constitute the compound. For example, the compound may be labeled with radioactive isotopes, such as tritium (³H), iodine-125 (¹²⁵I) and C-14 (¹⁴C). For another example, deuterium can be used to substitute a hydrogen atom to form a deuterated compound. The bond formed by deuterium and carbon is stronger than that formed by ordinary hydrogen and carbon. Compared with an undeuterated medicament, the deuterated medicament generally has the advantages of reduced toxic and side effects, increased pharmaceutical stability, enhanced efficacy, prolonged pharmaceutical in vivo half-life and the like. All isotopic variations of the compound of the present invention, whether radioactive or not, are contained within the scope of the present invention.

### Terminology

Unless otherwise stated, the terms used in the present application, including those in the specification and claims, are defined as follows. It must be noted that in the specification and the appended claims, the singular forms “a” and “an” include plural meanings unless clearly indicated otherwise. Unless otherwise stated, conventional methods for mass spectrometry, nuclear magnetic resonance spectroscopy, HPLC, protein chemistry, biochemistry, recombinant DNA techniques and pharmacology are used. As used herein, “or” or “and” refers to “and/or” unless otherwise stated.

Unless otherwise specified, “alkyl” refers to a saturated aliphatic hydrocarbon group, including linear and branched groups containing 1 to 6 carbon atoms. Lower alkyl groups containing 1 to 4 carbon atoms, such as methyl, ethyl, propyl, 2-propyl, n-butyl, isobutyl, or tert-butyl, are preferred. As used herein, “alkyl” includes unsubstituted and substituted alkyl, particularly alkyl substituted with one or more halogens. Preferred alkyl is selected from CH₃, CH₃CH₂, CF₃, CHF₂, CF₃CH₂, CF₃(CH₃)CH, ⁱPr, ⁿPr, ⁱBu, ⁿBu or ^{t}Bu.

Unless otherwise specified, “alkylene” refers to a divalent alkyl as defined above. Examples of alkylene include, but are not limited to, methylene and ethylene.

Unless otherwise specified, “alkenyl” refers to an unsaturated aliphatic hydrocarbon group containing carbon-carbon double bonds, including linear or branched groups containing 1 to 14 carbon atoms. Lower alkenyl groups containing 1 to 4 carbon atoms, such as vinyl, 1-propenyl, 1-butenyl, or 2-methylpropenyl, are preferred.

Unless otherwise specified, “alkenylene” refers to a divalent alkenyl as defined above.

Unless otherwise specified, “alkynyl” refers to an unsaturated aliphatic hydrocarbon group containing carbon-carbon triple bonds, including linear and branched groups containing 1 to 14 carbon atoms. Lower alkynyl groups containing 1 to 4 carbon atoms, such as ethynyl, 1-propynyl, or 1-butynyl, are preferred.

Unless otherwise specified, “alkynylene” refers to a divalent alkynyl as defined above.

Unless otherwise specified, “cycloalkyl” refers to a non-aromatic hydrocarbon ring system (monocyclic, bicyclic or polycyclic), and partially unsaturated cycloalkyl may be referred to as “cycloalkenyl” if the carbocyclic ring contains at least one double bond, or “cycloalkynyl” if the carbocyclic ring contains at least one triple bond. Cycloalkyl may include monocyclic or polycyclic groups (e.g., having 2, 3 or 4 fused rings) and spiro rings. In some embodiments, cycloalkyl is monocyclic. In some embodiments, cycloalkyl is monocyclic or bicyclic. The ring carbon atoms of cycloalkyl may optionally be oxidized to form an oxo or sulfido group. Cycloalkyl further includes cycloalkylene. In some embodiments, cycloalkyl contains 0, 1 or 2 double bonds. In some embodiments, cycloalkyl contains 1 or 2 double bonds (partially unsaturated cycloalkyl). In some embodiments, cycloalkyl may be fused to aryl, heteroaryl, cycloalkyl and heterocycloalkyl. In some embodiments, cycloalkyl may be fused to aryl, cycloalkyl and heterocycloalkyl. In some embodiments, cycloalkyl may be fused to aryl and heterocycloalkyl. In some embodiments, cycloalkyl may be fused to aryl and cycloalkyl. Examples of cycloalkyl include cyclopropyl, cyclobutyl, cyclopentyl, cyclohexyl, cycloheptyl, cyclopentenyl, cyclohexenyl, cyclohexadienyl, cycloheptatrienyl, norcamphanyl, norpinanyl, norcarnyl, bicyclo[1.1.1]pentyl, bicyclo[2.1.1]hexyl and the like.

Unless otherwise specified, “cycloalkylene” refers to a divalent cycloalkyl as defined above.

Unless otherwise specified, “alkoxy” refers to an alkyl group that bonds to the rest of the molecule through an ether oxygen atom. Representative alkoxy groups are those having 1-6 carbon atoms, such as methoxy, ethoxy, propoxy, isopropoxy, butoxy, isobutoxy, sec-butoxy, and tert-butoxy. As used herein, “alkoxy” includes unsubstituted and substituted alkoxy, particularly alkoxy substituted with one or more halogens. Preferred alkoxy is selected from OCH₃, OCF₃, CHF₂O, CF₃CH₂O, ⁱ⁻PrO, ⁿ⁻PrO, ⁱ⁻BuO, ⁿ⁻BuO or ^{t-}BuO.

Unless otherwise specified, “aryl” refers to an aromatic hydrocarbon group, which is monocyclic or polycyclic; for example, a monocyclic aryl ring is fused to one or more carbocyclic aromatic groups. Examples of aryl include, but are not limited to, phenyl, naphthyl, and phenanthryl.

Unless otherwise specified, “aryloxy” refers to an aryl group that bonds to the rest of the molecule through an ether oxygen atom. Examples of aryloxy include, but are not limited to, phenoxy and naphthoxy.

Unless otherwise specified, “arylene” refers to a divalent aryl as defined above. Examples of arylene include, but are not limited to, phenylene, naphthylene, and phenanthrylene.

Unless otherwise specified, “heteroaryl” refers to an aromatic group containing one or more heteroatoms (O, S, or N), and the heteroaryl is monocyclic or polycyclic. For example, a monocyclic heteroaryl ring is fused to one or more carbocyclic aromatic groups or other monocyclic heterocycloalkyl groups. Examples of heteroaryl include, but are not limited to, pyridinyl, pyridazinyl, imidazolyl, pyrimidinyl, pyrazolyl, triazolyl, pyrazinyl, quinolinyl, isoquinolinyl, furanyl, thienyl, isoxazolyl, thiazolyl, oxazolyl, isothiazolyl, pyrrolyl, indolyl, benzimidazolyl, benzofuranyl, benzothiazolyl, benzothienyl, benzoxazolyl, benzopyridinyl, pyrrolopyrimidinyl, 1H-pyrrolo[3,2-b]pyridinyl, 1H-pyrrolo[2,3-c]pyridinyl, 1H-pyrrolo[3,2-c]pyridinyl, 1H-pyrrolo[2,3-b]pyridinyl,

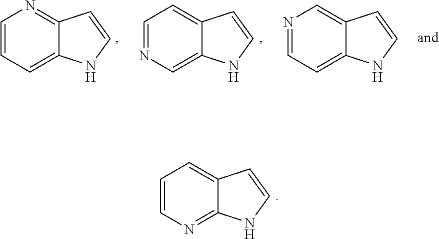

Unless otherwise specified, “heteroarylene” refers to a divalent heteroaryl as defined above.

Unless otherwise specified, “heterocycloalkyl” refers to a non-aromatic ring or ring system, which may optionally contain one or more alkenylene as part of the ring structure, having at least one heteroatom ring member independently selected from boron, phosphorus, nitrogen, sulfur, oxygen, and phosphorus. Partially unsaturated heterocycloalkyl may be referred to as “heterocycloalkenyl” if heterocycloalkyl contains at least one double bond, or “heterocycloalkynyl” if the heterocycloalkyl contains at least one triple bond. Heterocycloalkyl may include monocyclic, bicyclic, spiro ring, or polycyclic systems (e.g., having two fused or bridged rings). In some embodiments, heterocycloalkyl is a monocyclic group having 1, 2, or 3 heteroatoms independently selected from nitrogen, sulfur, and oxygen. The ring carbon atoms and heteroatoms of heterocycloalkyl may optionally be oxidized to form oxo or sulfido groups or other oxidized bonds (e.g., C(O), S(O), C(S) or S(O)2, N-oxides, etc.), or the nitrogen atoms may be quaternized. Heterocycloalkyl may be attached via a ring carbon atom or a ring heteroatom. In some embodiments, heterocycloalkyl contains 0 to 3 double bonds. In some embodiments, heterocycloalkyl contains 0 to 2 double bonds. The definition of heterocycloalkyl further includes moieties (also referred to as partially unsaturated heterocyclic rings) having one or more aromatic rings fused to (i.e., sharing a bond with) the heterocycloalkyl ring, for example, benzo-derivatives of piperidine, morpholine, azepin, thienyl, or the like. Heterocycloalkyl containing a fused aromatic ring may be attached via any ring atom, including ring atoms of the fused aromatic ring. Examples of heterocycloalkyl include, but are not limited to, azetidinyl, azepinyl, dihydrobenzofuranyl, dihydrofuranyl, dihydropyranyl, N-morpholinyl, 3-oxa-9-azaspiro[5.5]undecyl, 1-oxa-8-azaspiro[4.5]decyl, piperidinyl, piperazinyl, oxopiperazinyl, pyranyl, pyrrolidinyl, quininyl, tetrahydrofuranyl, tetrahydropyranyl, 1,2,3,4-tetrahydroquinolinyl, tropanyl, 4,5,6,7-tetrahydrothiazolo[5,4-c]pyridinyl, 4,5,6,7-tetrahydro-1H-imidazo[4,5-c]pyridine, N-methylpiperidinyl, tetrahydroimidazolyl, pyrazolidinyl, butyrolactam, valerolactam, imidazolidinonyl, hydantoinyl, dioxolanyl, phthalimidyl, pyrimidine-2,4(1H,3H)-dione, 1,4-dioxanyl, morpholinyl, thiomorpholinyl, thiomorpholinyl-S-oxide, thiomorpholinyl-S,S-oxide, piperazinyl, pyranyl, pyridonyl, 3-pyrrolinyl, thiopyranyl, pyronyl, tetrahydrothienyl, 2-azaspiro[3.3]heptanyl, indolinyl,

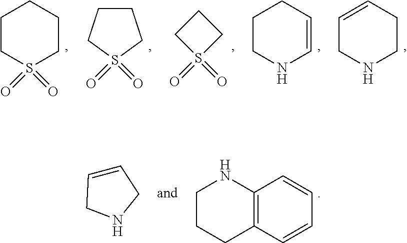

Unless otherwise specified, “heterocycloalkylene” refers to a divalent heterocycloalkyl as defined above.

Unless otherwise specified, “halogen” (or halo) refers to fluorine, chlorine, bromine or iodine.

The term “halo” (or “halogenated”) before a group name indicates that the group is partially or fully halogenated, that is, substituted in any combination with F, Cl, Br or I, preferably with F or Cl.

“Optional” or “optionally” means that the subsequently described event or circumstance may, but does not necessarily, occur, and the description includes instances where the event or circumstance occurs and instances where the event or circumstance does not occur.

The substituent “—O—CH₂—O—” means that two oxygen atoms in the substituent are linked to two adjacent carbon atoms in the heterocycloalkyl, aryl or heteroaryl, for example:

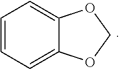

When the number of a linker group is 0, such as —(CH₂)₀—, it means that the linker group is a single bond.

When one of the variables is selected from a chemical bond, it means that the two groups linked by this variable are linked directly. For example, when L in X-L-Y represents a chemical bond, it means that the structure is actually X-Y.

The term “membered ring” includes any cyclic structure. The term “membered” is intended to refer to the number of backbone atoms that form a ring. For example, cyclohexyl, pyridinyl, pyranyl and thiopyranyl are six-membered rings, and cyclopentyl, pyrrolyl, furanyl and thienyl are five-membered rings.

The term “moiety” refers to a specific portion or functional group of a molecule. A chemical moiety is generally considered to be a chemical entity contained in or attached to a molecule. Unless otherwise stated, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ). A wavy line ( [symbol] ) represents a wedged solid bond ( [symbol] ) or a wedged dashed bond ( [symbol] ), or a wavy line ( [symbol] ) represents a straight solid bond ( [symbol] ) or a straight dashed bond ( [symbol] ). Unless otherwise stated, a single bond or a double bond is represented by [symbol] .

### Specific Pharmaceutical and Medical Terminology

The term “acceptable”, as used herein, means that a formula component or an active ingredient does not unduly and adversely affect a general therapeutic target's health.

The terms “treatment”, “treatment course”, and “therapy”, as used herein, include alleviating, inhibiting, or ameliorating a symptom or condition of a disease; inhibiting the development of complications; ameliorating or preventing underlying metabolic syndrome; inhibiting the development of a disease or symptom, e.g., controlling the progression of a disease or condition; alleviating a disease or symptom; leading to disease or symptom regression; and alleviating a complication caused by a disease or symptom, or preventing or treating a sign caused by a disease or symptom. As used herein, a compound or pharmaceutical composition, when administered, can ameliorate a disease, symptom, or condition, which particularly refers to ameliorating the severity, delaying the onset, slowing the progression, or reducing the duration of the disease. Fixed or temporary administration, or continuous or intermittent administration, may be attributed to or associated with the administration.

“Active ingredient” refers to the compound of the general formula, and pharmaceutically acceptable inorganic or organic salts of the compound of the general formula. The compound of the present invention may contain one or more asymmetric centers (chiral center or axial chirality) and thus occurs in the forms of a racemate, a racemic mixture, a single enantiomer, a diastereomeric compound and a single diastereomer. Asymmetric centers that may be present depend on the nature of the various substituents on the molecule. Each of such asymmetric centers will independently produce two optical isomers, and all possible optical isomers, diastereomeric mixtures and pure or partially pure compounds are included within the scope of the present invention. The present invention is meant to include all such isomeric forms of these compounds.

The terms such as “compound”, “composition”, “agent”, or “medicine or medicament” are used interchangeably herein and all refer to a compound or composition that, when administered to an individual (human or animal), is capable of inducing a desired pharmacological and/or physiological response by local and/or systemic action.

The term “administered, administering, or administration” refers herein to the direct administration of the compound or composition, or the administration of a prodrug, derivative, analog, or the like of the active compound.

Although the numerical ranges and parameters defining the broad scope of the present invention are approximations, the related numerical values set forth in the specific examples have been presented herein as precisely as possible. Any numerical value, however, inherently contains a standard deviation necessarily resulting from certain methods of testing. Herein, “about” generally means that the actual numerical value is within a particular numerical value or range ±10%, 5%, 1% or 0.5%. Alternatively, the term “about” indicates that the actual numerical value falls within the acceptable standard error of a mean, as considered by those skilled in the art. All ranges, quantities, numerical values, and percentages used herein (e.g., to describe an amount of a material, a length of time, a temperature, an operating condition, a quantitative ratio, and the like) are to be understood as being modified by the word “about”, except in the experimental examples or where otherwise explicitly indicated. Accordingly, unless otherwise contrarily stated, the numerical parameters set forth in the specification and the appended claims are all approximations that may vary as desired. At least, these numerical parameters should be understood as the significant digits indicated or the numerical values obtained using conventional rounding rules.

Unless otherwise defined in the specification, the scientific and technical terms used herein have the same meaning as commonly understood by those skilled in the art. Furthermore, nouns in their singular forms used in the specification encompass their plural forms, unless contradicted by context; nouns in their plural forms used also encompass their singular forms.

### Therapeutic Use

The present invention provides use of the compounds of the general formulas or the pharmaceutical compositions of the present invention in inhibiting Src protein, and therefore, in treating one or more disorders related to the activity of Src protein. Therefore, in certain embodiments, the present invention provides a method for treating Src protein-mediated disorders, which comprises the step of administering to a patient in need thereof the compounds of the general formulas or the pharmaceutically acceptable compositions thereof of the present invention.

In some embodiments, a method for treating cancer is provided, the method including administering to an individual in need thereof an effective amount of any above-mentioned pharmaceutical composition comprising the compound of a structural general formula. In some embodiments, the cancer includes, but is not limited to, hematologic malignancies (leukemias, lymphomas, and myelomas including multiple myeloma, myelodysplastic syndrome and myeloproliferative family syndrome), solid tumors (carcinomas such as prostate, breast, lung, colon, pancreatic, kidney, ovarian and soft tissue cancers, osteosarcoma, and interstitial tumors), and the like.

### Route of Administration

The compound and the pharmaceutically acceptable salt thereof of the present invention can be made into various formulations comprising a safe and effective amount of the compound or the pharmaceutically acceptable salt thereof of the present invention, and a pharmaceutically acceptable excipient or carrier, wherein the “safe and effective amount” means that the amount of the compound is sufficient to significantly improve the condition without causing serious side effects. The safe and effective amount of the compound is determined according to the age, condition, course of treatment, and other specific conditions of a treated subject.

The “pharmaceutically acceptable excipient or carrier” refers to one or more compatible solid or liquid fillers or gel substances that are suitable for human use and must be of sufficient purity and sufficiently low toxicity. “Compatible” herein means that the components of the composition are capable of intermixing with the compound of the present invention and with each other, without significantly diminishing the pharmaceutical efficacy of the compound. Examples of pharmaceutically acceptable excipients or carriers include cellulose and derivatives thereof (e.g., sodium carboxymethylcellulose, sodium ethylcellulose, or cellulose acetate), gelatin, talc, solid lubricants (e.g., stearic acid or magnesium stearate), calcium sulfate, vegetable oil (e.g., soybean oil, sesame oil, peanut oil, or olive oil), polyols (e.g., propylene glycol, glycerol, mannitol, or sorbitol), emulsifiers (e.g., Tween®), wetting agents (e.g., sodium lauryl sulfate), colorants, flavoring agents, stabilizers, antioxidants, preservatives, pyrogen-free water, etc.

When the compound of the present invention is administered, it may be administered orally, rectally, parenterally (intravenously, intramuscularly, or subcutaneously), or topically.

Solid dosage forms for oral administration include capsules, tablets, pills, pulvises, and granules. In these solid dosage forms, the active compound is mixed with at least one conventional inert excipient (or carrier), such as sodium citrate or dicalcium phosphate, or with the following ingredients: (a) fillers or extenders, such as starch, lactose, sucrose, glucose, mannitol, and silicic acid; (b) binders, such as hydroxymethyl cellulose, alginate, gelatin, polyvinylpyrrolidone, sucrose, and acacia; (c) humectants, such as glycerol; (d) disintegrants, such as agar, calcium carbonate, potato or tapioca starch, alginic acid, certain complex silicates, and sodium carbonate; (e) solution retarders, such as paraffin; (f) absorption accelerators, such as quaternary ammonium compounds; (g) wetting agents, such as cetyl alcohol and glycerol monostearate; (h) adsorbents, such as kaolin; and (i) lubricants, such as talc, calcium stearate, magnesium stearate, solid polyethylene glycol and sodium lauryl sulfate, or mixtures thereof. In the case of capsules, tablets, and pills, the dosage forms may further include buffers.

Solid dosage forms such as tablets, dragees, capsules, pills and granules can be prepared using coatings and shells such as enteric coatings and other materials well known in the art. They may include opacifying agents, and the active compound or compound in such a composition may be released in a certain part of the digestive tract in a delayed manner. Examples of embedding components that can be used are polymeric substances and wax-based substances. If necessary, the active compound can also be in microcapsule form with one or more of the excipients described above.

Liquid dosage forms for oral administration include pharmaceutically acceptable emulsions, solutions, suspensions, syrups, and elixirs. In addition to the active compound, the liquid dosage form may include inert diluents commonly used in the art, such as water or other solvents, solubilizers and emulsifiers, for example, ethanol, isopropanol, ethyl carbonate, ethyl acetate, propylene glycol, 1,3-butanediol, dimethylformamide, and oils, especially cottonseed oil, peanut oil, corn germ oil, olive oil, castor oil and sesame oil, or mixtures of these substances.

Besides such inert diluents, the composition may further include adjuvants, such as wetting agents, emulsifiers, suspending agents, sweeteners, flavoring agents, and perfuming agents.

In addition to the active compound, suspensions may include suspending agents, such as ethoxylated isostearyl alcohols, polyoxyethylene sorbitol and sorbitan esters, microcrystalline cellulose, aluminum methylate and agar, or mixtures of these substances.

Compositions for parenteral injection may include physiologically acceptable sterile aqueous or anhydrous solutions, dispersions, suspensions or emulsions, and sterile powders for redissolving into sterile injectable solutions or dispersions. Suitable aqueous and non-aqueous carriers, diluents, solvents, or excipients include water, ethanol, polyols, and suitable mixtures thereof. Dosage forms for topical administration of the compound of the present invention include ointments, pulvises, patches, sprays, and inhalants. The active ingredient is mixed under sterile conditions with a physiologically acceptable carrier and any preservatives, buffers or propellants that may be required if necessary.

The compound of the present invention may be administered alone or in combination with other pharmaceutically acceptable compounds. When the pharmaceutical composition is used, a safe and effective amount of the compound of the present invention is administered to a mammal (such as a human) to be treated, wherein the dose of administration is a pharmaceutically effective dose. For a human of 60 kg, the daily dose of administration is usually 1-2000 mg, preferably 50-1000 mg. In determining a specific dose, such factors as the route of administration, the health condition of the patient and the like will also be considered, which are well-known to skilled physicians.

The above features mentioned in the present invention or those mentioned in the examples may be combined arbitrarily. All the features disclosed in this specification may be used with any composition form and the various features disclosed in this specification may be replaced with any alternative features that provide the same, equivalent, or similar purpose. Thus, unless otherwise specified, the features disclosed herein are merely general examples of equivalent or similar features.

### DETAILED DESCRIPTION

Various specific aspects, features, and advantages of the compounds, methods, and pharmaceutical compositions described above will be set forth in detail in the following description, which will make the content of the present invention very clear. It should be understood that the detailed description and examples below describe specific examples for reference only. After reading the description of the present invention, those skilled in the art can make various changes or modifications to the present invention, and such equivalents also fall within the scope of the present application defined herein.

In all the examples, ¹H-NMR spectra were recorded with a Varian Mercury 400 nuclear magnetic resonance spectrometer, and chemical shifts are represented by 6 (ppm); silica gel for separation was 200-300 mesh silica gel if not specified, and the ratio of the eluents was a volume ratio.

The following abbreviations are used in the present invention: (Boc)₂O for di-tert-butyl dicarbonate; CDCl₃ for deuterated chloroform; Cs₂CO₃ for cesium carbonate; EtOAc for ethyl acetate; Hexane for n-hexane; HPLC for high-performance liquid chromatography; MeCN for acetonitrile; DCM for dichloromethane; DIPEA for diisopropylethylamine; Dioxane for 1,4-dioxane; DMF for N,N-dimethylformamide; DMAP for 4-(dimethylamino)pyridine; DMSO for dimethyl sulfoxide; EtOH for ethanol; h for hour; IPA for isopropanol; ISCO® for a Biotage Isolera Prime flash preparative liquid chromatograph; min for minute; K₂CO₃ for potassium carbonate; KOAc for potassium acetate; KOH for potassium hydroxide; K₃PO₄ for potassium phosphate; LiBH₄ for lithium borohydride; min for minute; MeOH for methanol; MS for mass spectrometry; NaBH(OAc)₃ for sodium triacetoxyborohydride; NaH for sodium hydrogen; NMR for nuclear magnetic resonance; NIS for iodosuccinimide; Pd/C for palladium on carbon; Pd(PPh₃)₄ for tetrakis(triphenylphosphine)palladium; Pd(OAc)₂ for palladium acetate; Pd(dppf)Cl₂ for [1,1′-bis(diphenylphosphino)ferrocene]dichloropalladium (II); PE for petroleum ether; PPh₃ for triphenylphosphine; TEA for triethylamine; TFA for trifluoroacetic acid; TsOH for p-toluenesulfonic acid; XantPhos for 4,5-bis(diphenylphosphino)-9,9-dimethylxanthene; TfOH for trifluoromethanesulfonic acid; TLC for thin-layer chromatography; XPhos for 2-dicyclohexylphosphonium-2′,4′,6′-triisopropylbiphenyl.

### Example 1. Synthesis of Compound 1

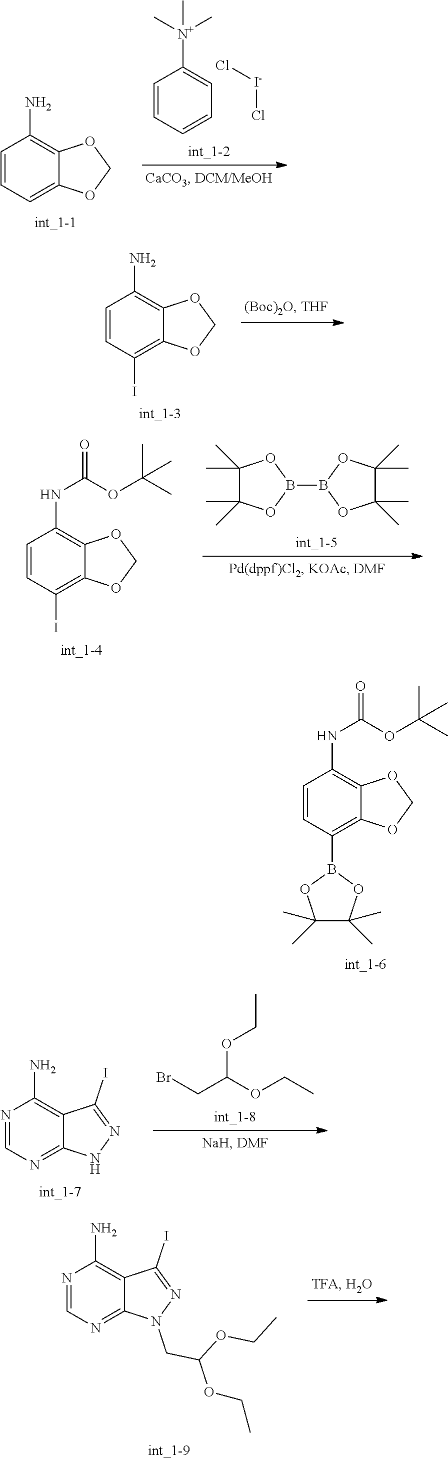

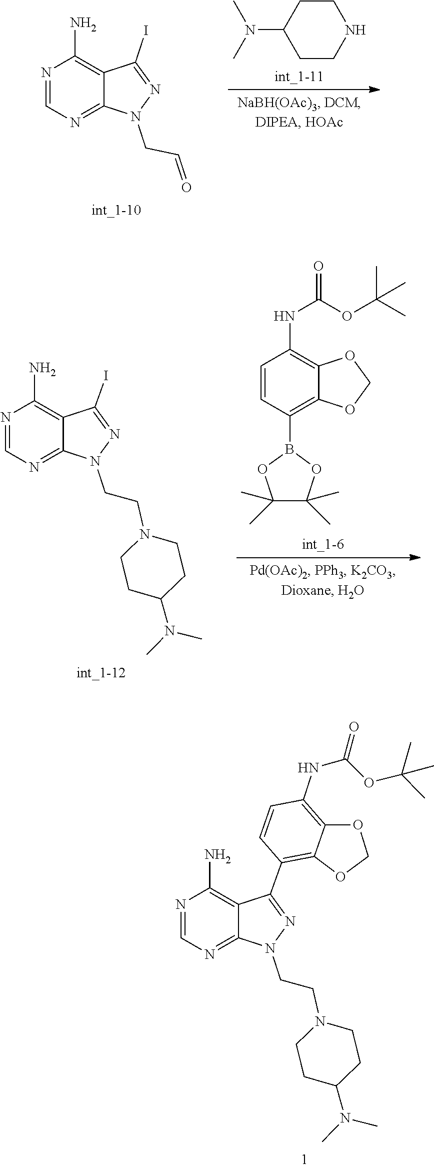

### Step 1: Synthesis of Compound Int_1-3

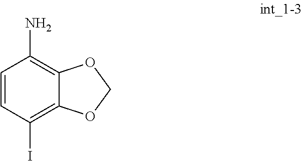 [SMILES: Nc1ccc(I)c2c1OCO2]

Int_1-2 (12.6 g, 36.1 mmol) and calcium carbonate (4.27 g, 42.7 mmol) were dissolved in a mixed solvent of methanol (70 mL) and dichloromethane (140 mL), and int_1-1 (4.50 g, 32.8 mmol) was added to the reaction solution at 30° C. After the addition was completed, the reaction solution was stirred at 30° C. for 1.5 h. LC-MS monitoring showed the reaction was completed. Water (300 mL) was added to the reaction solution. The aqueous phase was extracted with dichloromethane (300 mL×3), and the organic phase was dried over anhydrous sodium sulfate. The organic phase was concentrated under reduced pressure to give a crude product. The crude product was purified by preparative column chromatography (SiO₂, PE/EtOAc=1/0 to 4/1) to give a yellow solid (4.29 g, 49.3% yield).

¹H NMR: (400 MHz, CHLOROFORM-d) δ 6.93 (d, J=8.5 Hz, 1H), 6.17 (d, J=8.5 Hz, 1H), 5.98 (s, 2H), 3.58 (br s, 2H).

ESI-MS m/z: 264 [M+H]⁺.

### Step 2: Synthesis of Compound Int_1-4

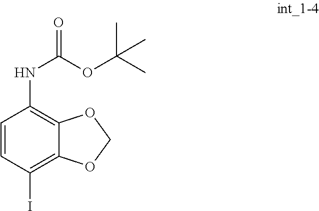 [SMILES: CC(C)(C)OC(=O)Nc1ccc(I)c2c1OCO2]

Int_1-3 (200 mg, 0.76 mmol) was dissolved in THE (10 mL), and (Boc)₂O (216 mg, 0.99 mmol) was added at room temperature. The reaction solution was reacted at 80° C. for 16 h. LC-MS monitoring showed the reaction was completed. The reaction solution was concentrated under reduced pressure to remove THE to give a crude product (260 mg), which was directly used in the next step.

ESI-MS m/z: 364 [M+H]⁺.

### Step 3: Synthesis of Compound Int_1-6

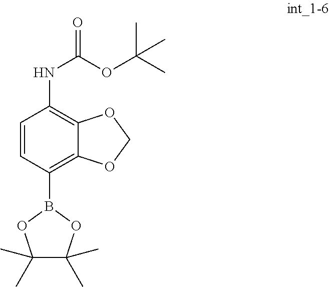 [SMILES: CC(C)(C)OC(=O)Nc1ccc(B2OC(C)(C)C(C)(C)O2)c2c1OCO2]

Int_1-4 (50 mg, 0.138 mmol) was dissolved in DMF (3 mL), and int_1-5 (42 mg, 0.165 mmol), Pd(dppf)Cl₂ (8 mg, 0.011 mmol) and potassium acetate (27 mg, 0.275 mmol) were added. The reaction system was purged with nitrogen 3 times, heated to 100° C., and stirred for 6 h under nitrogen atmosphere. LC-MS monitoring showed the reaction was completed. The reaction solution was cooled to room temperature, and water (20 mL) was slowly added to the reaction solution. The aqueous phase was extracted with ethyl acetate (20 mL×3), and the organic phase was dried over anhydrous sodium sulfate. The organic phase was concentrated under reduced pressure to give a crude product. The crude product was purified by preparative column chromatography (SiO₂, DCM/MeOH=98/2) to give a solid (10 mg, 20% yield).

ESI-MS m/z: 364, 264 [M+H]⁺.

### Step 4: Synthesis of Compound Int 1-9

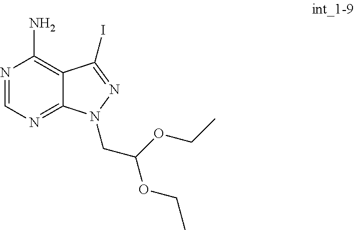 [SMILES: CCOC(Cn1nc(I)c2c(N)ncnc21)OCC]

Int_1-7 (15 g, 57.4 mmol) and int_1-8 (11.3 g, 57.4 mmol) were dissolved in DMF. NaH (2.29 g, 57.4 mmol, 60%) was added. The reaction solution was heated to 100° C. and reacted for 6 h. LC-MS monitoring showed the reaction was completed. After the reaction solution was cooled to room temperature, the reaction solution was directly concentrated under reduced pressure to remove the solvent to give a crude product (5.37 g). The crude product was purified by preparative column chromatography (SiO₂, DCM/MeOH=95/5) to give a solid (14 g, 64.6% yield).

ESI-MS m/z: 378 [M+H]⁺.

### Step 5: Synthesis of Compound Int_1-10

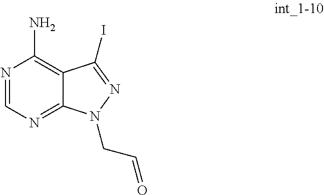 [SMILES: Nc1ncnc2c1c(I)nn2CC=O]

Int_1-9 (1 g, 2.65 mmol) was dissolved in TFA (3 mL) and water (3 mL). The reaction solution was heated to 100° C. and reacted for 1 h. LC-MS monitoring showed the reaction was completed. The reaction solution was concentrated under reduced pressure to give a crude product (1 g), which was directly used in the next step.

ESI-MS m/z: 304 [M+H]⁺.

### Step 6: Synthesis of Compound Int_1-12

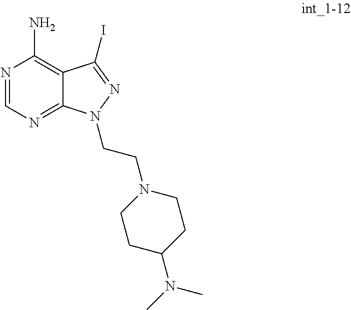 [SMILES: CN(C)C1CCN(CCn2nc(I)c3c(N)ncnc32)CC1]

Int_1-10 (500 mg, 1.64 mmol) and int_1-11 (210.2 mg, 1.64 mmol) were dissolved in dichloromethane (10 mL). Sodium triacetoxyborohydride (347.5 mg, 1.64 mmol), DIPEA (317.9 mg, 2.46 mmol) and acetic acid (150 mg, 2.46 mmol) were added to the reaction solution. The reaction solution was reacted at room temperature for 16 h. LC-MS monitoring showed the reaction was completed. Water (20 mL) was slowly added to the reaction solution. The aqueous phase was extracted with dichloromethane (20 mL×3), and the organic phase was dried over anhydrous sodium sulfate. The organic phase was concentrated under reduced pressure to give a crude product. The crude product was purified by preparative column chromatography (SiO₂, DCM/MeOH=90/10) to give a solid (580 mg, 85.1% yield).

MS (ESI): 416 [M+H]⁺.

### Step 7: Synthesis of Compound 1

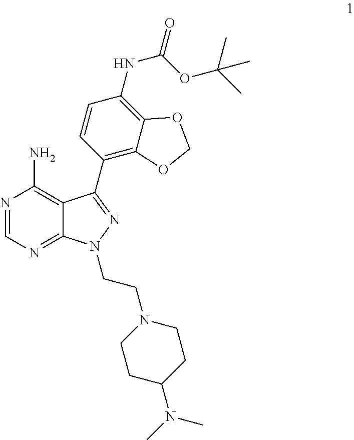 [SMILES: CN(C)C1CCN(CCn2nc(-c3ccc(NC(=O)OC(C)(C)C)c4c3OCO4)c3c(N)ncnc32)CC1]

Int_1-6 (100 mg, 0.275 mmol) and int_1-12 (171 mg, 0.413 mmol) were dissolved in Dioxane (3 mL) and H₂O (0.5 mL). Palladium acetate (4 mg, 0.014 mmol), PPh₃ (144 mg, 0.551 mmol) and potassium carbonate (76 mg, 0.551 mmol) were added to the reaction solution. The reaction solution was microwaved to 120° C. and reacted for 0.5 h under nitrogen atmosphere. LC-MS monitoring showed the reaction was completed. The reaction solution was concentrated under reduced pressure to give a crude product. The crude product was purified by preparative column chromatography (SiO₂, DCM/MeOH=90/10) and then purified by preparative HPLC to give a solid (10 mg, 7% yield).

¹H NMR (400 MHz, DMSO-d6) δ 8.98 (s, 1H), 8.19 (s, 1H), 7.13 (d, J=8.6 Hz, 1H), 6.92 (d, J=8.6 Hz, 1H), 6.07 (s, 2H), 4.39 (t, J=6.8 Hz, 2H), 2.92 (d, J=11.2 Hz, 2H), 2.73 (t, J=6.8 Hz, 2H), 2.16 (d, J=3.2 Hz, 6H), 2.12-2.01 (m, 1H), 2.01-1.87 (m, 2H), 1.66 (d, J=11.7 Hz, 2H), 1.45 (s, 6H), 1.23 (ddt, J=30.8, 11.6, 6.2 Hz, 2H).

MS (ESI): 525 [M+H]⁺.

### Examples 2-58. Synthesis of Compounds 2-58

The target compounds 2-58 in Table 1 could be obtained by the synthesis methods described above using different starting materials.

TABLE 1

| | | MS |
|---|---|---|
| Compound | Compound Structure | (M + H)⁺ |
| 2 | 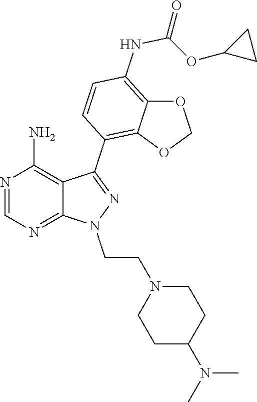 [SMILES: CN(C)C1CCN(CCn2nc(-c3ccc(NC(=O)OC4CC4)c4c3OCO4)c3c(N)ncnc32)CC1] | 509 |
| 3 | 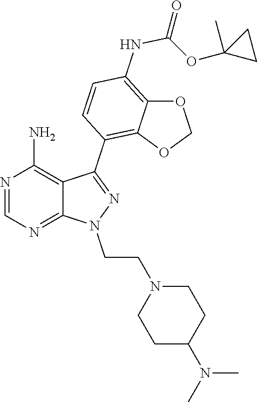 [SMILES: CN(C)C1CCN(CCn2nc(-c3ccc(NC(=O)OC4(C)CC4)c4c3OCO4)c3c(N)ncnc32)CC1] | 523 |
| 4 | 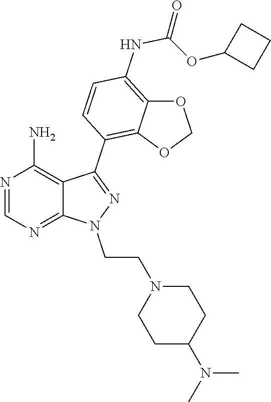 [SMILES: CN(C)C1CCN(CCn2nc(-c3ccc(NC(=O)OC4CCC4)c4c3OCO4)c3c(N)ncnc32)CC1] | 523 |
| 5 | 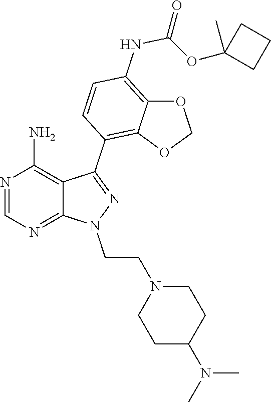 [SMILES: CN(C)C1CCN(CCn2nc(-c3ccc(NC(=O)OC4(C)CCC4)c4c3OCO4)c3c(N)ncnc32)CC1] | 537 |
| 6 | 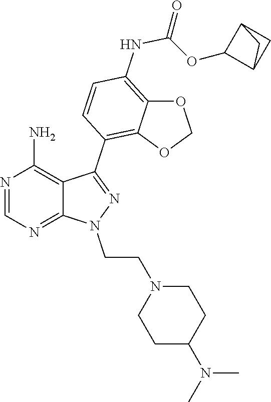 [SMILES: CN(C)C1CCN(CCn2nc(-c3ccc(NC(=O)OC4C5CC4C5)c4c3OCO4)c3c(N)ncnc32)CC1] | 535 |
| 7 | 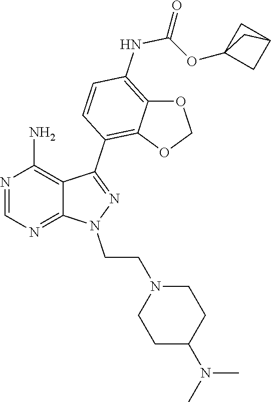 [SMILES: CN(C)C1CCN(CCn2nc(-c3ccc(NC(=O)OC45CC(C4)C5)c4c3OCO4)c3c(N)ncnc32)CC1] | 535 |
| 8 | 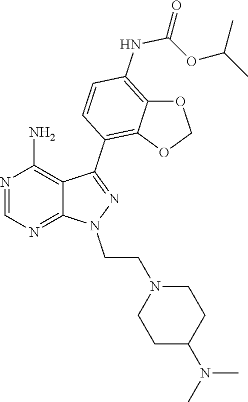 [SMILES: CC(C)OC(=O)Nc1ccc(-c2nn(CCN3CCC(N(C)C)CC3)c3ncnc(N)c23)c2c1OCO2] | 511 |
| 9 | 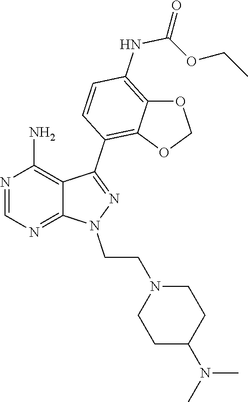 [SMILES: CCOC(=O)Nc1ccc(-c2nn(CCN3CCC(N(C)C)CC3)c3ncnc(N)c23)c2c1OCO2] | 497 |
| 10 | 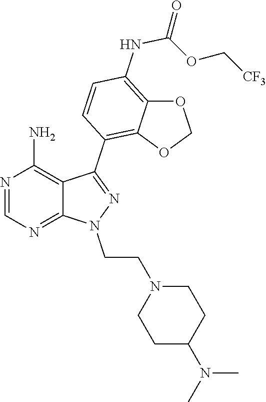 [SMILES: CN(C)C1CCN(CCn2nc(-c3ccc(NC(=O)OCC(F)(F)F)c4c3OCO4)c3c(N)ncnc32)CC1] | 551 |
| 11 | 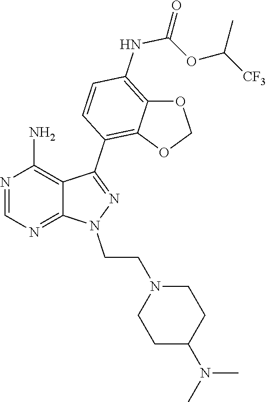 [SMILES: CC(OC(=O)Nc1ccc(-c2nn(CCN3CCC(N(C)C)CC3)c3ncnc(N)c23)c2c1OCO2)C(F)(F)F] | 565 |
| 12 | 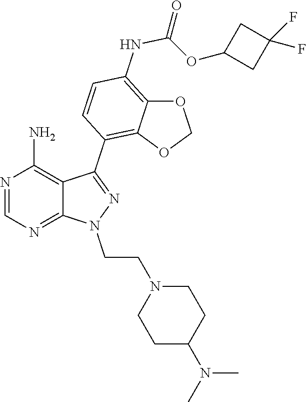 [SMILES: CN(C)C1CCN(CCn2nc(-c3ccc(NC(=O)OC4CC(F)(F)C4)c4c3OCO4)c3c(N)ncnc32)CC1] | 559 |
| 13 | 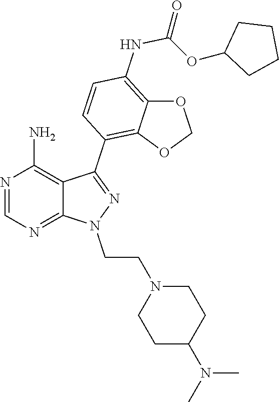 [SMILES: CN(C)C1CCN(CCn2nc(-c3ccc(NC(=O)OC4CCCC4)c4c3OCO4)c3c(N)ncnc32)CC1] | 537 |
| 14 | 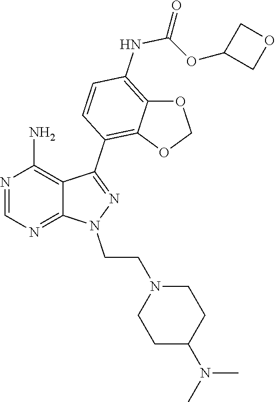 [SMILES: CN(C)C1CCN(CCn2nc(-c3ccc(NC(=O)OC4COC4)c4c3OCO4)c3c(N)ncnc32)CC1] | 525 |
| 15 | 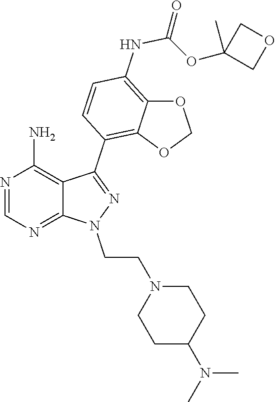 [SMILES: CN(C)C1CCN(CCn2nc(-c3ccc(NC(=O)OC4(C)COC4)c4c3OCO4)c3c(N)ncnc32)CC1] | 539 |
| 16 | 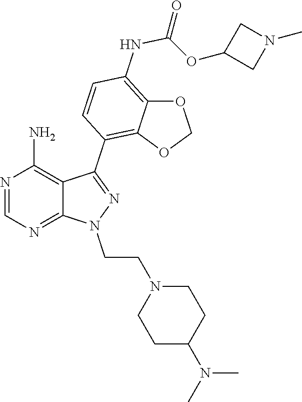 [SMILES: CN1CC(OC(=O)Nc2ccc(-c3nn(CCN4CCC(N(C)C)CC4)c4ncnc(N)c34)c3c2OCO3)C1] | 538 |
| 17 | 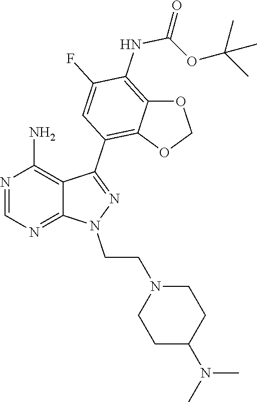 [SMILES: CN(C)C1CCN(CCn2nc(-c3cc(F)c(NC(=O)OC(C)(C)C)c4c3OCO4)c3c(N)ncnc32)CC1] | 543 |
| 18 | 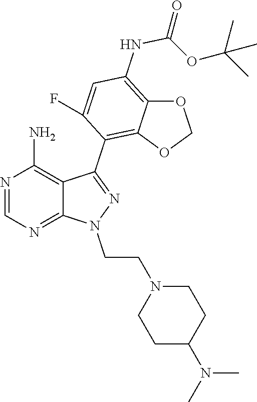 [SMILES: CN(C)C1CCN(CCn2nc(-c3c(F)cc(NC(=O)OC(C)(C)C)c4c3OCO4)c3c(N)ncnc32)CC1] | 543 |
| 19 | 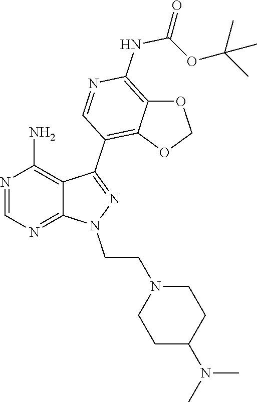 [SMILES: CN(C)C1CCN(CCn2nc(-c3cnc(NC(=O)OC(C)(C)C)c4c3OCO4)c3c(N)ncnc32)CC1] | 526 |
| 20 | 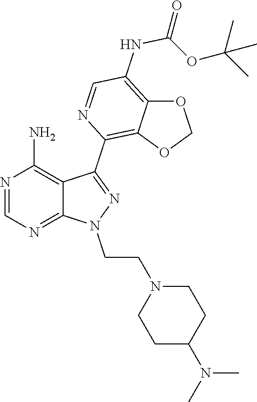 [SMILES: CN(C)C1CCN(CCn2nc(-c3ncc(NC(=O)OC(C)(C)C)c4c3OCO4)c3c(N)ncnc32)CC1] | 526 |
| 21 | 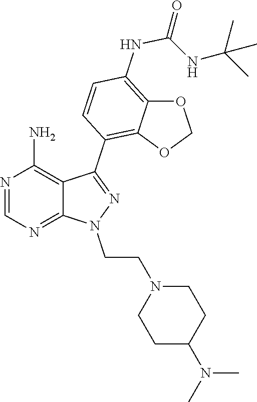 [SMILES: CN(C)C1CCN(CCn2nc(-c3ccc(NC(=O)NC(C)(C)C)c4c3OCO4)c3c(N)ncnc32)CC1] | 524 |
| 22 | 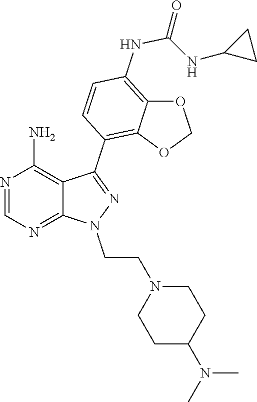 [SMILES: CN(C)C1CCN(CCn2nc(-c3ccc(NC(=O)NC4CC4)c4c3OCO4)c3c(N)ncnc32)CC1] | 508 |
| 23 | 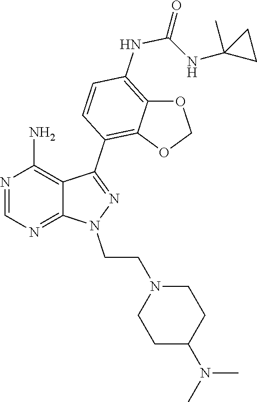 [SMILES: CN(C)C1CCN(CCn2nc(-c3ccc(NC(=O)NC4(C)CC4)c4c3OCO4)c3c(N)ncnc32)CC1] | 522 |
| 24 | 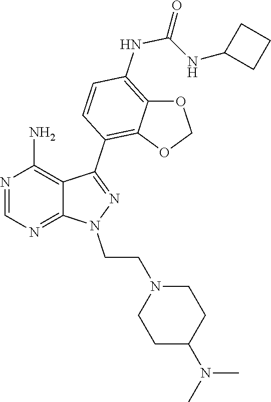 [SMILES: CN(C)C1CCN(CCn2nc(-c3ccc(NC(=O)NC4CCC4)c4c3OCO4)c3c(N)ncnc32)CC1] | 522 |
| 25 | 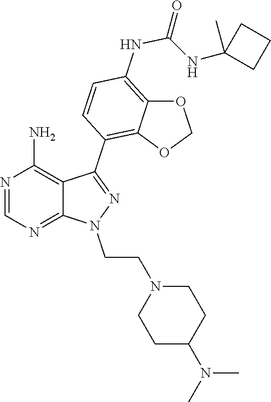 [SMILES: CN(C)C1CCN(CCn2nc(-c3ccc(NC(=O)NC4(C)CCC4)c4c3OCO4)c3c(N)ncnc32)CC1] | 536 |
| 26 | 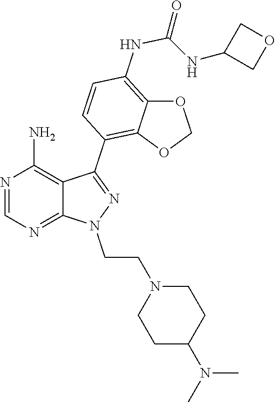 [SMILES: CN(C)C1CCN(CCn2nc(-c3ccc(NC(=O)NC4COC4)c4c3OCO4)c3c(N)ncnc32)CC1] | 524 |
| 27 | 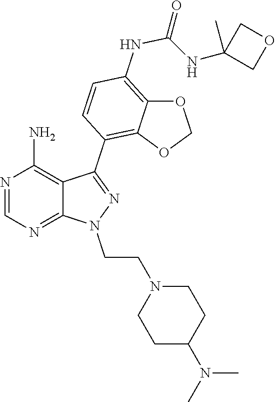 [SMILES: CN(C)C1CCN(CCn2nc(-c3ccc(NC(=O)NC4(C)COC4)c4c3OCO4)c3c(N)ncnc32)CC1] | 538 |
| 28 | 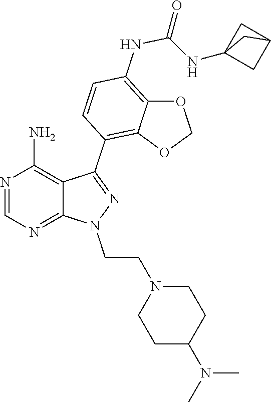 [SMILES: CN(C)C1CCN(CCn2nc(-c3ccc(NC(=O)NC45CC(C4)C5)c4c3OCO4)c3c(N)ncnc32)CC1] | 534 |
| 29 | 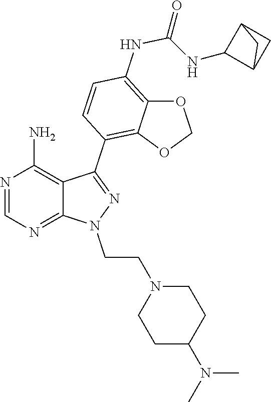 [SMILES: CN(C)C1CCN(CCn2nc(-c3ccc(NC(=O)NC4C5CC4C5)c4c3OCO4)c3c(N)ncnc32)CC1] | 534 |
| 30 | 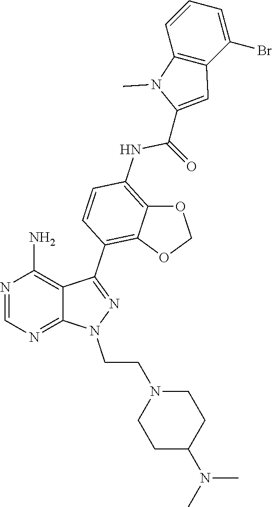 [SMILES: CN(C)C1CCN(CCn2nc(-c3ccc(NC(=O)c4cc5c(Br)cccc5n4C)c4c3OCO4)c3c(N)ncnc32)CC1] | 660 |
| 31 | 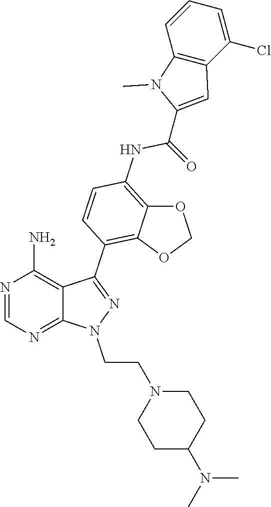 [SMILES: CN(C)C1CCN(CCn2nc(-c3ccc(NC(=O)c4cc5c(Cl)cccc5n4C)c4c3OCO4)c3c(N)ncnc32)CC1] | 616 |
| 32 | 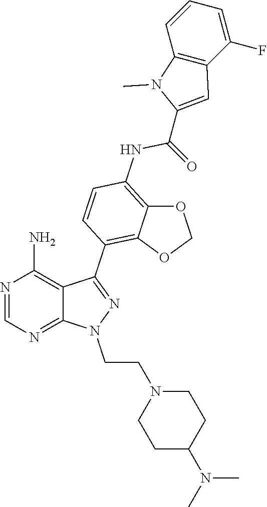 [SMILES: CN(C)C1CCN(CCn2nc(-c3ccc(NC(=O)c4cc5c(F)cccc5n4C)c4c3OCO4)c3c(N)ncnc32)CC1] | 600 |
| 33 | 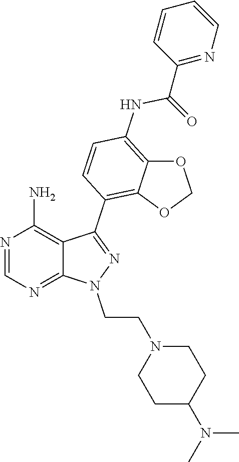 [SMILES: CN(C)C1CCN(CCn2nc(-c3ccc(NC(=O)c4ccccn4)c4c3OCO4)c3c(N)ncnc32)CC1] | 530 |
| 34 | 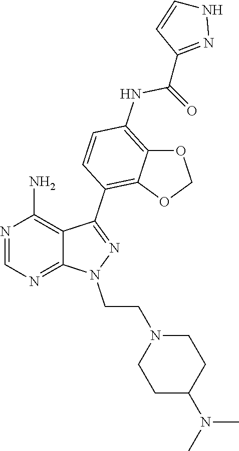 [SMILES: CN(C)C1CCN(CCn2nc(-c3ccc(NC(=O)c4cc[nH]n4)c4c3OCO4)c3c(N)ncnc32)CC1] | 519 |
| 35 | 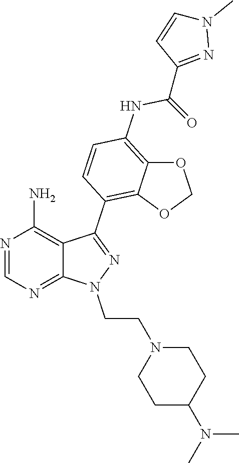 [SMILES: CN(C)C1CCN(CCn2nc(-c3ccc(NC(=O)c4ccn(C)n4)c4c3OCO4)c3c(N)ncnc32)CC1] | 533 |
| 36 | 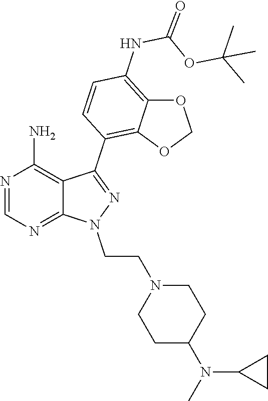 [SMILES: CN(C1CC1)C1CCN(CCn2nc(-c3ccc(NC(=O)OC(C)(C)C)c4c3OCO4)c3c(N)ncnc32)CC1] | 551 |
| 37 | 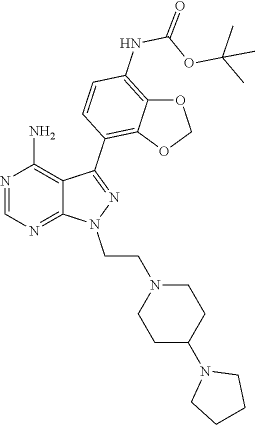 [SMILES: CC(C)(C)OC(=O)Nc1ccc(-c2nn(CCN3CCC(N4CCCC4)CC3)c3ncnc(N)c23)c2c1OCO2] | 551 |
| 38 | 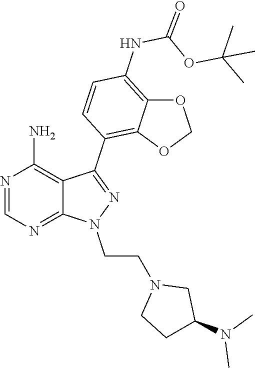 [SMILES: CN(C)[C@H]1CCN(CCn2nc(-c3ccc(NC(=O)OC(C)(C)C)c4c3OCO4)c3c(N)ncnc32)C1] | 511 |
| 39 | 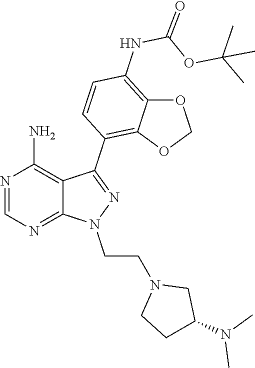 [SMILES: CN(C)[C@@H]1CCN(CCn2nc(-c3ccc(NC(=O)OC(C)(C)C)c4c3OCO4)c3c(N)ncnc32)C1] | 511 |
| 40 | 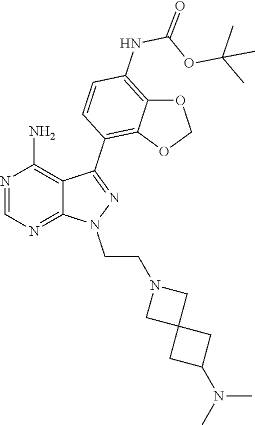 [SMILES: CN(C)C1CC2(C1)CN(CCn1nc(-c3ccc(NC(=O)OC(C)(C)C)c4c3OCO4)c3c(N)ncnc31)C2] | 537 |
| 41 | 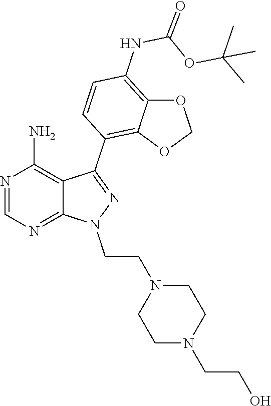 [SMILES: CC(C)(C)OC(=O)Nc1ccc(-c2nn(CCN3CCN(CCO)CC3)c3ncnc(N)c23)c2c1OCO2] | 527 |
| 42 | 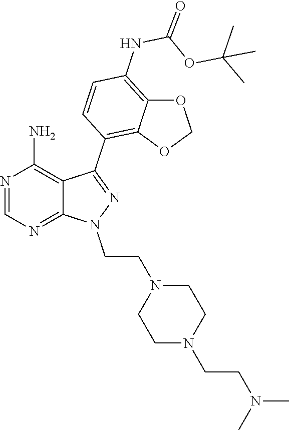 [SMILES: CN(C)CCN1CCN(CCn2nc(-c3ccc(NC(=O)OC(C)(C)C)c4c3OCO4)c3c(N)ncnc32)CC1] | 554 |
| 43 | 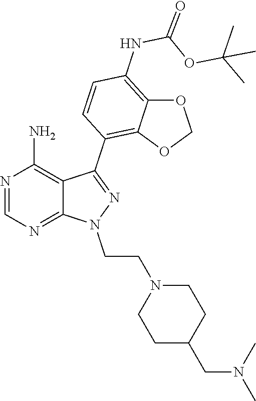 [SMILES: CN(C)CC1CCN(CCn2nc(-c3ccc(NC(=O)OC(C)(C)C)c4c3OCO4)c3c(N)ncnc32)CC1] | 539 |
| 44 | 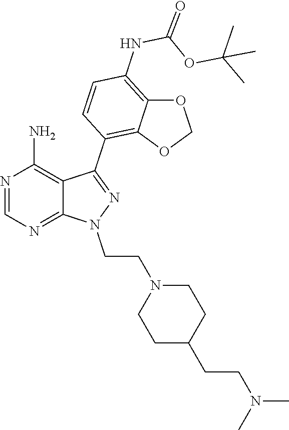 [SMILES: CN(C)CCC1CCN(CCn2nc(-c3ccc(NC(=O)OC(C)(C)C)c4c3OCO4)c3c(N)ncnc32)CC1] | 553 |
| 45 | 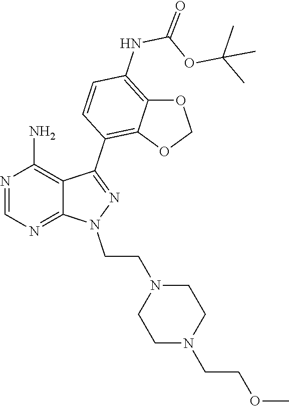 [SMILES: COCCN1CCN(CCn2nc(-c3ccc(NC(=O)OC(C)(C)C)c4c3OCO4)c3c(N)ncnc32)CC1] | 541 |
| 46 | 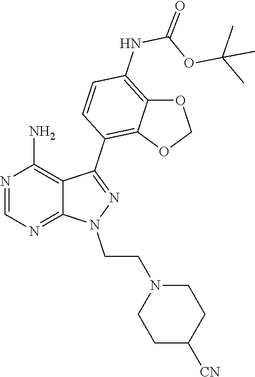 [SMILES: CC(C)(C)OC(=O)Nc1ccc(-c2nn(CCN3CCC(C#N)CC3)c3ncnc(N)c23)c2c1OCO2] | 507 |
| 47 | 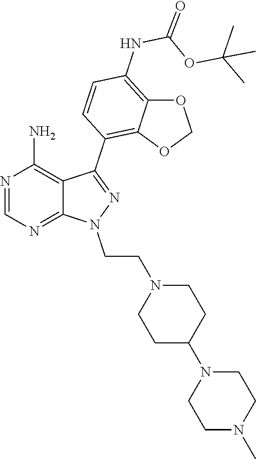 [SMILES: CN1CCN(C2CCN(CCn3nc(-c4ccc(NC(=O)OC(C)(C)C)c5c4OCO5)c4c(N)ncnc43)CC2)CC1] | 580 |
| 48 | 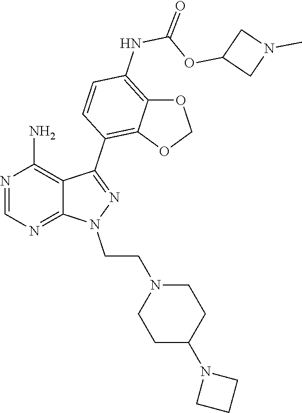 [SMILES: CN1CC(OC(=O)Nc2ccc(-c3nn(CCN4CCC(N5CCC5)CC4)c4ncnc(N)c34)c3c2OCO3)C1] | 550 |
| 49 | 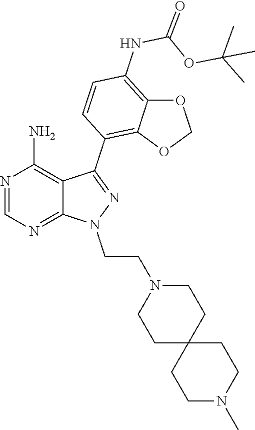 [SMILES: CN1CCC2(CC1)CCN(CCn1nc(-c3ccc(NC(=O)OC(C)(C)C)c4c3OCO4)c3c(N)ncnc31)CC2] | 565 |
| 50 | 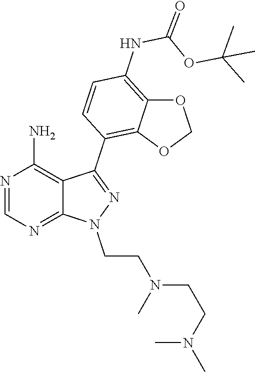 [SMILES: CN(C)CCN(C)CCn1nc(-c2ccc(NC(=O)OC(C)(C)C)c3c2OCO3)c2c(N)ncnc21] | 499 |
| 51 | 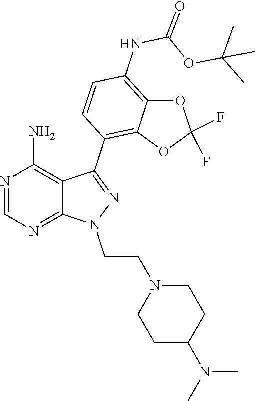 [SMILES: CN(C)C1CCN(CCn2nc(-c3ccc(NC(=O)OC(C)(C)C)c4c3OC(F)(F)O4)c3c(N)ncnc32)CC1] | 561 |
| 52 | 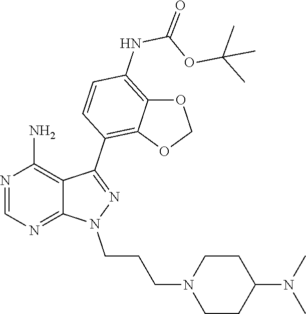 [SMILES: CN(C)C1CCN(CCCn2nc(-c3ccc(NC(=O)OC(C)(C)C)c4c3OCO4)c3c(N)ncnc32)CC1] | 539 |
| 53 | 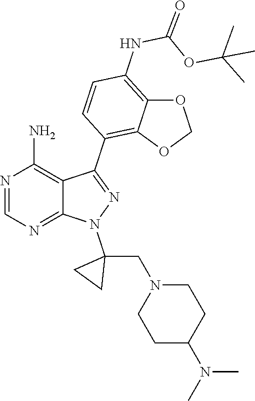 [SMILES: CN(C)C1CCN(CC2(n3nc(-c4ccc(NC(=O)OC(C)(C)C)c5c4OCO5)c4c(N)ncnc43)CC2)CC1] | 551 |
| 54 | 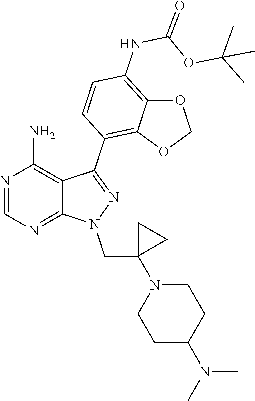 [SMILES: CN(C)C1CCN(C2(Cn3nc(-c4ccc(NC(=O)OC(C)(C)C)c5c4OCO5)c4c(N)ncnc43)CC2)CC1] | 551 |
| 55 | 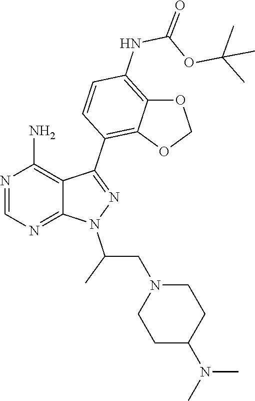 [SMILES: CC(CN1CCC(N(C)C)CC1)n1nc(-c2ccc(NC(=O)OC(C)(C)C)c3c2OCO3)c2c(N)ncnc21] | 539 |
| 56 | 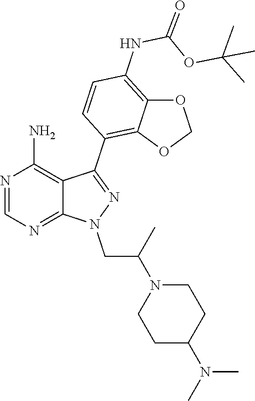 [SMILES: CC(Cn1nc(-c2ccc(NC(=O)OC(C)(C)C)c3c2OCO3)c2c(N)ncnc21)N1CCC(N(C)C)CC1] | 539 |
| 57 | 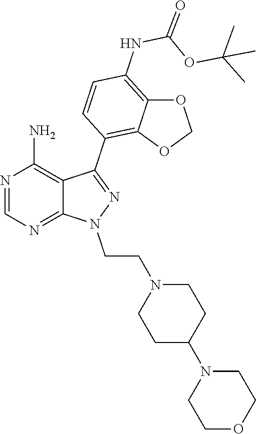 [SMILES: CC(C)(C)OC(=O)Nc1ccc(-c2nn(CCN3CCC(N4CCOCC4)CC3)c3ncnc(N)c23)c2c1OCO2] | 567 |
| 58 | 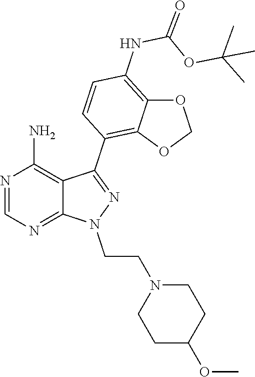 [SMILES: COC1CCN(CCn2nc(-c3ccc(NC(=O)OC(C)(C)C)c4c3OCO4)c3c(N)ncnc32)CC1] | 512 |

### Example 59. Synthesis of Compound 59

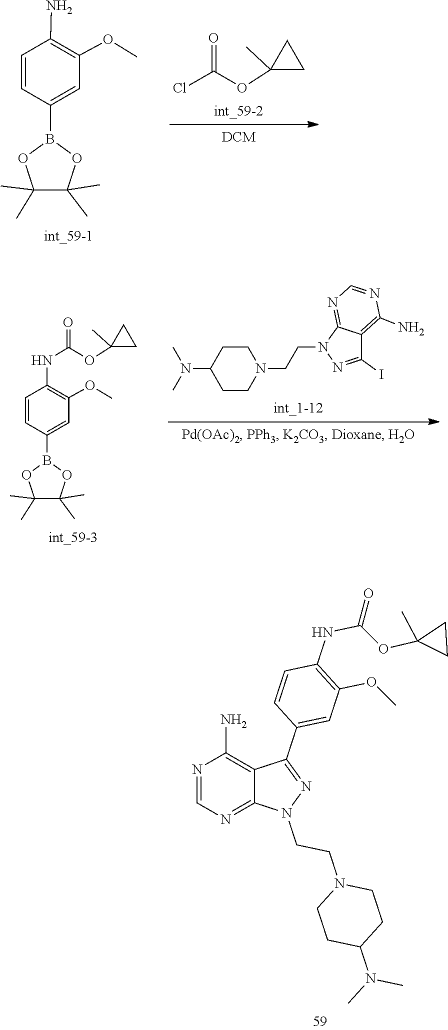

### Step 1: Synthesis of Compound Int 59-3

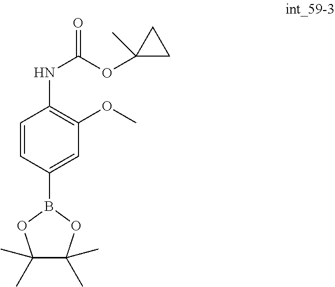 [SMILES: COc1cc(B2OC(C)(C)C(C)(C)O2)ccc1NC(=O)OC1(C)CC1]

Int_59-2 (300 mg, 2.22 mmol) was dissolved in dichloromethane (5 mL), and int_59-1 (186 mg, 0.74 mmol) and DIPEA (581.6 mg, 4.5 mmol) were added to the reaction solution at 0° C. After the addition was completed, the reaction solution was stirred at room temperature for 16 h. LC-MS monitoring showed the reaction was completed. Water (10 mL) was added to the reaction solution. The aqueous phase was extracted with ethyl acetate (10 mL×3), and the organic phase was dried over anhydrous sodium sulfate. The organic phase was concentrated under reduced pressure to give a crude product. The crude product was purified by preparative column chromatography (SiO₂, DCM/MeOH=98/2) to give a solid (35 mg, 4.5% yield).

### Step 2: Synthesis of Compound 59

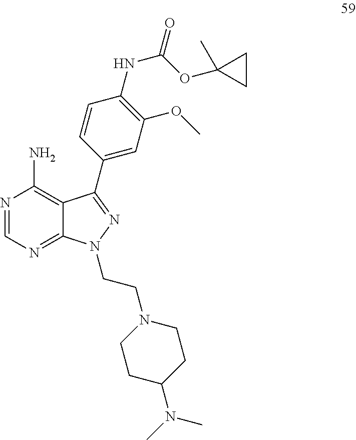 [SMILES: COc1cc(-c2nn(CCN3CCC(N(C)C)CC3)c3ncnc(N)c23)ccc1NC(=O)OC1(C)CC1]

Int_59-3 (30 mg, 0.086 mmol) and int_1-12 (54 mg, 0.130 mmol) were dissolved in Dioxane (3 mL) and H₂O (0.5 mL). Palladium acetate (1 mg, 0.004 mmol), PPh₃ (45 mg, 0.173 mmol) and potassium carbonate (24 mg, 0.173 mmol) were added to the reaction solution. The reaction solution was microwaved to 120° C. and reacted for 0.5 h under nitrogen atmosphere. LC-MS monitoring showed the reaction was completed. The reaction solution was concentrated under reduced pressure to give a crude product. The crude product was purified by preparative column chromatography (SiO₂, DCM/MeOH=90/10) and then purified by preparative HPLC to give a solid (5 mg, 11.4% yield).

¹H NMR (400 MHz, DMSO-d6) δ 8.38 (s, 1H), 8.21 (d, J=5.9 Hz, 1H), 7.87 (d, J=8.1 Hz, 1H), 7.20 (d, J=8.5 Hz, 2H), 4.41 (t, J=6.8 Hz, 2H), 3.84 (s, 3H), 2.93 (s, 2H), 2.75 (d, J=6.9 Hz, 2H), 2.17 (s, 6H), 2.00-1.90 (m, 3H), 1.68 (d, J=12.3 Hz, 2H), 1.52 (s, 3H), 1.26 (dd, J=12.0, 3.6 Hz, 2H), 0.88-0.83 (m, 2H), 0.68-0.62 (m, 2H).

MS (ESI): 509 [M+H]⁺.

### Example 60. Synthesis of Compound 60

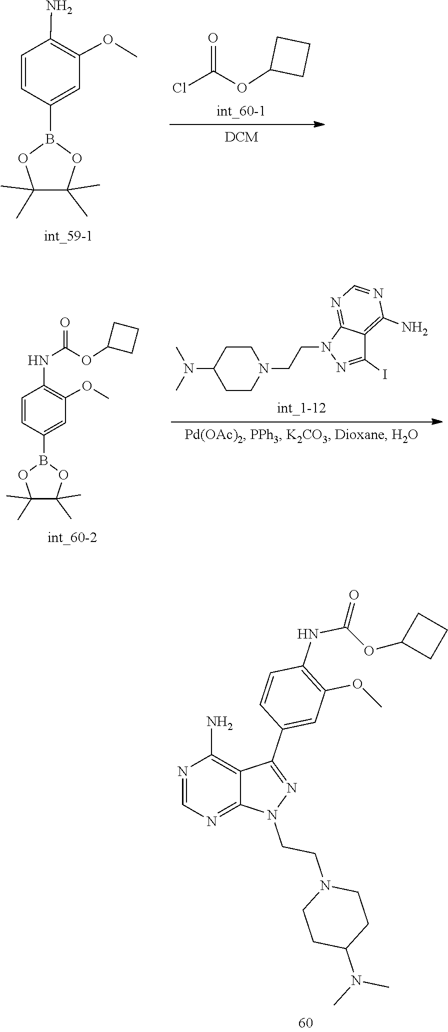

### Step 1: Synthesis of Compound Int 60-2

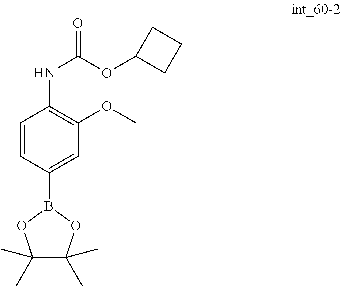 [SMILES: COc1cc(B2OC(C)(C)C(C)(C)O2)ccc1NC(=O)OC1CCC1]

Int_60-1 (200 mg, 1.486 mmol) was dissolved in dichloromethane (5 mL), and int_59-1 (325 mg, 1.341 mmol) and DIPEA (550 mg, 4.245 mmol) were added to the reaction solution at 0° C. After the addition was completed, the reaction solution was stirred at room temperature for 16 h. LC-MS monitoring showed the reaction was completed. Water (10 mL) was added to the reaction solution. The aqueous phase was extracted with ethyl acetate (10 mL×3), and the organic phase was dried over anhydrous sodium sulfate. The organic phase was concentrated under reduced pressure to give a crude product. The crude product was purified by preparative column chromatography (SiO₂, DCM/MeOH=98/2) to give a solid (106 mg, 22.7% yield).

ESI-MS m/z: 348 [M+H]⁺.

### Step 2: Synthesis of Compound 60

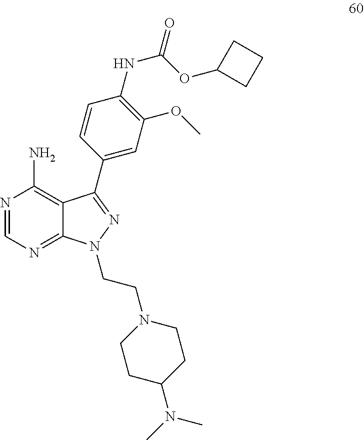 [SMILES: COc1cc(-c2nn(CCN3CCC(N(C)C)CC3)c3ncnc(N)c23)ccc1NC(=O)OC1CCC1]

Int_60-2 (100 mg, 0.288 mmol) and int_1-12 (120 mg, 0.288 mmol) were dissolved in Dioxane (3 mL) and H₂O (0.5 mL). Palladium acetate (4 mg, 0.014 mmol), PPh₃ (151 mg, 0.576 mmol) and potassium carbonate (80 mg, 0.576 mmol) were added to the reaction solution. The reaction solution was microwaved to 120° C. and reacted for 0.5 h under nitrogen atmosphere. LC-MS monitoring showed the reaction was completed. The reaction solution was concentrated under reduced pressure to give a crude product. The crude product was purified by preparative column chromatography (SiO₂, DCM/MeOH=90/10) and then purified by preparative HPLC to give a solid (25 mg, 17% yield).

¹H NMR (400 MHz, DMSO-d6) δ 8.47 (s, 1H), 8.22 (s, 1H), 7.84 (d, J=8.1 Hz, 1H), 7.23-7.16 (m, 2H), 4.92 (p, J=7.5 Hz, 1H), 4.41 (t, J=6.8 Hz, 2H), 3.86 (s, 3H), 2.93 (d, J=11.0 Hz, 2H), 2.74 (t, J=6.8 Hz, 2H), 2.28 (dddt, J=9.8, 7.6, 5.3, 2.6 Hz, 2H), 2.12 (s, 6H), 2.09-1.89 (m, 5H), 1.77-1.55 (m, 4H), 1.25 (tt, J=12.1, 6.1 Hz, 2H).

MS (ESI): 509 [M+H]⁺.

### Example 61. Synthesis of Compound 61

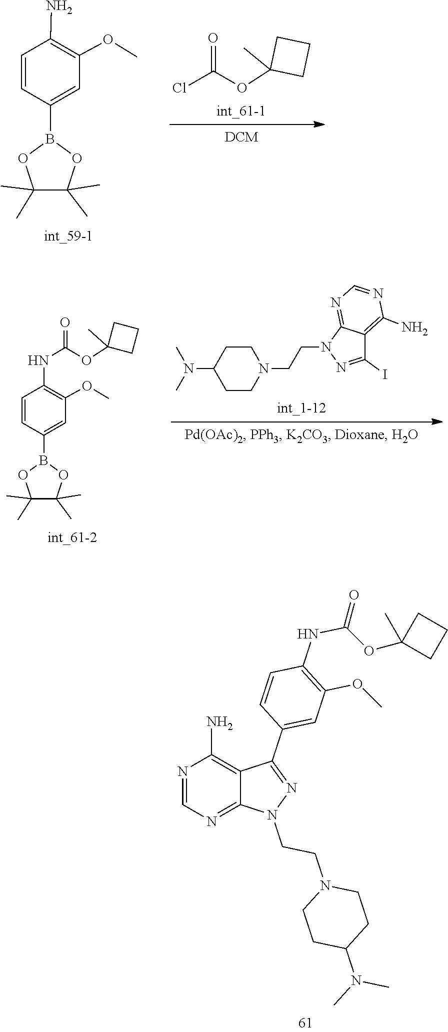

### Step 1: Synthesis of Compound Int_61-2

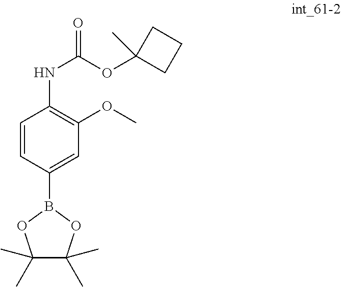 [SMILES: COc1cc(B2OC(C)(C)C(C)(C)O2)ccc1NC(=O)OC1(C)CCC1]

Int_61-1 (150 mg, 1.00 mmol) was dissolved in dichloromethane (5 mL), and int_59-1 (125 mg, 0.505 mmol) and DIPEA (258.4 mg, 2 mmol) were added to the reaction solution at 0° C. After the addition was completed, the reaction solution was stirred at room temperature for 16 h. LC-MS monitoring showed the reaction was completed. Water (10 mL) was added to the reaction solution. The aqueous phase was extracted with ethyl acetate (10 mL×3), and the organic phase was dried over anhydrous sodium sulfate. The organic phase was concentrated under reduced pressure to give a crude product. The crude product was purified by preparative column chromatography (SiO₂, DCM/MeOH=98/2) to give a solid (95 mg, 52% yield).

ESI-MS m/z: 362 [M+H]⁺.

### Step 2: Synthesis of Compound 61

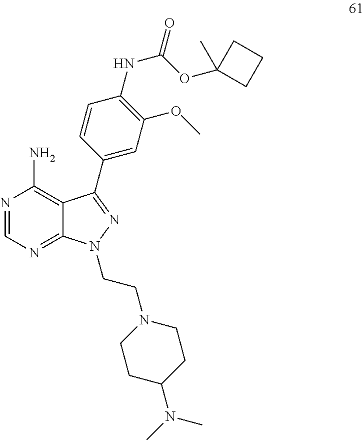 [SMILES: COc1cc(-c2nn(CCN3CCC(N(C)C)CC3)c3ncnc(N)c23)ccc1NC(=O)OC1(C)CCC1]

Int_61-2 (95 mg, 0.263 mmol) and int_1-12 (163 mg, 0.394 mmol) were dissolved in Dioxane (3 mL) and H₂O (0.5 mL). Palladium acetate (3 mg, 0.013 mmol), PPh₃ (138 mg, 0.526 mmol) and potassium carbonate (73 mg, 0.526 mmol) were added to the reaction solution. The reaction solution was microwaved to 120° C. and reacted for 0.5 h under nitrogen atmosphere. LC-MS monitoring showed the reaction was completed. The reaction solution was concentrated under reduced pressure to give a crude product. The crude product was purified by preparative column chromatography (SiO₂, DCM/MeOH=90/10) and then purified by preparative HPLC to give a solid (49 mg, 35.6% yield).

¹H NMR (400 MHz, DMSO-d6) δ 8.27 (s, 1H), 8.22 (s, 1H), 7.87 (d, J=8.2 Hz, 1H), 7.23-7.15 (m, 2H), 4.41 (t, J=6.8 Hz, 2H), 3.86 (s, 3H), 2.95 (d, J=10.8 Hz, 2H), 2.75 (t, J=6.8 Hz, 2H), 2.31 (dt, J=12.3, 9.4 Hz, 2H), 2.18 (s, 6H), 2.08 (dtd, J=9.5, 8.1, 4.0 Hz, 3H), 1.95 (td, J=11.6, 2.3 Hz, 2H), 1.82-1.61 (m, 4H), 1.55 (s, 3H), 1.28 (qd, J=12.0, 3.7 Hz, 2H).

MS (ESI): 523 [M+H]⁺.

### Example 62. Synthesis of Compound 62

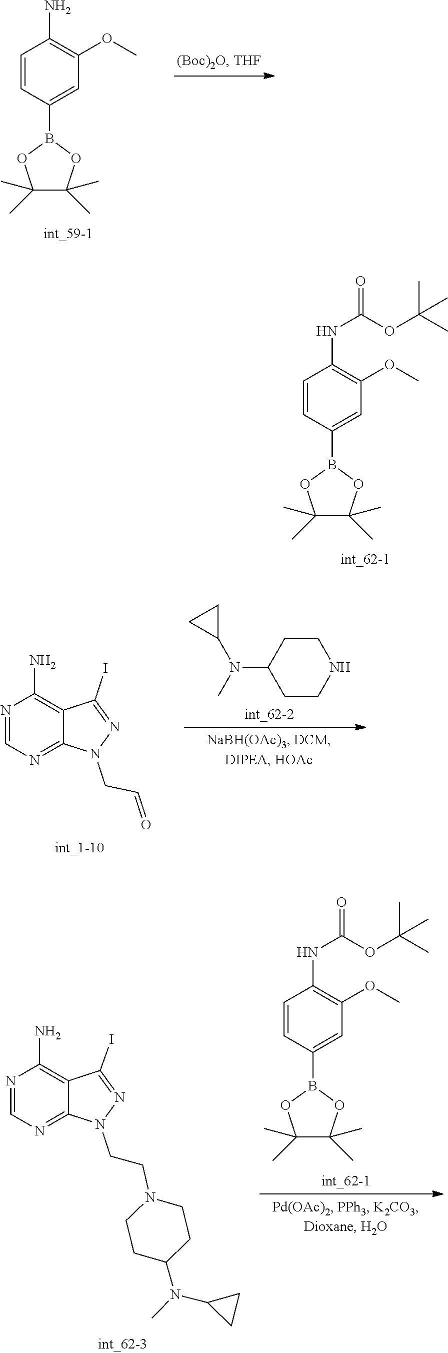

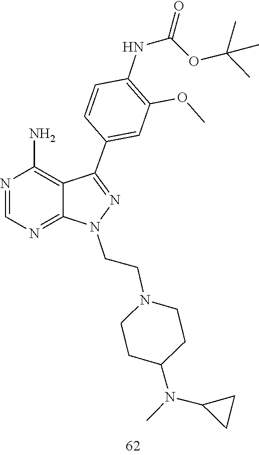 [SMILES: COc1cc(-c2nn(CCN3CCC(N(C)C4CC4)CC3)c3ncnc(N)c23)ccc1NC(=O)OC(C)(C)C]

### Step 1: Synthesis of Compound Int_62-2

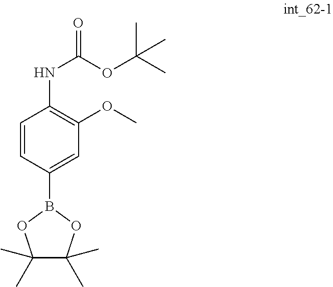 [SMILES: COc1cc(B2OC(C)(C)C(C)(C)O2)ccc1NC(=O)OC(C)(C)C]

Int_59-1 (450 mg, 1.80 mmol) was dissolved in THE (20 mL), and (Boc)₂O (788 mg, 3.613 mmol) was added at room temperature. The reaction solution was reacted at 80° C. for 16 h. LC-MS monitoring showed the reaction was completed. The reaction solution was concentrated under reduced pressure to remove THE to give a crude product (500 mg), which was directly used in the next step.

ESI-MS m/z: 350, 295 [M+H]⁺.

### Step 2: Synthesis of Compound Int 62-3

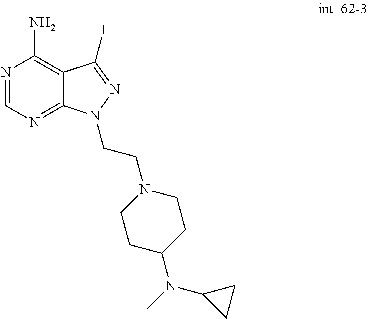 [SMILES: CN(C1CC1)C1CCN(CCn2nc(I)c3c(N)ncnc32)CC1]

Int_1-10 (100 mg, 0.33 mmol) and int_62-2 (80 mg, 0.49 mmol) were dissolved in dichloromethane (10 mL). Sodium triacetoxyborohydride (280 mg, 1.32 mmol), DIPEA (170.6 mg, 1.32 mmol) and acetic acid (79.2 mg, 1.32 mmol) were added to the reaction solution. The reaction solution was reacted at room temperature for 16 h. LC-MS monitoring showed the reaction was completed. Water (20 mL) was slowly added to the reaction solution. The aqueous phase was extracted with dichloromethane (20 mL×3), and the organic phase was dried over anhydrous sodium sulfate. The organic phase was concentrated under reduced pressure to give a crude product. The crude product was purified by preparative column chromatography (SiO₂, DCM/MeOH=90/10) to give a solid (120 mg, 82.4% yield).

MS (ESI): 442 [M+H]⁺.

### Step 3: Synthesis of Compound 62

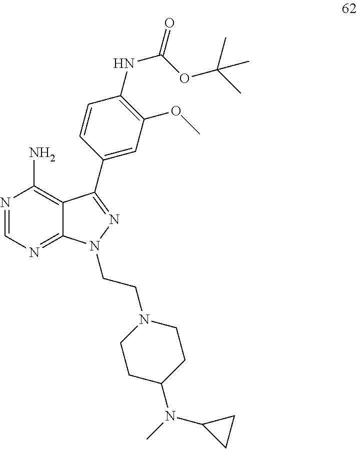 [SMILES: COc1cc(-c2nn(CCN3CCC(N(C)C4CC4)CC3)c3ncnc(N)c23)ccc1NC(=O)OC(C)(C)C]

Int_62-1 (50 mg, 0.143 mmol) and int_62-3 (94 mg, 0.215 mmol) were dissolved in Dioxane (3 mL) and H₂O (0.5 mL). Palladium acetate (1.5 mg, 0.007 mmol), PPh₃ (75 mg, 0.286 mmol) and potassium carbonate (40 mg, 0.286 mmol) were added to the reaction solution. The reaction solution was microwaved to 120° C. and reacted for 0.5 h under nitrogen atmosphere. LC-MS monitoring showed the reaction was completed. The reaction solution was concentrated under reduced pressure to give a crude product. The crude product was purified by preparative column chromatography (SiO₂, DCM/MeOH=90/10) and then purified by preparative HPLC to give a solid (8 mg, 10.4% yield).

¹H NMR (400 MHz, DMSO-d6) δ 8.22 (s, 1H), 8.03 (s, 1H), 7.89 (d, J=8.2 Hz, 1H), 7.23-7.18 (m, 2H), 4.41 (t, J=6.8 Hz, 2H), 3.86 (s, 3H), 2.96 (d, J=10.9 Hz, 2H), 2.74 (t, J=6.9 Hz, 2H), 2.30 (t, J=11.5 Hz, 1H), 2.18 (s, 3H), 1.95 (t, J=11.3 Hz, 2H), 1.74-1.63 (m, 3H), 1.46 (s, 9H), 1.41-1.29 (m, 2H), 0.43-0.36 (m, 2H), 0.27-0.18 (m, 2H).

MS (ESI): 537 [M+H]⁺.

### Example 63. Synthesis of Compound 69

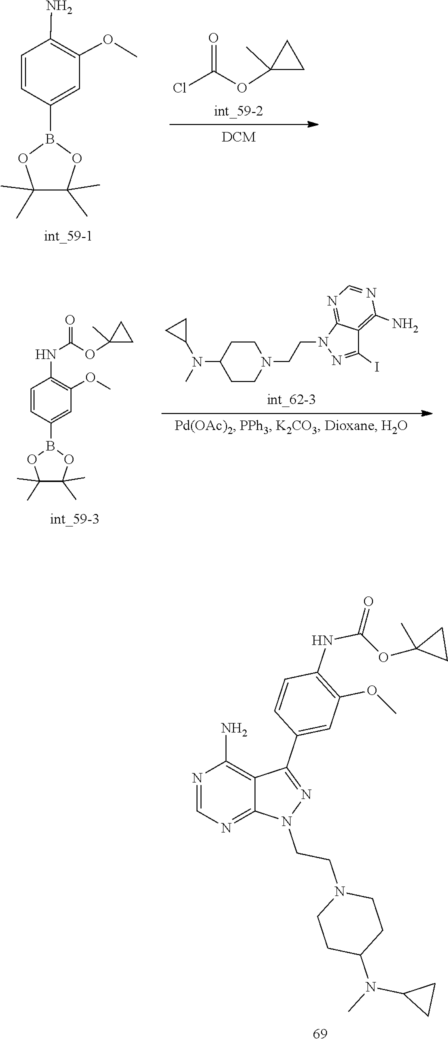

### Step 1: Synthesis of Compound Int_59-3

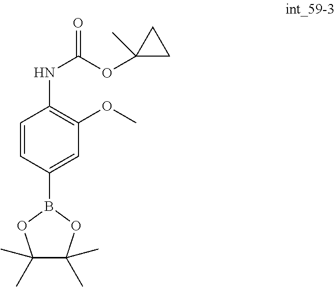 [SMILES: COc1cc(B2OC(C)(C)C(C)(C)O2)ccc1NC(=O)OC1(C)CC1]

Int_59-2 (300 mg, 2.22 mmol) was dissolved in dichloromethane (5 mL), and int_59-1 (186 mg, 0.74 mmol) and DIPEA (581.6 mg, 4.5 mmol) were added to the reaction solution at 0° C. After the addition was completed, the reaction solution was stirred at room temperature for 16 h. LC-MS monitoring showed the reaction was completed. Water (10 mL) was added to the reaction solution. The aqueous phase was extracted with ethyl acetate (10 mL×3), and the organic phase was dried over anhydrous sodium sulfate. The organic phase was concentrated under reduced pressure to give a crude product. The crude product was purified by preparative column chromatography (SiO₂, DCM/MeOH=98/2) to give a solid (35 mg, 4.5% yield).

ESI-MS m/z: 348 [M+H]⁺.

### Step 2: Synthesis of Compound 69

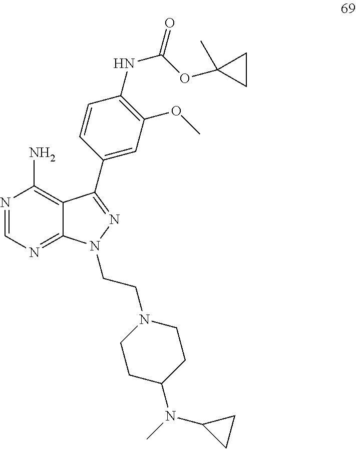 [SMILES: COc1cc(-c2nn(CCN3CCC(N(C)C4CC4)CC3)c3ncnc(N)c23)ccc1NC(=O)OC1(C)CC1]

Int_59-3 (50 mg, 0.144 mmol) and int_62-3 (64 mg, 0.144 mmol) were dissolved in Dioxane (3 mL) and H₂O (0.5 mL). Palladium acetate (3 mg, 0.014 mmol), PPh₃ (75 mg, 0.288 mmol) and potassium carbonate (60 mg, 0.432 mmol) were added to the reaction solution. The reaction solution was microwaved to 120° C. and reacted for 2 h under nitrogen atmosphere. LC-MS monitoring showed the reaction was completed. The reaction solution was concentrated under reduced pressure to give a crude product. The crude product was purified by preparative column chromatography (SiO₂, DCM/MeOH=90/10) and then purified by preparative HPLC to give a solid (4 mg, 5.2% yield).

¹H NMR (400 MHz, DMSO-d6) δ 8.42 (d, J=7.0 Hz, 1H), 8.22 (s, 1H), 8.16 (s, 1H), 7.86 (d, J=8.1 Hz, 1H), 7.20 (d, J=8.0 Hz, 2H), 4.41 (t, J=6.5 Hz, 2H), 3.84 (s, 3H), 2.96 (d, J=10.4 Hz, 2H), 2.75 (q, J=7.3 Hz, 3H), 2.30 (s, 1H), 2.15 (d, J=23.4 Hz, 3H), 1.96 (d, J=12.9 Hz, 2H), 1.74-1.65 (m, 2H), 1.52 (s, 3H), 1.21 (s, 2H), 0.84 (d, J=6.4 Hz, 2H), 0.68-0.63 (m, 2H), 0.38 (dd, J=6.8, 4.7 Hz, 2H), 0.25-0.16 (in, 2H).

MS (ESI): 535 [M+H]⁺.

### Examples 64-70. Synthesis of Compounds 64-70

The target compounds 63-68 and compound 70 in Table 2 could be obtained by the synthesis methods described above using different starting materials.

TABLE 2

| | | MS |
|---|---|---|
| Compound | Compound Structure | (M + H)⁺ |
| 63 | 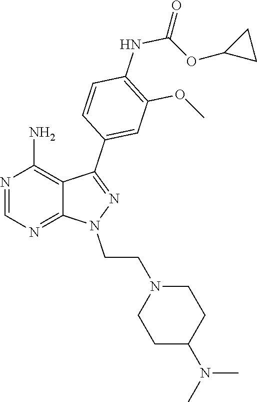 [SMILES: COc1cc(-c2nn(CCN3CCC(N(C)C)CC3)c3ncnc(N)c23)ccc1NC(=O)OC1CC1] | 495 |
| 64 | 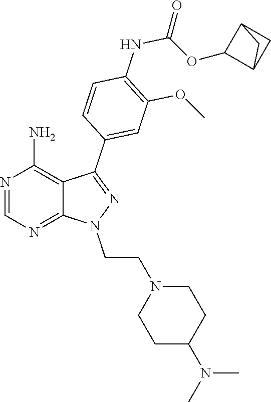 [SMILES: COc1cc(-c2nn(CCN3CCC(N(C)C)CC3)c3ncnc(N)c23)ccc1NC(=O)OC1C2CC1C2] | 521 |
| 65 | 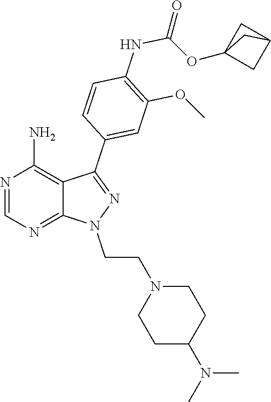 [SMILES: COc1cc(-c2nn(CCN3CCC(N(C)C)CC3)c3ncnc(N)c23)ccc1NC(=O)OC12CC(C1)C2] | 521 |
| 66 | 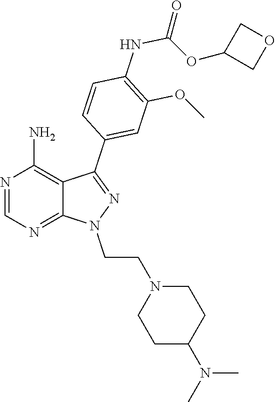 [SMILES: COc1cc(-c2nn(CCN3CCC(N(C)C)CC3)c3ncnc(N)c23)ccc1NC(=O)OC1COC1] | 511 |
| 67 | 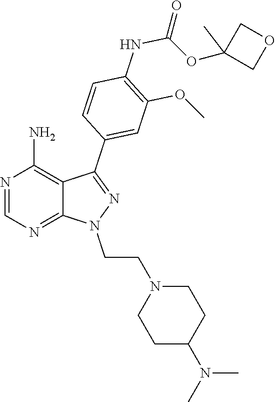 [SMILES: COc1cc(-c2nn(CCN3CCC(N(C)C)CC3)c3ncnc(N)c23)ccc1NC(=O)OC1(C)COC1] | 525 |
| 68 | 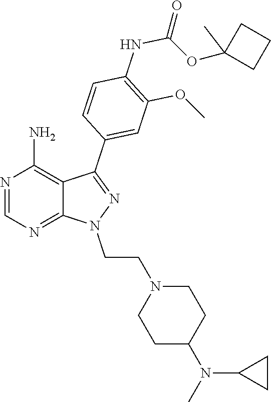 [SMILES: COc1cc(-c2nn(CCN3CCC(N(C)C4CC4)CC3)c3ncnc(N)c23)ccc1NC(=O)OC1(C)CCC1] | 549 |
| 69 | 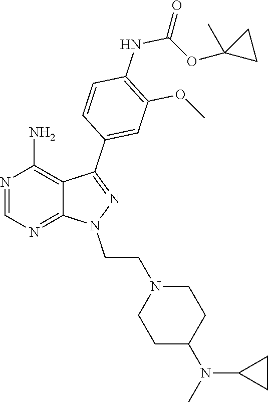 [SMILES: COc1cc(-c2nn(CCN3CCC(N(C)C4CC4)CC3)c3ncnc(N)c23)ccc1NC(=O)OC1(C)CC1] | 535 |
| 70 | 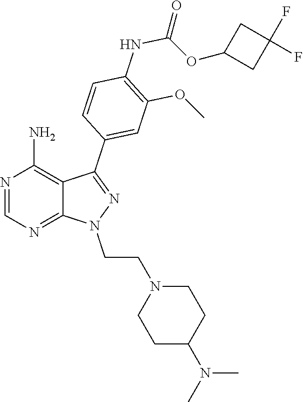 [SMILES: COc1cc(-c2nn(CCN3CCC(N(C)C)CC3)c3ncnc(N)c23)ccc1NC(=O)OC1CC(F)(F)C1] | 545 |

### Example 71. In Vitro Assay of Compounds of the Present Invention for Inhibiting Src Kinase Activity

After the compound serially diluted with DMSO and a Src recombinant protein were incubated at room temperature for 10 min, the reaction was initiated by the addition of a biotin-labeled TK substrate and ATP. After the mixture was left to stand at room temperature for 40 min Sa-XL 665 and a Cryptate-labeled antibody were added. The fluorescence values at 615 nm and 665 nm were measured, and the ratio of 665 nm to 615 nm was calculated. The inhibition percentages and IC₅₀ of the compounds were calculated compared to the DMSO group. The results are shown in Table 3 below.

TABLE 3
Inhibitory activity of compounds of the present
invention against recombinant protein Src (IC₅₀, nM)

| Compound | IC₅₀ |
|---|---|
| 1 | +++ |
| 2 | +++ |
| 3 | +++ |
| 4 | +++ |
| 5 | +++ |
| 6 | +++ |
| 7 | +++ |
| 8 | ++ |
| 9 | ++ |
| 12 | +++ |
| 13 | ++ |
| 14 | +++ |
| 17 | ++ |
| 18 | ++ |
| 19 | ++ |
| 20 | ++ |
| 36 | +++ |
| 37 | +++ |
| 38 | +++ |
| 39 | +++ |
| 41 | +++ |
| 42 | +++ |
| 43 | +++ |
| 44 | +++ |
| 45 | +++ |
| 46 | +++ |
| 47 | +++ |
| 48 | +++ |
| 49 | +++ |
| 50 | +++ |
| 51 | +++ |
| 52 | +++ |
| 57 | +++ |
| 58 | +++ |
| 59 | +++ |
| 60 | +++ |
| 61 | +++ |
| 62 | +++ |
| 63 | +++ |
| 64 | +++ |
| 65 | +++ |
| 68 | +++ |
| 69 | +++ |
| 70 | +++ |

+++ means that IC₅₀ is less than or equal to 10 nM
++ means that IC₅₀ is 10 nM to 50 nM
+ means that IC₅₀ is greater than 50 nM.

### Example 72. In Vitro Anti-Proliferative Activity of Compounds of the Present Invention Against PC3 Cells

PC3 cells were mixed with 0.4% agarose at 1200 cells/well, and the mixture was seeded on 0.6% agarose. The compound serially diluted was added, and the cell number was measured after 7 days of incubation. The inhibition percentages and IC₅₀ of the compounds were calculated compared to the DMSO group. The results are shown in Table 4 below.

TABLE 4
In Vitro anti-proliferative activity of compounds of
the present invention against PC3 cells (IC₅₀, nM)

| Compound | IC₅₀ (nM) | Compound | IC₅₀ (nM) |
|---|---|---|---|
| 1 | 3.5 | 3 | 4.8 |
| 5 | 4.2 | 37 | 11.7 |
| 59 | 5.3 | 61 | 5.6 |
| 62 | 10.1 | 68 | 15.2 |
| 69 | 12.5 | | |

As can be seen from the data in Table 4, the compounds of the present invention have unexpectedly strong in vitro anti-proliferative activity against PC3 cells.

### Example 73. In Vivo Pharmacodynamic Study—Mouse PC3 Subcutaneous Xenograft Tumor Model

Each nude mouse was grafted subcutaneously with 10×10⁶ PC3 cells. When the tumor grew to 100-200 mm³, the compound was administered orally once a day, and the tumor volume was measured twice a week and at the end of treatment. Tumor growth inhibition rate of the compound was calculated according to the following equation: tumor growth inhibition (TGI)=1−(tumor volume on day 17 in treatment group−tumor volume on day 1 in treatment group)/(tumor volume on day 17 in vehicle control group−tumor volume on day 1 in treatment group). The results are shown in Table 5.

TABLE 5
Growth inhibition in mouse PC3 subcutaneous xenograft tumor

| | | Tumor | Tumor | |
|---|---|---|---|---|
| | | volume on | volume on | |
| | | day 1 of | day 17 of | |
| | | treatment | treatment | |
| Compound | Dose | (mm³) | (mm³) | TGI |
| Control | Not | 120 | 540 | Not |
| | applicable | | | applicable |
| Compound | 50 mg/kg(day | 120 | 318 | 53% |
| 59 | 1-day 7), 80 | | | |
| | mg/kg(day 8- | | | |
| | day 18) | | | |

As can be seen from Table 5, the compound of the present invention was able to inhibit tumor growth in the mouse PC3 subcutaneous xenograft tumor model.

### Example 74. In Vivo Pharmacokinetic Experiment of Compound of the Present Invention

CD-1 female mice aged 7 to 10 weeks were intravenously administered and orally administered at a dose of 2 mg/kg and 10 mg/kg, respectively. The mice were fasted for at least 12 h before the administration and given food 4 h after the administration, and they were given ad libitum access to water during the experiment. On the day of the experiment, animals in the intravenous group were administered the corresponding compound by single injection via the tail vein at an administration volume of 10 mL/kg. Animals in the oral group were administered the corresponding compound by single intragastric injection at an administration volume of 10 mL/kg. The animals were weighed before administration, and the administration volume was calculated according to the body weight. The sample collection time was 0.083 h (injection group), 0.167 h, 0.5 h, 1 h, 2 h, 4 h, 8 h, and 24 h. About 200 μL of whole blood was collected through the submaxillary venous plexus at each time point and used to prepare plasma for concentration determination by high-performance liquid chromatography-tandem mass spectrometry (LC-MS/MS). All animals were euthanized with CO₂ anesthesia after the PK samples were collected at the last time point. The plasma concentrations were processed using a non-compartmental model of Phoenix WinNonlin™ version 8.3 (Certara) pharmacokinetic software, and the pharmacokinetic parameters were calculated using a linear-log trapezoidal method. The in vivo pharmacokinetic results are shown in Table 6 below.

TABLE 6
Results of in vivo pharmacokinetic evaluation
of compound of the present invention

| Route of | Pharmacokinetic | | |
|---|---|---|---|
| administration | parameters | 59 | Compound A |
| Injection | Vdss (L/Kg) | 35.6 | 15.2 |
| (2 mg/kg) | T_{1/2} (h) | 5.9 | 6.4 |
| | Cl (L/h/Kg) | 105.9 | 46.5 |
| | AUC₀₋ₗₐₛₜ (h*ng/mL) | 299.9 | 681.5 |
| Oral | Cₘₐₓ (ng/mL) | 88.9 | 211.3 |
| administration | T_{1/2} (h) | 5.3 | 5.7 |
| (10 mg/kg) | AUC₀₋ₗₐₛₜ (h*ng/mL) | 491.2 | 1437.3 |
| | F % | 32.1 | 42.9 |

Compound A is compound 506 in WO2016185160A1, and the chemical structure is as follows:

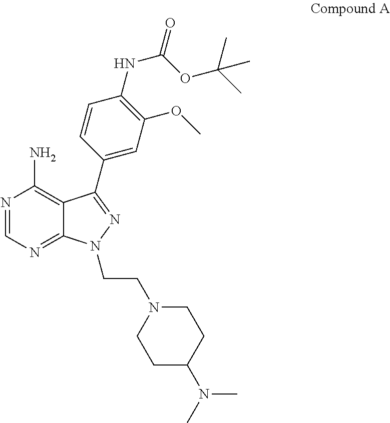 [SMILES: COc1cc(-c2nn(CCN3CCC(N(C)C)CC3)c3ncnc(N)c23)ccc1NC(=O)OC(C)(C)C]

As can be seen from the data in Table 6, in mouse pharmacokinetic experiments, compared to compound A, compound 59 of the present invention has a half-life Ti/₂ similar to compound A, indicating that compound A has similar metabolic stability to compound 59. However, compound 59 showed a great increase in Vdss, indicating that compound 59 is less distributed in plasma and more distributed in other tissues, with greater advantages for treating solid tumors. Thus, compound 59 has unexpectedly improved pharmacokinetic properties compared to compound A.

### Example 75. Stability Experiments

5 mg each of compound 59 and compound A was dissolved in an aqueous hydrochloric acid solution (5 mL) with a pH of 2. The mixture was stirred at room temperature. Samples were taken after 0.5 h for HPLC assay using the following assay conditions. The HPLC assay results are shown in Table 7.
Instrument: Agilent 1260 infinity 2
Column: Xtimate C18 4.6*150 mm,3 um
Temperature:25° C.
Wave length:210 nm/254 nm
phase A: H₂O (0.1% TFA)
phase B: MECN (0.1% TFA)
Gradient:

| Time (min) | Flow (mL/min) | B % | A % |
|---|---|---|---|
| 0 | 1 | 5 | 95 |
| 1 | 1 | 5 | 95 |
| 20 | 1 | 50 | 50 |
| 24 | 1 | 90 | 10 |
| 28 | 1 | 90 | 10 |
| 28.01 | 1 | 5 | 95 |
| 35 | 1 | 5 | 95 |

TABLE 7
Comparison of stability

| | | Purity after stirring in an aqueous |
|---|---|---|
| | Initial | hydrochloric acid solution |
| Compound | purity | with a pH of 2 for 0.5 h |
| Compound 59 | 96.35% | 96.16% |
| Compound A | 96.18% | Complete degradation |

As can be seen from Table 7, compound 59 of the present invention is more stable under acidic conditions than compound A.

Although specific embodiments of the present invention have been described above, it will be appreciated by those skilled in the art that these embodiments are merely illustrative and that many changes or modifications can be made to these embodiments without departing from the principles and spirit of the present invention. The protection scope of the present invention is therefore defined by the appended claims.

1. A compound of general formula (1) or an isomer, a crystalline form, a pharmaceutically acceptable salt, a hydrate or a solvate thereof: 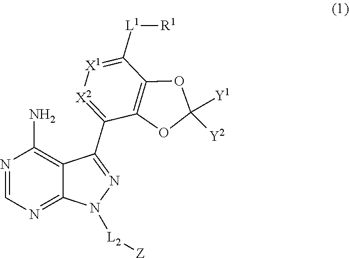
wherein in general formula (1):
X¹ is CR^{a1} or N;
X² is CR^{a2} or N;
Y¹ is H or F;
Y² is H or F;
L¹ is —C(O)—, —NR²C(O)—, —NR²C(O)O—, —OC(O)NR²—, —C(O)NR²—, —NR³C(O)NR²—, —NR²S(O)₂—, —S(O)ₚ—, —S(O)₂NR²— or —NR³S(O)₂NR²—;
R¹ is —H, —OH, —CN, (C1-C6) alkyl, (C1-C6) haloalkyl, (C2-C6) alkenyl, (C2-C6) alkynyl, (C3-C6) cycloalkyl, phenyl, (5-9 membered) heteroaryl or (4-6 membered) heterocycloalkyl, wherein the (C1-C6) alkyl, (C1-C6) haloalkyl, (C2-C6) alkenyl, (C2-C6) alkynyl, (C3-C6) cycloalkyl, phenyl, (5-9 membered) heteroaryl or (4-6 membered) heterocycloalkyl can be each independently and optionally substituted with 1, 2, 3 or 4 of the following groups: —H, halogen, —OH, —OR⁵, —CN, (C1-C6) alkyl and (C1-C6) haloalkyl;
L² is (C1-C6) alkylene, wherein the (C1-C6) alkylene can be independently and optionally substituted with 1, 2, 3 or 4 of the following groups: —H, halogen, —OH, —OR⁵, —CN, (C1-C6) alkyl, (C1-C6) haloalkyl and 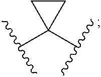 [SMILES: *C1(*)CC1]
Z is —OR⁷, —NR⁷R⁸, —NR⁶(CH₂)ₘOR⁷, —NR⁶(CH₂)ₘNR⁷R⁸, —O(CH₂)ₘOR⁶, —O(CH₂)ₘNR⁷R⁸ or (4-12 membered) heterocycloalkyl, wherein the (4-12 membered) heterocycloalkyl can be each independently and optionally substituted with 1, 2, 3 or 4 of the following groups: —H, halogen, —OH, —OR⁴, —CN, (C1-C6) alkyl, (C1-C6) haloalkyl, (C3-C6) cycloalkyl, —NR⁷R⁸, —NR⁶(CH₂)ₘOR⁷, —O(CH₂)ₘOR⁶, —O(CH₂)ₘNR⁷R⁸, —NR⁶(CH₂)ₘNR⁷R⁸, —(CH₂)ₘOR⁷, —(CH₂)ₘNR⁷R⁸ and R⁹;
R^{a1} and R^{a2} are each independently —H, —OR⁵, —CN or halogen;
R² and R³ are each independently —H, (C1-C6) alkyl, (C1-C6) haloalkyl, (C2-C6) alkenyl, (C2-C6) alkynyl or (C3-C6) cycloalkyl;
R⁴ is —H, (C1-C6) alkyl, (C1-C6) haloalkyl, (C2-C6) alkenyl, (C2-C6) alkynyl or (C3-C6) cycloalkyl;
R⁵ is —H, (C1-C6) alkyl, (C1-C6) haloalkyl or (C3-C6) cycloalkyl;
R⁶, R⁷ and R⁸ are each independently —H, (C1-C6) alkyl, (C1-C6) haloalkyl, (C2-C6) alkenyl, (C2-C6) alkynyl, (C3-C6) cycloalkyl, (4-12 membered) heterocycloalkyl, phenyl or (5-6 membered) heteroaryl, wherein the (C1-C6) alkyl, (C1-C6) haloalkyl, (C2-C6) alkenyl, (C2-C6) alkynyl, (C3-C6) cycloalkyl, (4-12 membered) heterocycloalkyl, phenyl or (5-6 membered) heteroaryl can be each independently and optionally substituted with 1, 2, 3 or 4 of the following groups: —H, halogen, —OH, —OR¹⁰, —CN, (C1-C6) alkyl and (C1-C6) haloalkyl; or R⁷ and R⁸, together with the N atom to which they are linked, can form (4-12 membered) heterocycloalkyl, wherein the (4-12 membered) heterocycloalkyl can be optionally substituted with 1, 2, 3 or 4 of the following groups: —H, halogen, —OH, —OR¹⁰, —CN, (C1-C6) alkyl, (C1-C6) haloalkyl, (C1-C6) cycloalkyl, —NR¹⁰R¹¹, —NR¹⁰(CH₂)ₘOR¹¹, —O(CH₂)ₘOR¹⁰, —O(CH₂)ₘNR¹⁰R¹¹, —NR¹⁰(CH₂)ₘNR¹¹R¹², (CH₂)ₘOR⁷, —(CH₂)ₘNR⁷R⁸ and R⁹;
R⁹ is —H or (4-9 membered) heterocycloalkyl, wherein the (4-9 membered) heterocycloalkyl can be independently and optionally substituted with 1, 2, 3 or 4 of the following groups: —H, —OH, —OR¹⁰, —CN, (C1-C6) alkyl, (C1-C6) haloalkyl or (C3-C6) cycloalkyl;
R¹⁰, R¹¹ and R¹² are each independently —H, (C1-C6) alkyl, (C1-C6) haloalkyl, (C3-C6) cycloalkyl or (4-6 membered) heterocycloalkyl; and
p is an integer of 0, 1 or 2, and m is an integer of 1, 2 or 3.

2. The compound or the isomer, the crystalline form, the pharmaceutically acceptable salt, the hydrate or the solvate thereof according to claim 1, wherein in general formula (1), L¹ is —C(O)—, —NHC(O)O—, —OC(O)NH—, —C(O)NH—, —NHC(O)NH—, —NHS(O)₂—, —S(O)₂NH—, —NHS(O)₂NH—, —N(CH₃)C(O)—, —N(CH₃)C(O)O—, —OC(O)N(CH₃)—, —C(O)N(CH₃)—, —N(CH₃)C(O)NH—, —NHC(O)N(CH₃)—, —N(CH₃)C(O)N(CH₃)—, —N(CH₃)S(O)₂—, —S(O)₂—, —S(O)—, —S—, —S(O)₂N(CH₃)—, —N(CH₃)S(O)₂NH—, —NHS(O)₂N(CH₃)— or —N(CH₃)S(O)₂N(CH₃)—; L¹ is preferably —NHC(O)—, —NHC(O)O—, —NHC(O)NH—, —NHC(O)O—, —OC(O)NH— or —C(O)NH—; L¹ is more preferably —NHC(O)—, —NHC(O)O— or —NHC(O)NH—; L¹ is more preferably —NHC(O)O—.

3. The compound or the isomer, the crystalline form, the pharmaceutically acceptable salt, the hydrate or the solvate thereof according to claim 1, wherein in general formula (1), R¹ is —H, —OH, —CN, (C1-C5) alkyl, (C1-C5) haloalkyl, (C2-C5) alkenyl, (C2-C5) alkynyl, (C3-C6) cycloalkyl, phenyl, (5-9 membered) heteroaryl or (4-6 membered) heterocycloalkyl, wherein the (C1-C5) alkyl, (C1-C5) haloalkyl, (C2-C5) alkenyl, (C2-C5) alkynyl, (C3-C6) cycloalkyl, phenyl, (5-9-membered) heteroaryl or (4-6 membered) heterocycloalkyl can be each independently and optionally substituted with 1, 2, 3 or 4 of the following groups: —H, —F, —Cl, —Br, —OH, —OCH₃, —CN, (C1-C3) alkyl and (C1-C3) haloalkyl.

4. The compound or the isomer, the crystalline form, the pharmaceutically acceptable salt, the hydrate or the solvate thereof according to claim 3, wherein in general formula (1), R¹ is:
—H, —OH, —CN, 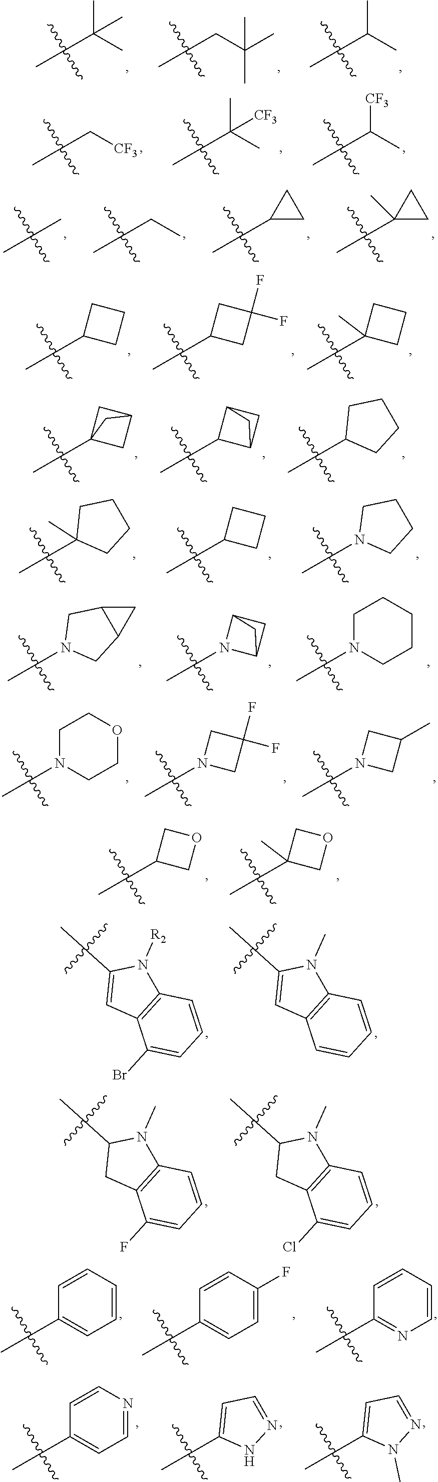 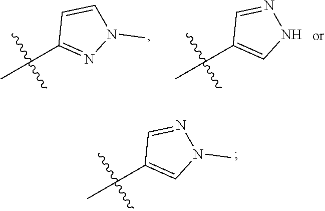
preferably, 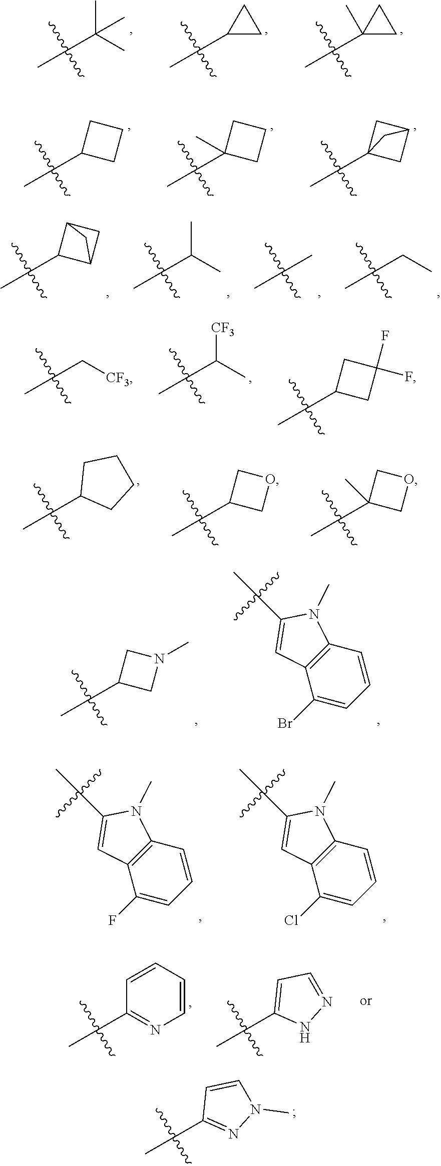
more preferably 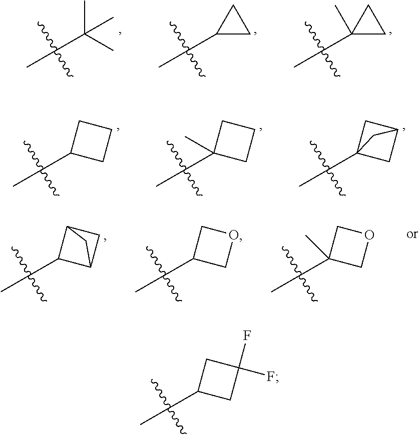
more preferably 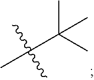 [SMILES: *C(C)(C)C]
and more preferably

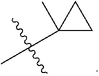 [SMILES: *C1(C)CC1]

5. The compound or the isomer, the crystalline form, the pharmaceutically acceptable salt, the hydrate or the solvate thereof according to claim 1, wherein in general formula (1), L² is (C1-C4) alkylene, wherein the (C1-C4) alkylene can be each independently and optionally substituted with 1, 2, 3 or 4 of the following groups: —H, —F, —OH, —OCH₃, —CN, (C1-C3) alkyl, (C1-C3) haloalkyl and

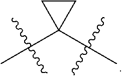

6. The compound or the isomer, the crystalline form, the pharmaceutically acceptable salt, the hydrate or the solvate thereof according to claim 5, wherein in general formula (1), L² is: 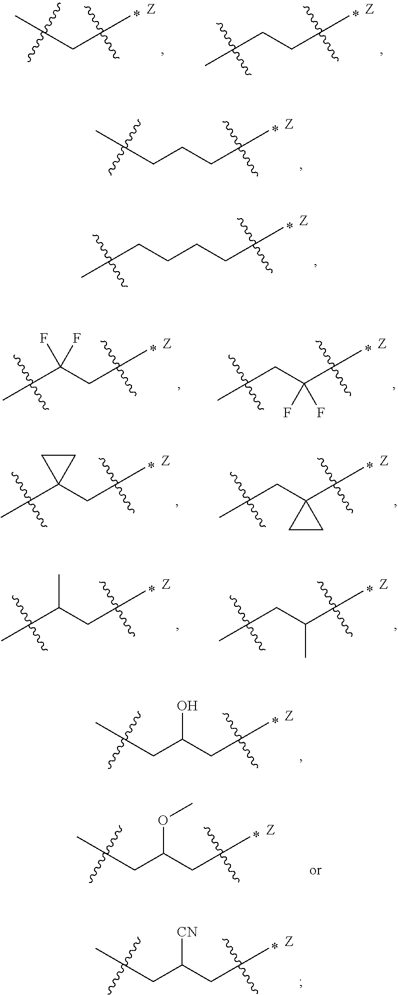
preferably 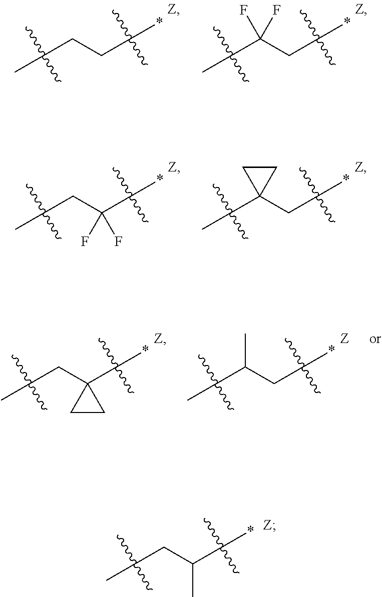
and more preferably

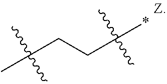

7. The compound or the isomer, the crystalline form, the pharmaceutically acceptable salt, the hydrate or the solvate thereof according to claim 1, wherein in general formula (1), Z is —NR⁷R⁸, —NR⁶(CH₂)₂OR⁷, —NR⁶(CH₂)₂NR⁷R⁸, —O(CH₂)₂OR⁶, —O(CH₂)₂NR⁷R⁸ or (4-11 membered) heterocycloalkyl, wherein the (4-11 membered) heterocycloalkyl can be independently and optionally substituted with 1, 2, 3 or 4 of the following groups: —H, —F, —OH, —OCH₃, —CN, (C1-C3) alkyl, (C1-C3) haloalkyl, (C3-C6) cycloalkyl, —NR⁷R⁸, —NR⁶(CH₂)₂OR⁷, —O(CH₂)₂OR⁶, —O(CH₂)₂NR⁷R⁸, —NR⁶(CH₂)₂NR⁷R⁸, —(CH₂)₂OR⁷, —(CH₂)₂NR⁷R⁸, —CH₂OR⁷, —CH₂NR⁷R⁸ and R⁹.

8. The compound or the isomer, the crystalline form, the pharmaceutically acceptable salt, the hydrate or the solvate thereof according to claim 7, wherein in general formula (1), R⁶, R⁷ and R⁸ are each independently —H, (C1-C3) alkyl, (C1-C3) haloalkyl, (C3-C6) cycloalkyl, (4-9 membered) heterocycloalkyl, phenyl or (5-6 membered) heteroaryl, wherein the (C1-C3) alkyl, (C1-C3) haloalkyl, (C3-C6) cycloalkyl, (4-9 membered) heterocycloalkyl, phenyl or (5-6 membered) heteroaryl can be each independently and optionally substituted with 1, 2, 3 or 4 of the following groups: —H, —F, —OH, —OCH₃, —CN, (C1-C3) alkyl and (C1-C3) haloalkyl; or R⁷ and R⁸, together with the N atom to which they are linked, can form (4-11 membered) heterocycloalkyl, wherein the (4-11 membered) heterocycloalkyl can be optionally substituted with 1, 2, 3 or 4 of the following groups: —H, —F, —OH, —OCH₃, —CN, (C1-C3) alkyl, (C1-C3) haloalkyl, (C1-C3) cycloalkyl, —NR¹⁰R¹¹, —NR¹⁰(CH₂)₂OR¹¹, —O(CH₂)₂OR¹⁰, —O(CH₂)₂NR¹⁰R¹¹, —NR¹⁰(CH₂)₂NR¹¹R¹², —(CH₂)₂OR⁷, —(CH₂)₂NR⁷R⁸, —CH₂OR⁷, —CH₂NR⁷R⁸ and R⁹.

9. The compound or the isomer, the crystalline form, the pharmaceutically acceptable salt, the hydrate or the solvate thereof according to claim 7, wherein in general formula (1), Z is: 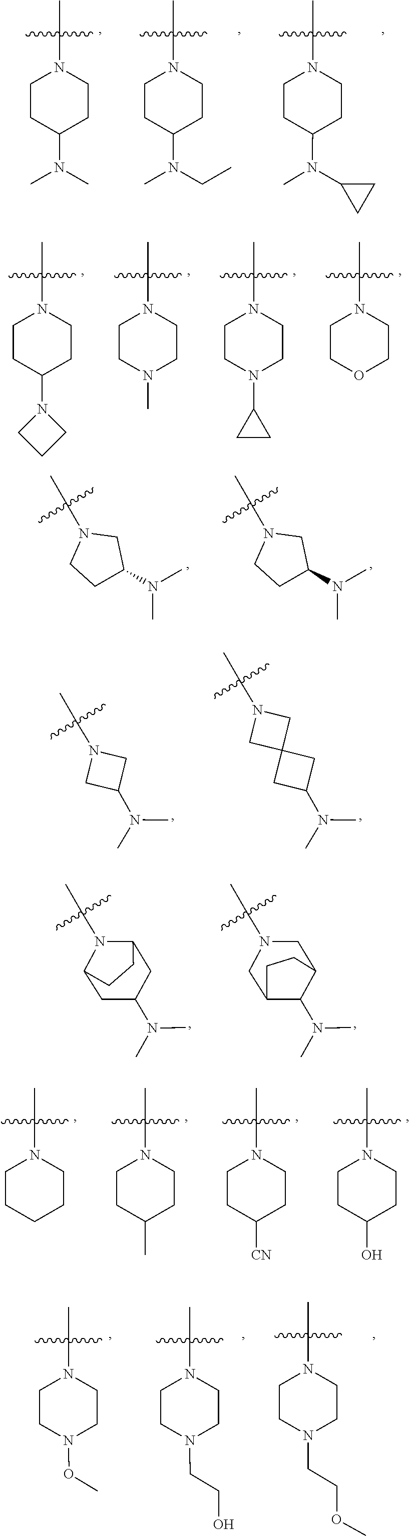 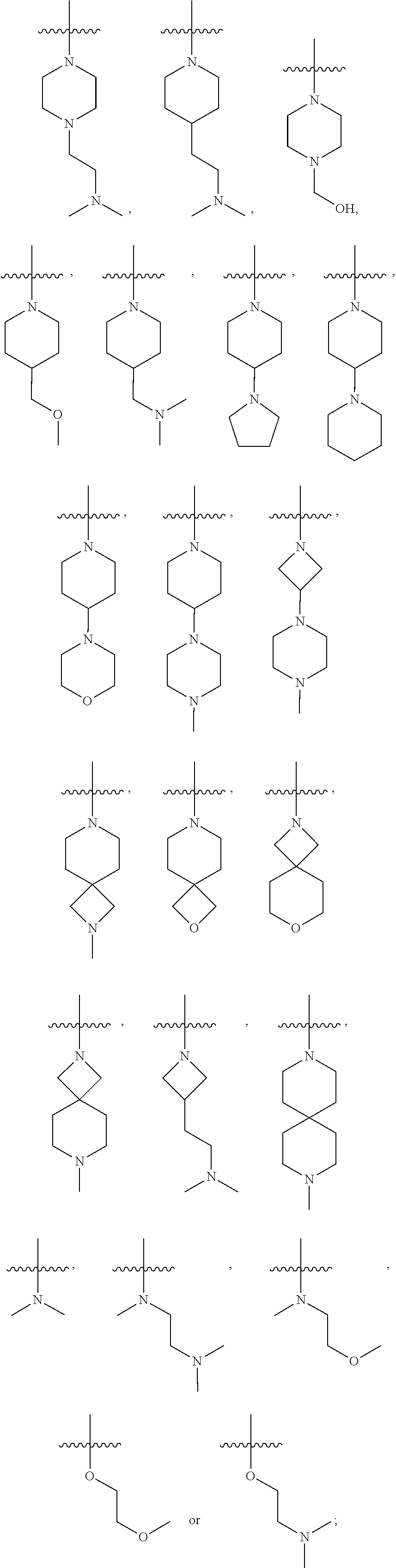
preferably 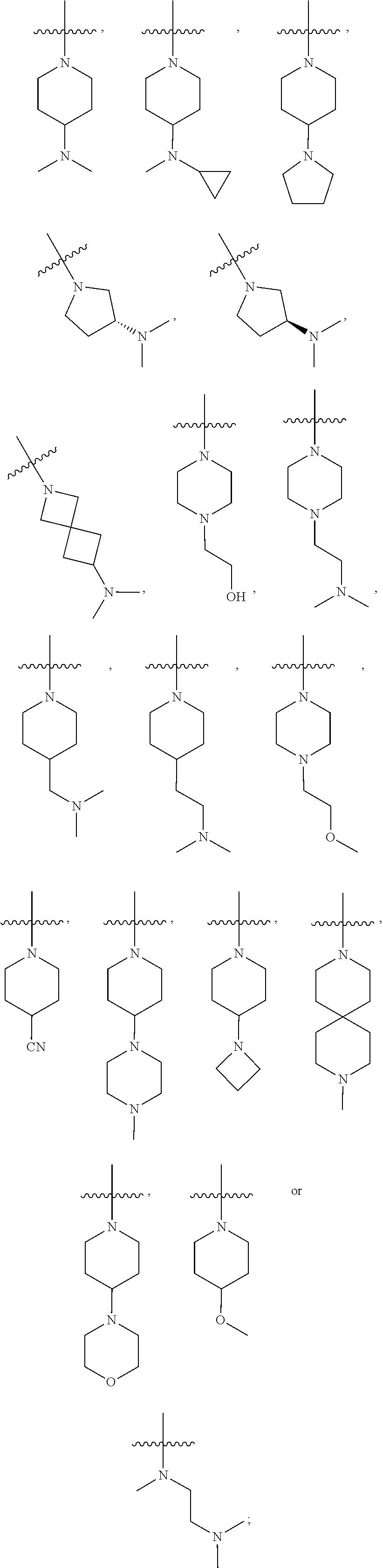
more preferably

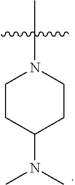

10. The compound or the isomer, the crystalline form, the pharmaceutically acceptable salt, the hydrate or the solvate thereof according to claim 1, wherein in general formula (1), R^{a1} and R^{a2} are each independently —H or —OCH₃, —CN or —F, preferably —H or —F.

11. The compound or the isomer, the crystalline form, the pharmaceutically acceptable salt, the hydrate or the solvate thereof according to claim 1, wherein in general formula (1), Y¹ and Y² are each independently —H or —F, preferably —H.

12. The compound or the isomer, the crystalline form, the pharmaceutically acceptable salt, the hydrate or the solvate thereof according to claim 1, wherein in general formula (1), the compound has one of the following structures:

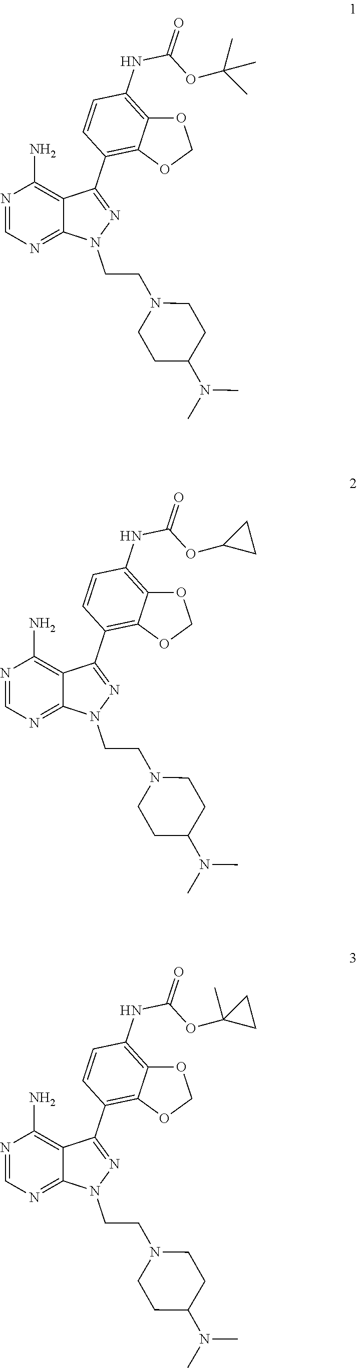

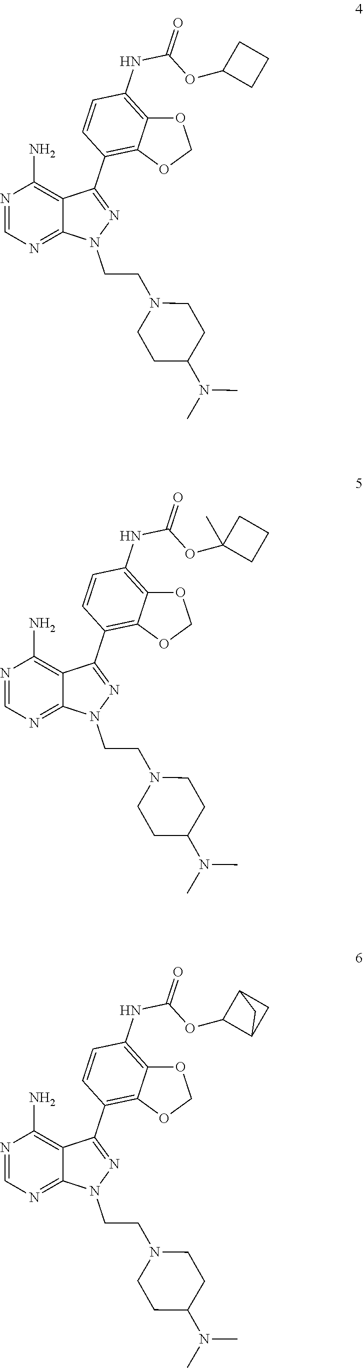

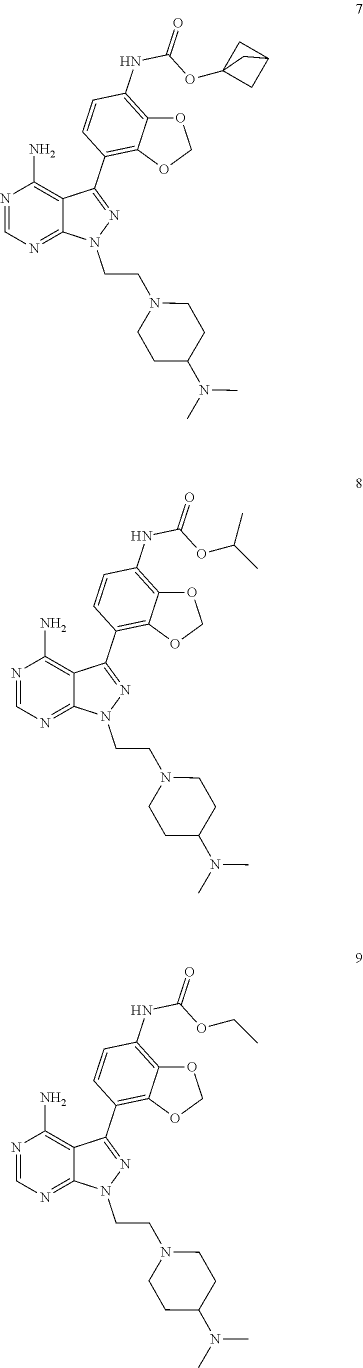

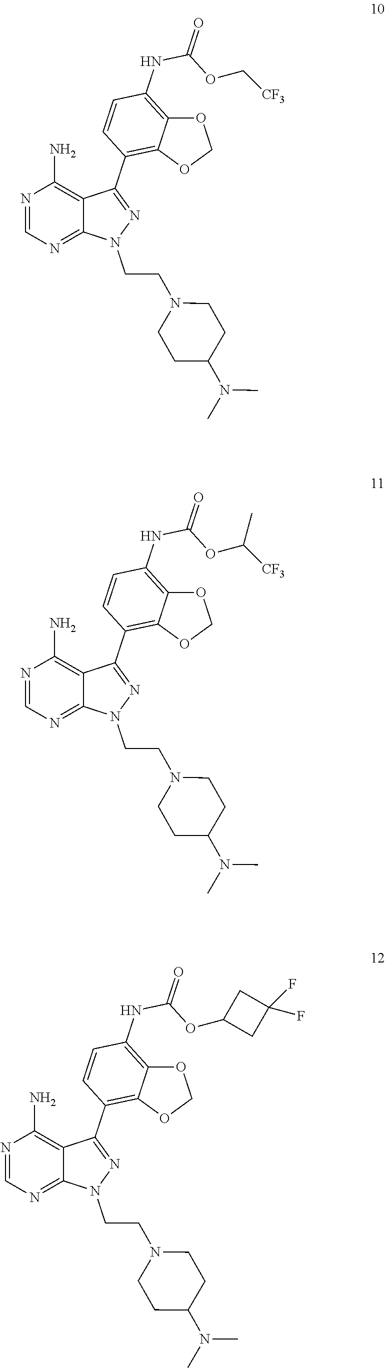

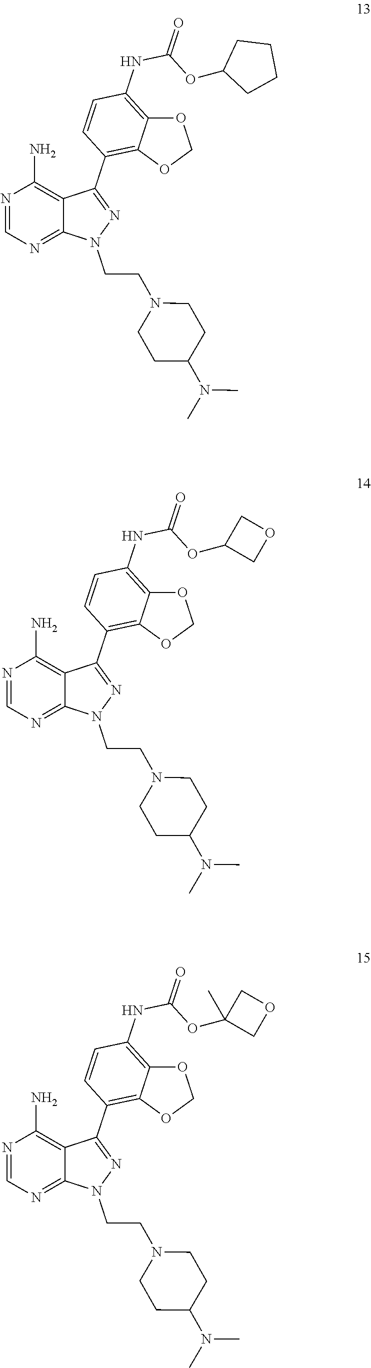

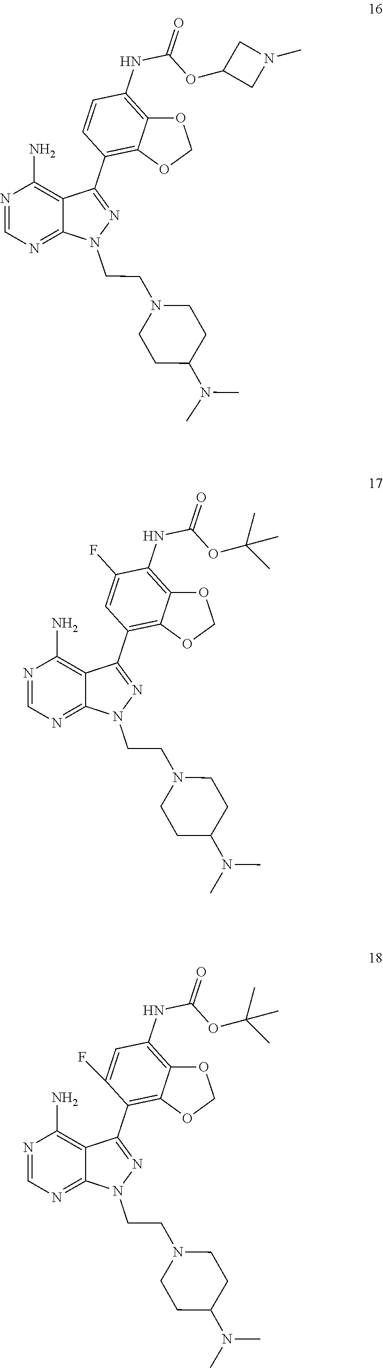

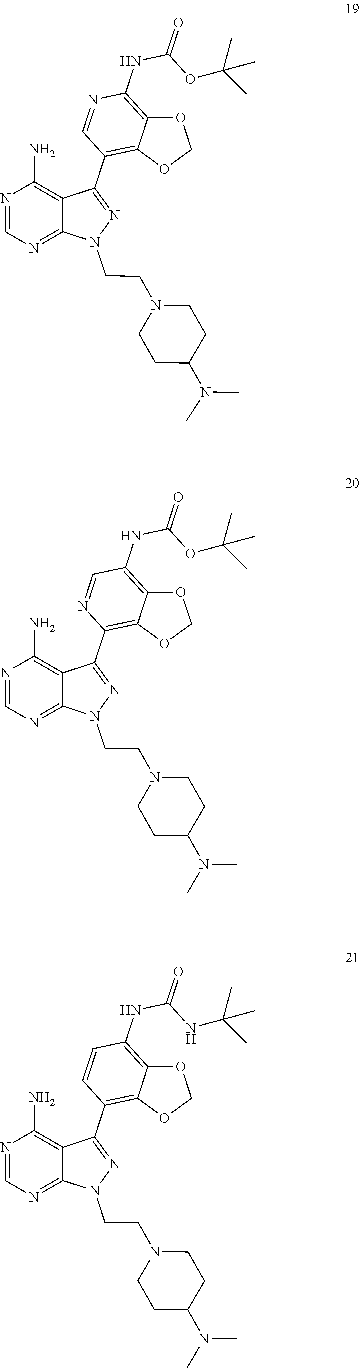

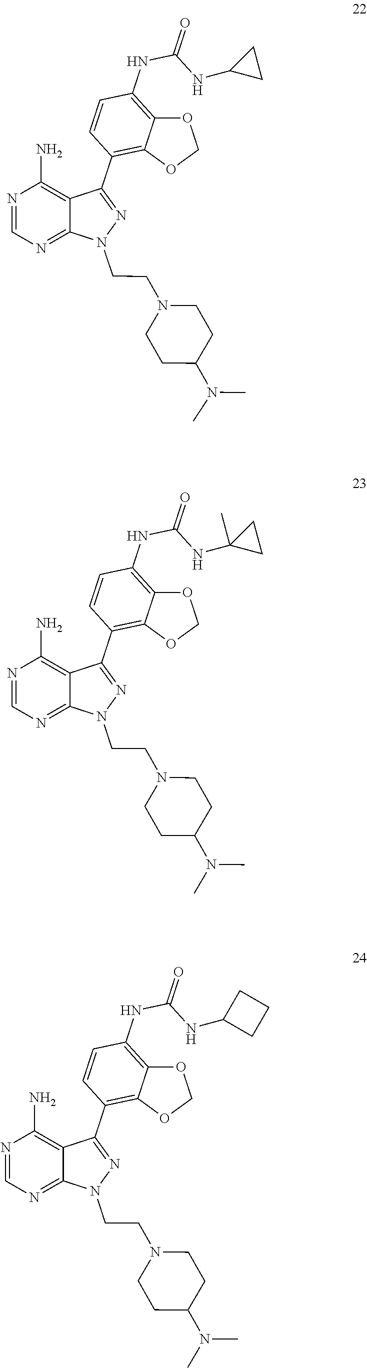

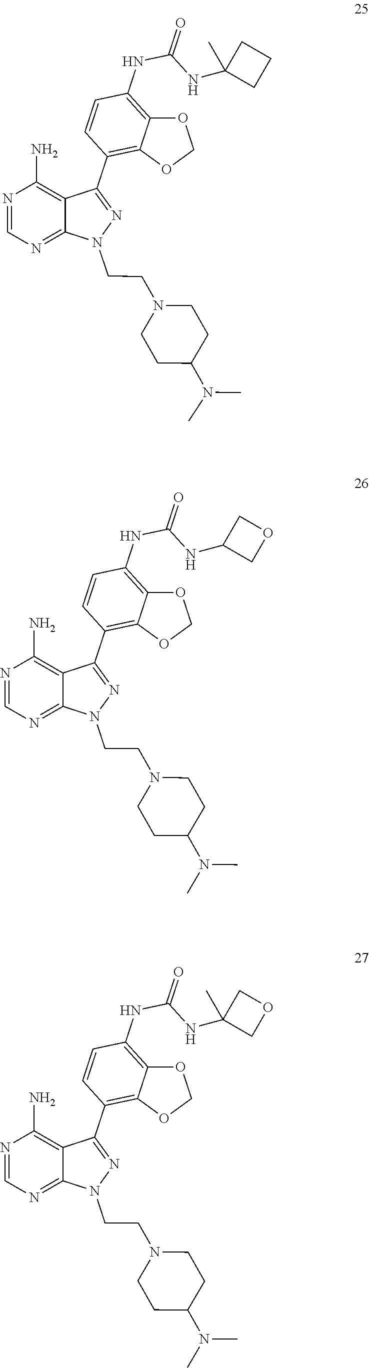

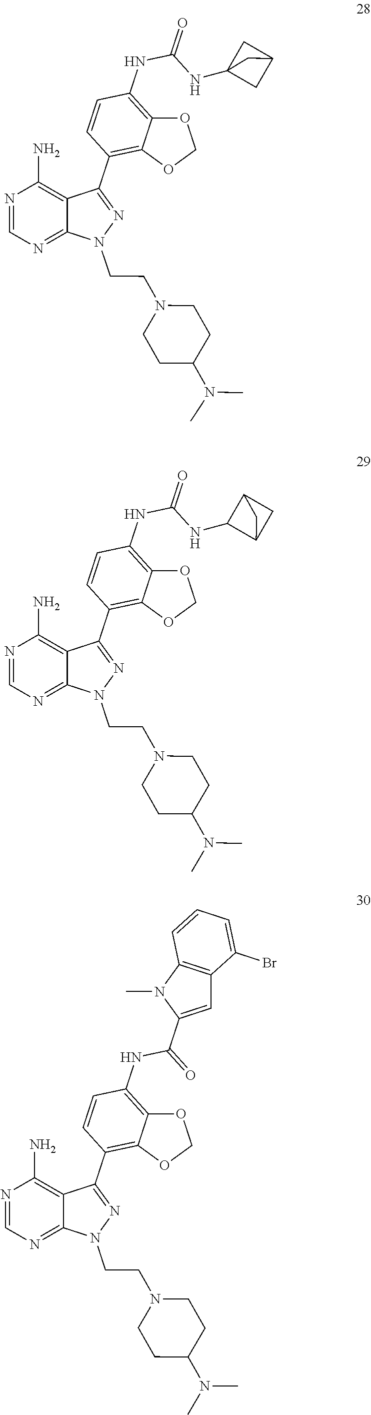

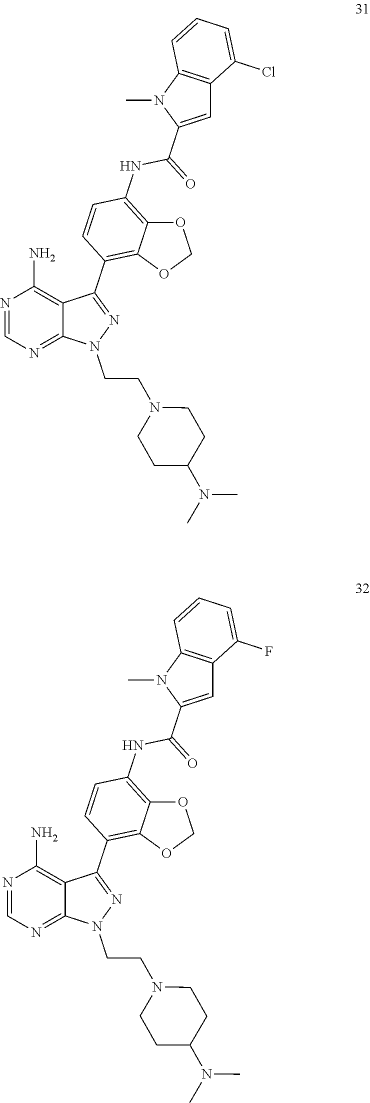

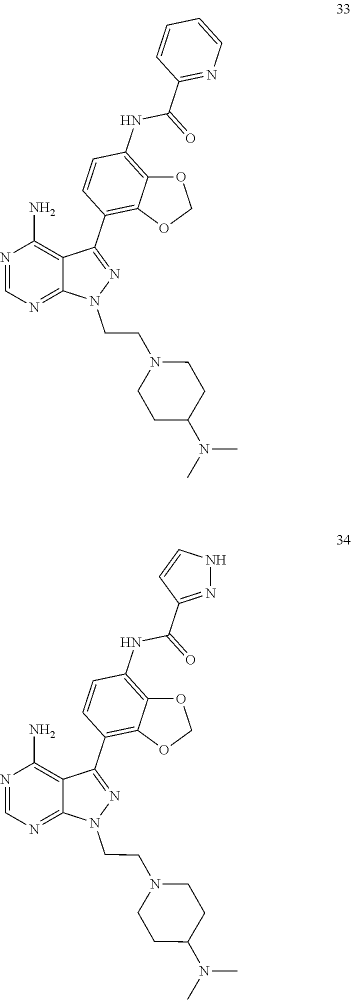

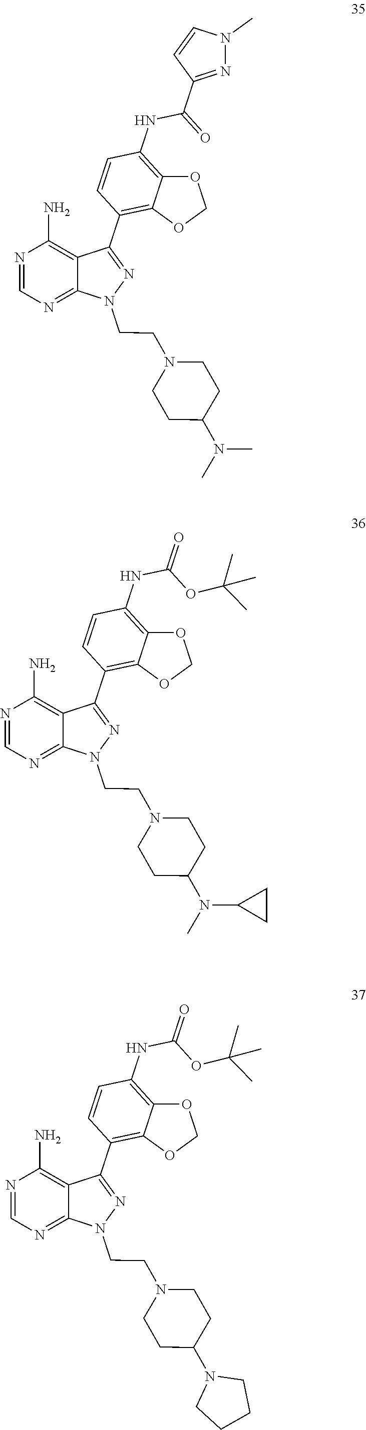

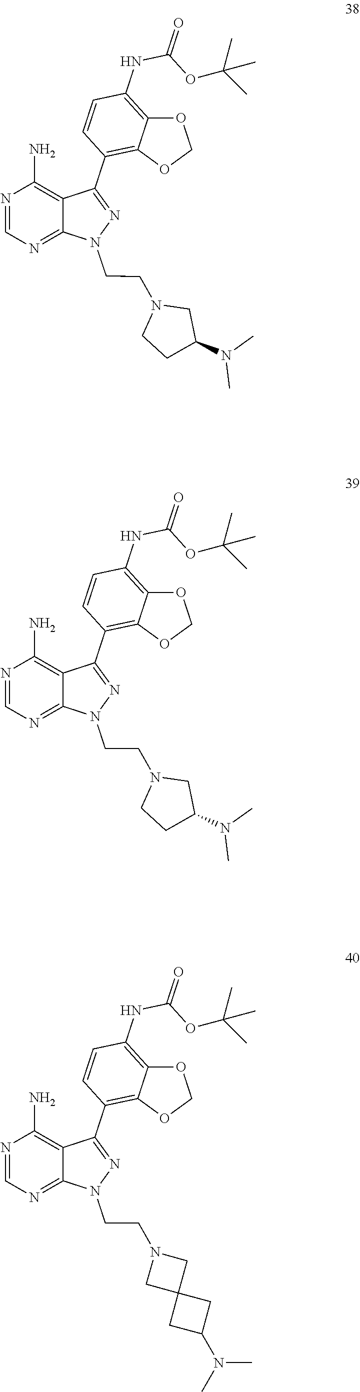

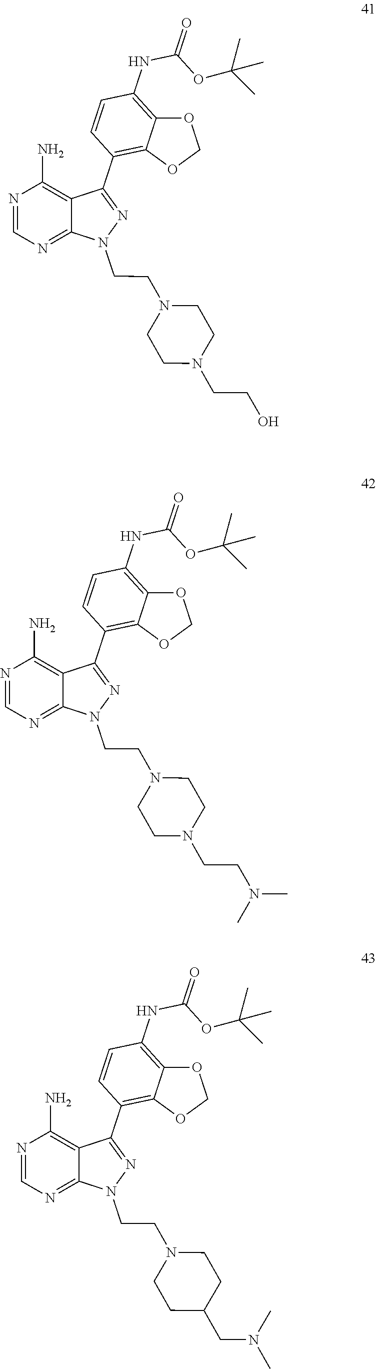

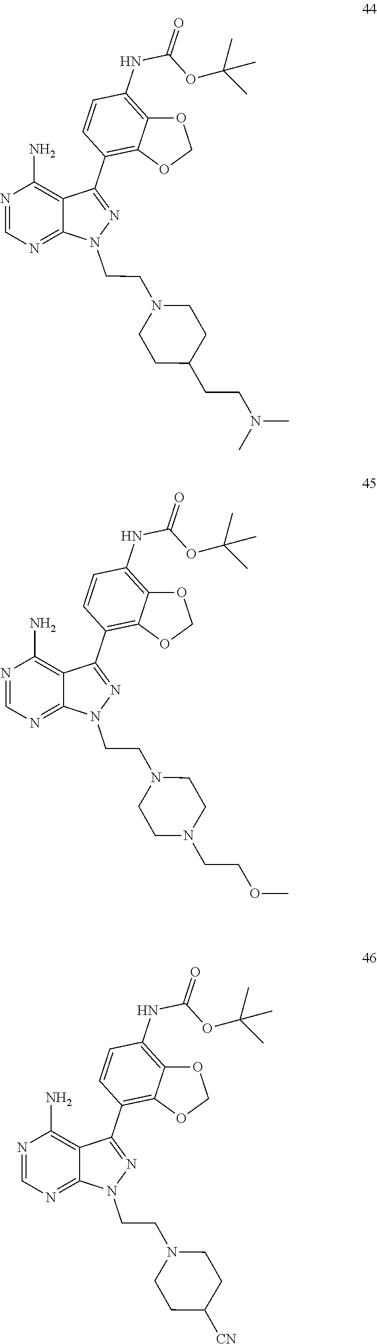

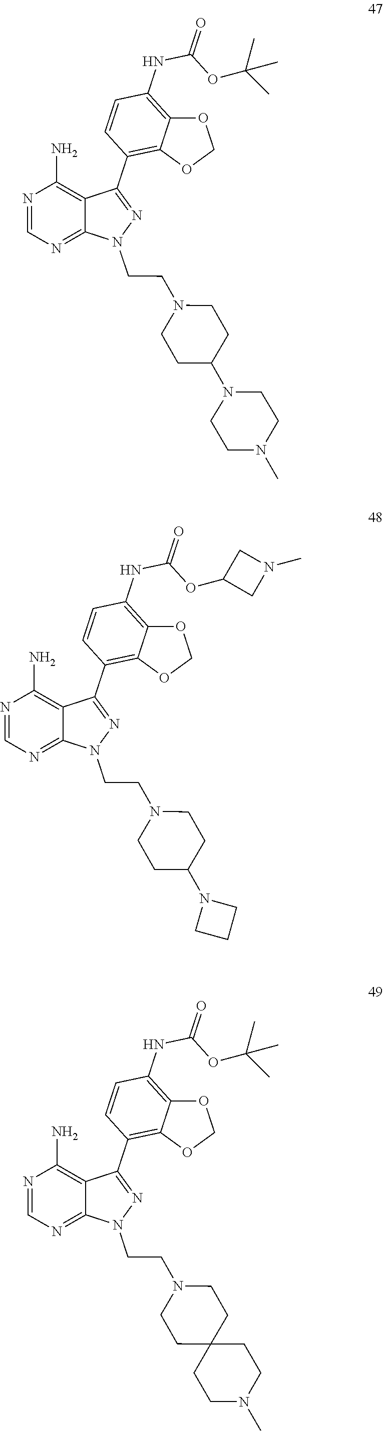

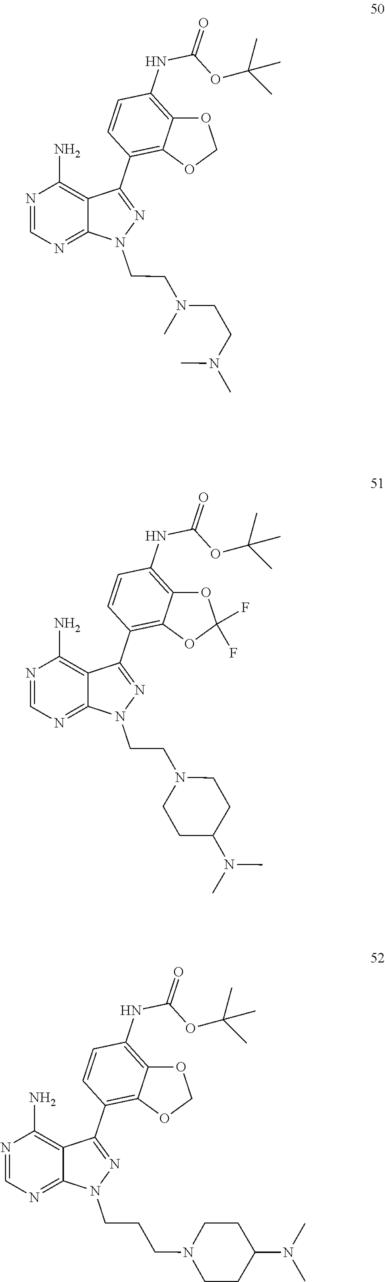

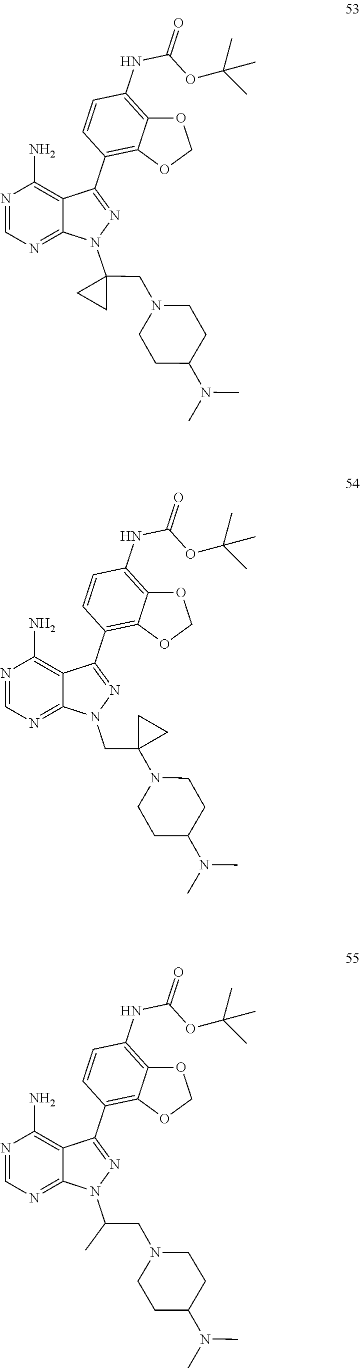

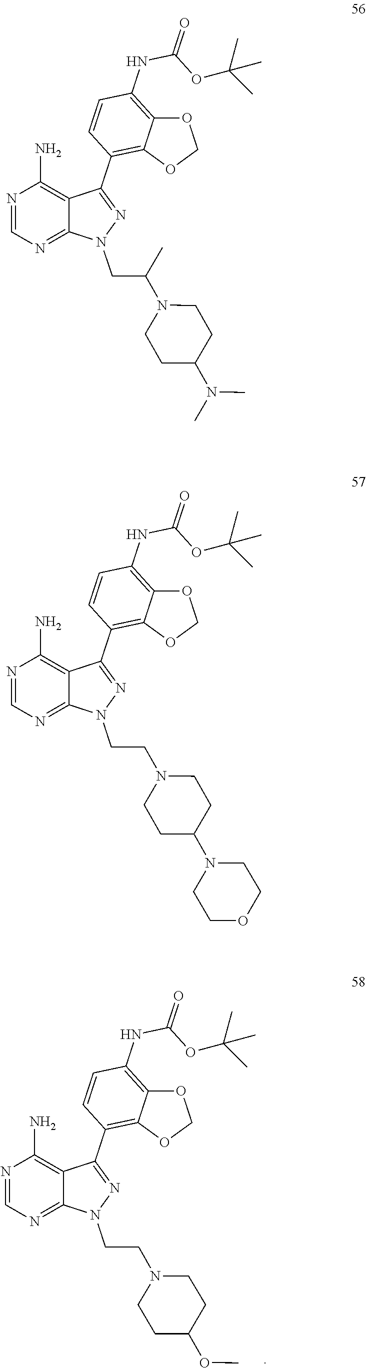

13. A compound of general formula (2) or an isomer, a crystalline form, a pharmaceutically acceptable salt, a hydrate or a solvate thereof: 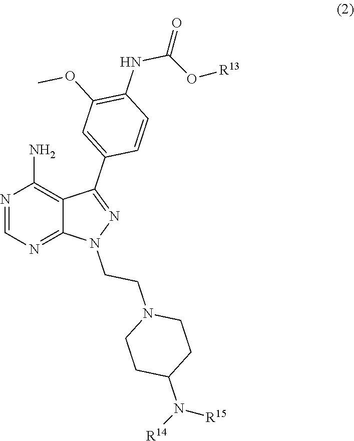
wherein in general formula (2):
R¹³ is (C1-C6) alkyl, (C3-C6) cycloalkyl or (4-6 membered) heterocycloalkyl, wherein the (C1-C6) alkyl, (C3-C6) cycloalkyl or (4-6 membered) heterocycloalkyl can be each independently and optionally substituted with 1, 2, 3 or 4 of the following groups: —H, halogen, —OH, —OR¹⁶, —CN, (C1-C6) alkyl and (C1-C6) haloalkyl;
R¹⁴ and R¹⁵ are each independently —H, (C1-C6) alkyl, (C1-C6) haloalkyl or (C3-C6) cycloalkyl, wherein the (C1-C6) alkyl, (C1-C6) haloalkyl or (C3-C6) cycloalkyl can be each independently and optionally substituted with 1, 2, 3 or 4 of the following groups: —H, halogen, —OH, —OR¹⁶, —CN, (C1-C6) alkyl and (C1-C6) haloalkyl;
when R¹³ is (C1-C6) alkyl, one of R¹⁴ and R¹⁵ must be (C3-C6) cycloalkyl;
R¹⁶ is —H, (C1-C6) alkyl, (C1-C6) haloalkyl or (C3-C6) cycloalkyl.

14. The compound or the isomer, the crystalline form, the pharmaceutically acceptable salt, the hydrate or the solvate thereof according to claim 13, wherein in general formula (2), R¹³ is (C1-C6) alkyl, (C3-C6) cycloalkyl or (4-6 membered) heterocycloalkyl, wherein the (C1-C6) alkyl, (C3-C6) cycloalkyl or (4-6 membered) heterocycloalkyl can be each independently and optionally substituted with 1, 2, 3 or 4 of the following groups: H, —F, —Cl, —Br, —OH, —OCH₃, —CN, (C1-C3) alkyl and (C1-C3) haloalkyl.

15. The compound or the isomer, the crystalline form, the pharmaceutically acceptable salt, the hydrate or the solvate thereof according to claim 14, wherein in general formula (2), R¹³ is: 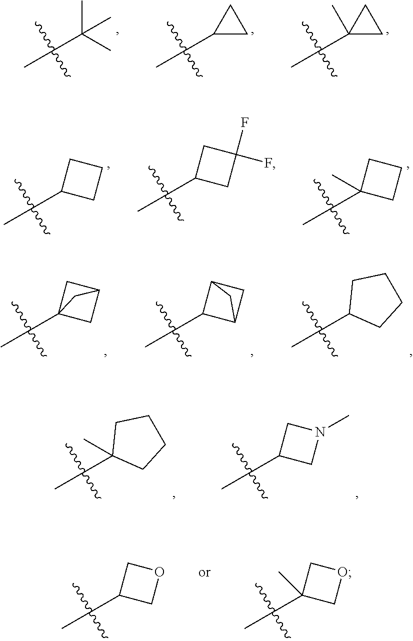
preferably 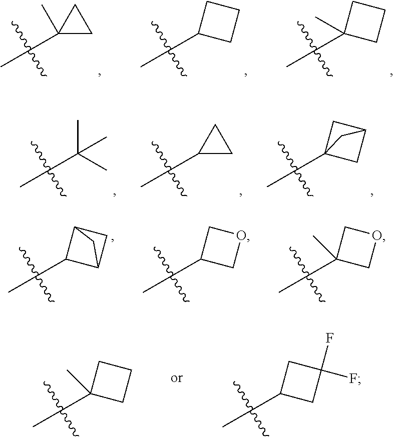
more preferably 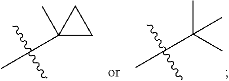
more preferably 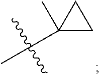 [SMILES: *C1(C)CC1]
and more preferably

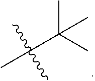 [SMILES: *C(C)(C)C]

16. The compound or the isomer, the crystalline form, the pharmaceutically acceptable salt, the hydrate or the solvate thereof according to claim 1, wherein in general formula (2), R¹⁴ and R¹⁵ are each independently —H, (C1-C3) alkyl, (C1-C3) haloalkyl or (C3-C6) cycloalkyl, wherein the (C1-C3) alkyl, (C1-C3) haloalkyl or (C3-C6) cycloalkyl can be each independently and optionally substituted with 1, 2, 3 or 4 of the following groups: —H, —F, —OH, —OCH₃, —CN, (C1-C3) alkyl and (C1-C3) haloalkyl; when R¹³ is (C1-C6) alkyl, one of R¹⁴ and R¹⁵ must be (C3-C6) cycloalkyl.

17. The compound or the isomer, the crystalline form, the pharmaceutically acceptable salt, the hydrate or the solvate thereof according to claim 16, wherein in general formula (2), structural unit 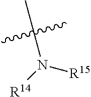
is selected from: 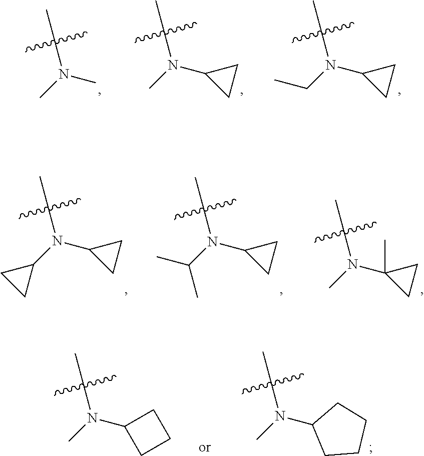
preferably 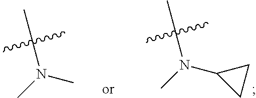
more preferably 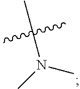
and more preferably

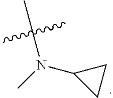

18. The compound or the isomer, the crystalline form, the pharmaceutically acceptable salt, the hydrate or the solvate thereof according to claim 13, wherein in general formula (2), the compound has one of the following structures:

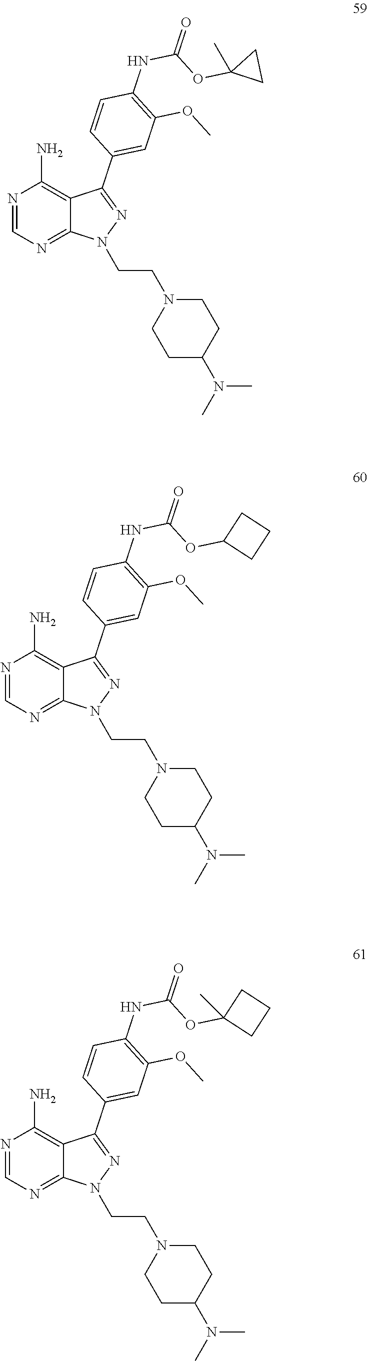

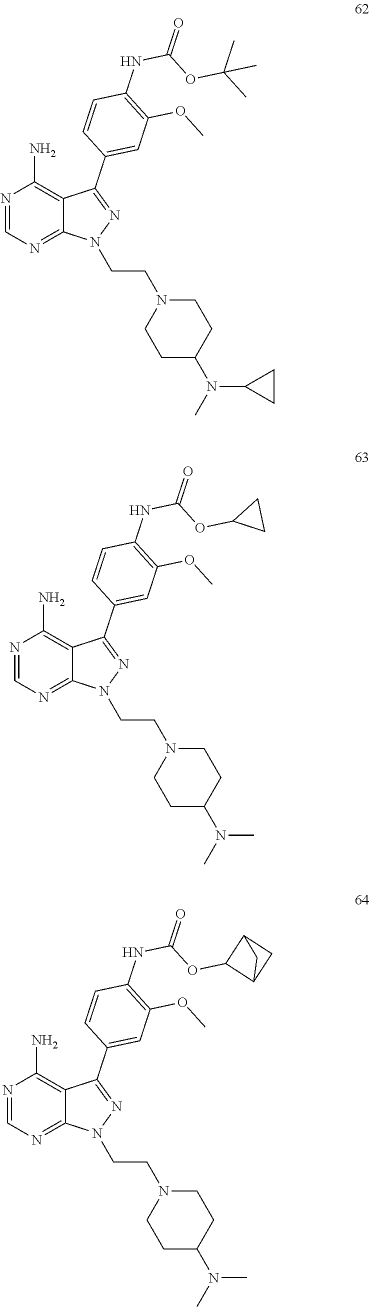

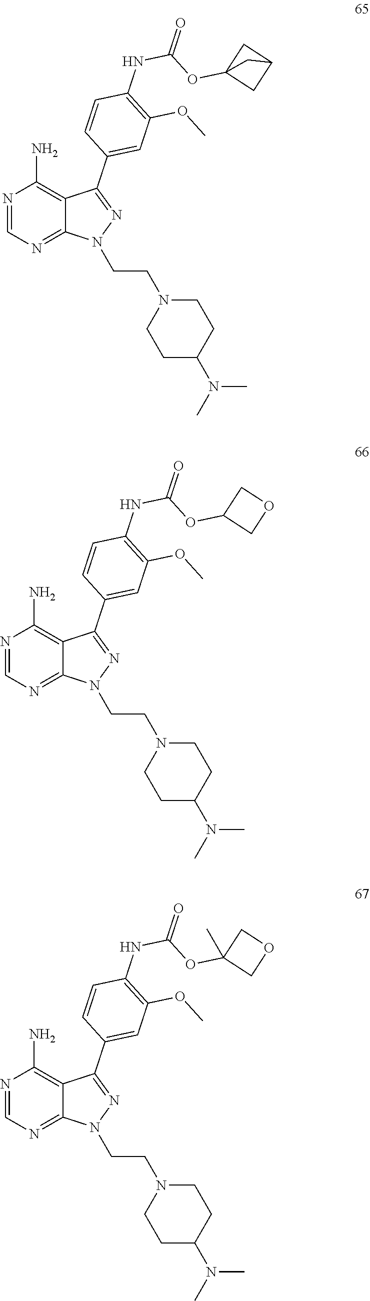

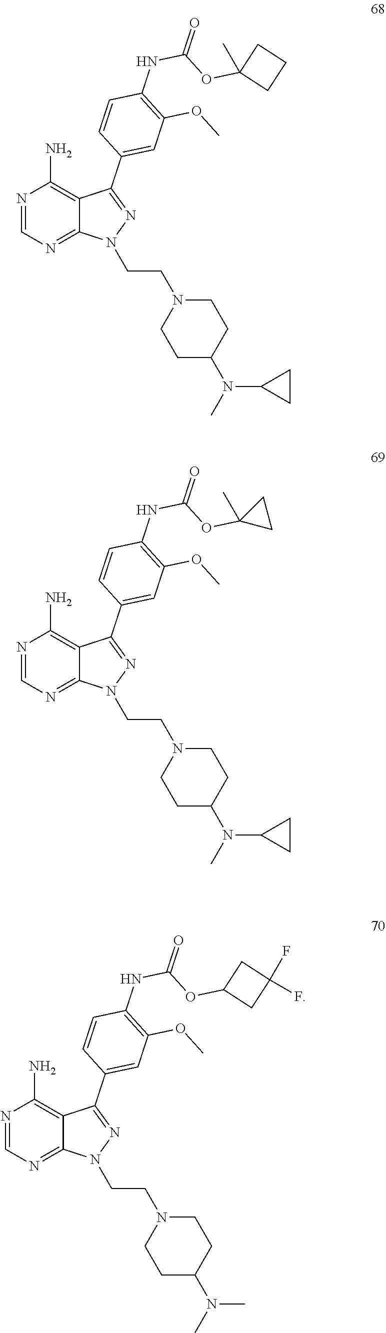

19. A pharmaceutical composition, comprising a pharmaceutically acceptable excipient or carrier, and the compound or the isomer, the crystalline form, the pharmaceutically acceptable salt, the hydrate or the solvate thereof according to claim 1 as an active ingredient.

20. (canceled)

21. (canceled)
